(19) United States
(12) Patent Application Publication
(10) Pub. No.: US 2024/0294626 A1
(43) Pub. Date: Sep. 5, 2024

(54) BISPECIFIC ANTIBODIES FOR USE IN TREATMENT OF HIDRADENITIS SUPPURATIVA

(71) Applicant: NOVARTIS AG, Basel (CH)

(72) Inventors: Sabine BECK, Sasbach (DE); Michael KIFFE, Efringen-Kirchen (DE); Jiri KOVARIK, Zürich (CH); Christian LOESCHE, Hegenheim (FR); Margarida RODRIGUES, Ericeira (PT); Richard STEIN, Weil am Rhein (DE); Yu TANG, Acton, MA (US); Frank WALDRON-LYNCH, Athboy, Co. Meath (IE)

(21) Appl. No.: 18/572,747

(22) PCT Filed: Jun. 20, 2022
(86) PCT No.: PCT/IB2022/055690

§ 371 Date: Dec. 20, 2023

Related U.S. Application Data
(60) Provisional application No. 63/213,686, filed on Jun. 22, 2021.
(60) Provisional application No. 63/223,479, filed on Jul. 19, 2021.

(51) Int. Cl. C07K 16/24; A61K 39/00

(52) U.S. Cl. CPC C07K 16/244; C07K 16/245; A61K 2039/505; A61K 2039/545; C07K 2317/31; C07K 2317/76; C07K 2317/92

(57) ABSTRACT

The invention relates to bivalent bispecific monoclonal antibodies (bbmAb) or variants thereof for use in the treatment or for use in alleviating the symptoms of hidradenitis suppurativa in a subject.

### TECHNICAL FIELD

The invention relates to bivalent bispecific monoclonal antibodies (bbmAb) or variants thereof, for the use in treatment of patients suffering from Hidradenitis Suppurativa. The disclosure also relates to methods and treatment regimens for treating Hidradenitis Suppurativa by employing a bispecific antibody that targets both IL-1β and IL-18 simultaneously.

### BACKGROUND OF THE DISCLOSURE

Hidradenitis suppurativa (HS), also called “acne inversa” or “maladie de Verneuilh”, is a chronic, recurrent, and debilitating inflammatory skin condition that typically presents with deep, inflammatory, painful lesions in apocrine gland-bearing parts of the body. The most common areas affected are the axillae, the groin, and the anogenital region (Jemec 2012; Fimmel and Zouboulis 2010).

HS is currently considered to be an inflammatory disease of the pilosebaceous follicle with an underlying immune system imbalance that occurs in genetically predisposed individuals (Kelly et al 2014). While it is considered a disease primarily triggered by follicular occlusion, HS is an inflammatory skin disease characterized by large numbers of neutrophils and macrophages in inflammatory lesions (Lima et al 2016, Shah et al 2017). While HS pathophysiology is still largely unknown, the benefit of tumour necrosis factor alpha (TNFα) blockade have been described in larger studies (Kimball et al 2016). Evidence of the efficacy of anti-IL1 treatment (Tzanetakou et al 2016) and of blocking IL-17A (Thorlacius et al 2017, Schuch et al 2018, Giuseppe et al 2018, Jørgensen et al 2018) or anti IL-23 treatment (Sharon et al 2012, Blok et al 2016) has also been observed in smaller studies and/or in case reports. More recently, investigational approaches using the oral PDE4 inhibitor apremilast (Weber et al 2017) or an anti-complement 5a compound (Kanni et al 2018) have been described.

The disease starts after puberty and women are more frequently affected than men (3:1). Risk factors include obesity and smoking. Although epidemiological prevalence estimates vary widely (0.03-4.3%; Jemec 2012, Jemec and Kimball 2015), and geographical differences exist, a prevalence of approximately 0.1-1% is accepted by the scientific community (Garg et al 2018).

The clinical manifestations of HS are heterogeneous, but the disease tends to manifest with chronic relapsing, deep, painful, inflammatory skin lesions, mostly inflammatory nodules and abscesses, leading to possible drainage and suppuration. Inflammatory lesions are complicated during disease progression by sinus tract formation and fistulization, and may lead to hypertrophic scarring with a possible impact on functional use.

HS is associated with pain, malodorous discharge from the wounds, and scarring, and does frequently have devastating psychosocial effects. HS is a profoundly debilitating disease with a high negative impact on quality of life (QoL), with multiple studies confirming that the impact is greater than that seen with other dermatologic diseases (Deckers and Kimball 2016). Patients with HS also often suffer from depression, social isolation, have impaired sexual health, and may have difficulty performing their work duties (Esmann and Jemec 2011, Fimmel and Zouboulis 2010 Januaryse et al 2017).

HS is difficult to treat. Official European treatment guidelines were only developed in 2015 and suggest that patients should be provided with adjuvant, medical and surgical therapy (Zouboulis et al 2015).

While topical antibiotics can be used for mild cases, long courses of multiple systemic antimicrobial therapies are preferred for moderate to severe HS, generally with tetracyclines or a combination of clindamycin and rifampicin, which can be followed by maintenance with chronic antibiotic treatment for months or even years (Bettoli et al 2016, Dessinioti et al 2016, Zouboulis et al 2015).

However, it is widely recognized that HS is a chronic inflammatory condition, not an infectious disease (Jemec 2012). Therefore, anti-inflammatory agents are an alternative and probably more appropriate approach than antibiotics or could be complementary to antibiotics. Over time, the consequence of chronic, recurrent, inadequately treated inflammation is irreversible fibrosis, which manifests as scarring and tunnels, or sinus tracts, which often do not respond to medical therapy. Once lasting anatomical changes occur, the only therapeutic option to reduce the volume of fibrotic tissue and improve functionality in the areas of affected skin is surgery (Andersen and Jemec 2017). One of the future treatment goals should be to reduce persistent scarring and to avoid surgery, which may be achieved by prevention of inflammatory lesions or may need a specific treatment.

In 2015, adalimumab (Humira®), a recombinant human monoclonal immunoglobulin G1 (IgG1) antibody to soluble and membrane bound TNF-α, received regulatory approval for the treatment of moderate to severe HS. Efficacy has been seen with adalimumab, with HISCR (Hidradenitis suppurativa clinical response) response rates over placebo of approximately 16% (41.8% adalimumab vs 26% placebo) and 31% (58.9% adalimumab vs 27.6% placebo) as reported in PIONEER I and II studies, respectively (Kimball et al 2016). As captured in the adalimumab labels, adalimumab is associated with an increased safety risk for serious infections including tuberculosis, invasive fungal infections and other opportunistic infections. An increased incidence of malignancies has also been reported with adalimumab.

There is, therefore, an unmet need for systemic therapies that effectively reduce inflammation while having a favorable safety profile for patients suffering from moderate to severe HS.

### SUMMARY OF THE DISCLOSURE

In HS, both cytokines IL-1β and IL-18 are upregulated (Kelly et al. 2015) and may thus play a role in its pathogenesis. IL-1β signature is present in HS lesions and can be reversed by application of an IL-1 receptor antagonist (Witte-Handel et al. 2019). Anakinra, a recombinant IL-1R antagonist, has show promising clinical efficacy results versus placebo in a small study (Tzanetakou et al. 2016), while case reports have confirmed these findings (Leslie et al 2014, Zarchi et al 2013, André et al 2019). Case reports have shown that IL-1β blockade using the anti IL-1β antibody canakinumab alone may be of benefit in moderate to severe HS (Houriet et al 2017) or to associated syndromes such as PASH (pyoderma gangraenosum, acne and suppurative hidradenitis (Jaeger et al 2013). However, some observed failures in HS with canakinumab (Sun et al 2017, Tekin et al 2017) and with anakinra (van der Zee and Prens 2013, Russo and Alikhan 2016) hint possibly to the fact that only subpopulations may be responsive to anti-IL-1 blockade or that this blockade alone may not be sufficient to achieve results in most patients.

Accordingly, it is an object of the present disclosure to target both inflammasome effector cytokines, IL-1β and IL-18, which therefore may provide superior clinical efficacy in (auto)-inflammatory conditions or where both IL-1β and IL-18 independently contribute to disease pathophysiology, such as HS. It is a further object of the present disclosure that a bispecific anti-IL-13/18 antagonist, may rapidly neutralize inflammasome dependent as well as inflammasome independent sources of IL-1β and IL-18.

Thus, any antagonist capable of inhibiting both IL-1β and IL-18, such as a bispecific anti-IL-1β/18 antibody or fragments thereof, could be suitable for the treatment of HS

Described herein is a bispecific antibody or functional fragments thereof targeting both IL-1β and IL-18 simultaneously, for use in preventing or treating Hidradenitis suppurativa (HS) in a subject. In a preferred embodiment, the anti-IL-1β/18 antibody comprises a heavy chain CH3 mutation that silences ADCC activity, such as the so-called LALA mutant comprising L234A and L235A mutation in the IgG1 Fc amino acid sequence. In a preferred embodiment, the anti-IL-1β/18 antibody comprises complementary heavy chain CH3 knob-in-hole mutations, e.g., (according to EU numbering) a first heavy chain having a S354C and T366W, knob-type mutation, and a second heavy chain having a Y349C, T366S, L368A, Y407V, hole-type mutation. In a preferred embodiment, the anti-IL-1β/18 antibody comprises a first light chain that preferentially associates with the first heavy chain and comprises a kappa light chain, e.g., Vk6, and a second light chain that preferentially associates with the second heavy chain and comprises a lambda light chain, e.g., Vλ1. In a yet more preferred embodiment, the anti-IL-1β/18 antibody comprises a combination of i) one or more ADCC silencing mutations (e.g., LALA), ii) one or more knob-in-hole heavy chain modifications, iii) and/or kappa and lambda light chains. In a yet more preferred embodiment, the anti-IL-1β/18 antibody comprises all of the foregoing features i)-iii), preferably wherein the antibody comprises a Vk6 and a VΔ1 light chain.

Described herein are also methods of preventing or treating Hidradenitis suppurativa (HS) by administering to a subject in need thereof a therapeutically effective amount of a bispecific antibody targeting both IL-1β and IL-18 simultaneously.

Further provided herein are specific dosing regimens for the methods or use of a bispecific antibody targeting both IL-1β and IL-18 simultaneously (e.g., bbmAb1) described herein.

Additionally described herein are pharmaceutical combinations and pharmaceutical compositions comprising a) a bispecific antibody targeting both IL-1β and IL-18 simultaneously (e.g., bbmAb1), and b) at least one further therapeutic agent, optionally in the presence of a pharmaceutically acceptable carrier, for use in the treatment or prevention of HS. Further features and advantages of the described methods and uses will become apparent from the following detailed description

In a first aspect the disclosure relates to a method for the treatment or prevention of HS in a subject in need thereof, comprising administering to said subject a therapeutically effective amount of a bispecific antibody, wherein the antibody comprises
a. a first part which is an immunoglobulin with a first variable light chain of (VL1) and a first variable heavy chain (VH1), that binds specifically to a IL1β, and a first constant heavy chain (CH1) with a hetero-dimerization modification, and
b. a second part which is an immunoglobulin with a second variable light chain (VL2) and a second variable heavy chain (VH2), that binds specifically to IL-18 and a second constant heavy chain (CH2) with a hetero-dimerization modification which is complementary to the hetero-dimerization modification of the first constant heavy chain.

In a second aspect the disclosure relates to a method for slowing, arresting, or reducing the development of HS in a subject in need thereof, comprising administering to said subject a therapeutically effective amount of a bispecific antibody, wherein the antibody comprises
a. a first part which is an immunoglobulin with a first variable light chain of (VL1) and a first variable heavy chain (VH1), that binds specifically to a IL1β, and a first constant heavy chain (CH1) with a hetero-dimerization modification, and
b. a second part which is an immunoglobulin with a second variable light chain (VL2) and a second variable heavy chain (VH2), that binds specifically to IL-18 and a second constant heavy chain (CH2) with a hetero-dimerization modification which is complementary to the hetero-dimerization modification of the first constant heavy chain. In a particular embodiment of the first and second aspects of the disclosure, the first and second constant heavy chains of the bispecific antibody are human IgA, IgD, IgE, IgG, or IgM, preferably IgD, IgE or IgG, such as human IgG1, IgG2, IgG3, or IgG4, preferably IgG1.

In another embodiment of the disclosure the first and second constant heavy chains of the bispecific antibody are IgG1, and
a. the first constant heavy chain has point mutations generating a knob structure and the second constant heavy has point mutations generating a hole structure, or
b. the first constant heavy chain has point mutations generating a hole structure and the second constant heavy has point mutations generating a knob structure, and optionally
c. the first and second constant heavy chains have mutations resulting in a disulfide bridge.

In a specifically preferred embodiment of the disclosure, including the first and second aspect, the first immunoglobulin VH1 domain of the bispecific antibody comprises:
i. hypervariable regions CDR1, CDR2 and CDR3, said CDR1 having the amino acid sequence SEQ ID NO:76, said CDR2 having the amino acid sequence SEQ ID NO:77, and said CDR3 having the amino acid sequence SEQ ID NO:78; or
ii. hypervariable regions CDR1, CDR2 and CDR3, said CDR1 having the amino acid sequence SEQ ID NO:79, said CDR2 having the amino acid sequence SEQ ID NO:80, and said CDR3 having the amino acid sequence SEQ ID NO:81; and the first immunoglobulin VL1 domain of the bispecific antibody comprises:
iii. hypervariable regions CDR1, CDR2 and CDR3, said CDR1 having the amino acid sequence SEQ ID NO:92, said CDR2 having the amino acid sequence SEQ ID NO:93, and said CDR3 having the amino acid sequence SEQ ID NO:94 or
iv. hypervariable regions CDR1, CDR2 and CDR3, said CDR1 having the amino acid sequence SEQ ID NO:95, said CDR2 having the amino acid sequence SEQ ID NO:96, and said CDR3 having the amino acid sequence SEQ ID NO:97; and the second immunoglobulin VH2 domain of the bispecific antibody comprises:
v. hypervariable regions CDR1, CDR2 and CDR3, said CDR1 having the amino acid sequence SEQ ID NO:44, said CDR2 having the amino acid sequence SEQ ID NO:45, and said CDR3 having the amino acid sequence SEQ ID NO:46; or
vi. hypervariable regions CDR1, CDR2 and CDR3, said CDR1 having the amino acid sequence SEQ ID NO:47, said CDR2 having the amino acid sequence SEQ ID NO:48, and said CDR3 having the amino acid sequence SEQ ID NO:49; and the second immunoglobulin VL2 domain of the bispecific antibody comprises:
vii. hypervariable regions CDR1, CDR2 and CDR3, said CDR1 having the amino acid sequence SEQ ID NO:60, said CDR2 having the amino acid sequence SEQ ID NO:61, and said CDR3 having the amino acid sequence SEQ ID NO:62 or
viii. hypervariable regions CDR1, CDR2 and CDR3, said CDR1 having the amino acid sequence SEQ ID NO:63, said CDR2 having the amino acid sequence SEQ ID NO:64, and said CDR3 having the amino acid sequence SEQ ID NO:65.

In another preferred embodiment of the disclosure, the antibody used in the methods, compositions, and uses described herein comprises:
a. the first immunoglobulin VH1 domain of amino acid sequence SEQ ID NO: 85,
b. the first immunoglobulin VL1 domain of amino acid sequence SEQ ID NO: 101,
c. the second immunoglobulin VH2 domain of amino acid sequence SEQ ID NO: 53, and
d. the second immunoglobulin VL2 domain of amino acid sequence SEQ ID NO: 69.

In another preferred embodiment of the disclosure, the antibody used in the methods, compositions, or uses described herein comprises
e. the first immunoglobulin heavy chain of amino acid sequence SEQ ID NO: 87,
f. the first immunoglobulin light chain of amino acid sequence SEQ ID NO: 103,
g. the second immunoglobulin heavy chain of amino acid sequence SEQ ID NO: 55, and
h. the second immunoglobulin light chain of amino acid sequence SEQ ID NO: 71.

In a specifically preferred embodiment of the disclosure, the treated subject has HS.

In a third aspect, the disclosure relates to a bispecific antibody comprising
a. a first part which is an immunoglobulin with a first variable light chain of (VL1) and a first variable heavy chain (VH1), that binds specifically to a IL1β, and a first constant heavy chain (CH1) with a hetero-dimerization modification, and
b. a second part which is an immunoglobulin with a second variable light chain (VL2) and a second variable heavy chain (VH2), that binds specifically to IL-18 and a second constant heavy chain (CH2) with a hetero-dimerization modification which is complementary to the hetero-dimerization modification of the first constant heavy chain, for use in the treatment or prevention of HS in a subject in need thereof.

In a fourth aspect the disclosure relates to method and treatments of the first, second and third aspect, wherein about 1 mg/kg to about 35 mg/kg of the bispecific antibody targeting both IL-1β and IL-18 simultaneously is administered to the subject. In a preferred embodiment of the fourth aspect, about 10 mg/kg of the bispecific antibody are administered to the treated subject.

In one aspect of the disclosure the bispecific antibody targeting both IL-1β and IL-18 simultaneously is administered to the subject intravenously or subcutaneously. In one embodiment, the route of administration is a combination of subcutaneous or intravenous, e.g., an intravenous loading dose followed by a subcutaneous maintenance dose.

In an embodiment, the bispecific antibody targeting both IL-1β and IL-18 simultaneously is administered, e.g., intravenously, to the treated subject at a dose of about 1.5 mg to about 15 mg active ingredient per kilogram of a human subject. In an embodiment, the bispecific antibody targeting both IL-1β and IL-18 simultaneously is administered, e.g., intravenously, to the treated subject at a dose of about 2, 4, or 5 mg active ingredient per kilogram of a human subject. In an embodiment, the bispecific antibody targeting both IL-1β and IL-18 simultaneously is administered, e.g., intravenously, to the treated subject at a dose of about 4 mg active ingredient per kilogram of a human subject. The dose, e.g., between about 1.5 to about 15 mg active ingredient per kilogram of a human subject, may be given weekly, every two weeks or every four weeks, or a combination thereof. In a preferred embodiment, the dose, e.g., between about 1.5 to about 15 mg active ingredient per kilogram of a human subject, may be every two weeks or every four weeks, or a combination thereof.

In one embodiment, the dose is about 75 mg to about 600 mg active ingredient, preferably about 150 mg to about 300 mg active ingredient, or is about 150 mg or 300 mg active ingredient, preferably 300 mg active ingredient. The dose may be given weekly, every two weeks or every four weeks, or a combination thereof. In preferred embodiments, a fixed dose (e.g., a non-weight based dose) is administered subcutaneously or intramuscularly, preferably subcutaneously.

In one embodiment, the dose is about 300 mg active ingredient.

In one preferred embodiment, the dose is 150 mg active ingredient. In another preferred embodiment, the dose is 300 mg active ingredient. In yet another preferred embodiment, the dose is 600 mg active ingredient. In yet another embodiment, the dose is from about 150 mg active ingredient to about 600 mg active ingredient.

In one embodiment, the antibody is administered through a loading dosing and a maintenance dosing. In one embodiment, the loading dosing is administered via one or more intravenous injections of a first dose and the maintenance dosing is administered via subcutaneous injections of a second dose. In one embodiment, the loading dosing is administered via subcutaneous injections of a first dose and the maintenance dosing is administered via subcutaneous injections of a second dose.

In one embodiment, the loading dosing is administered via subcutaneous injections of a first dose regimen and the maintenance dosing is administered via subcutaneous injections of a second dose regimen. The first dose regimen amount may be the same as the second dose regimen amount or higher than the second dose regimen amount. The first dose regimen period may be the same as the second dose regimen period or more frequent than the second dose regimen period.

In one embodiment, the first dose is between about 150 mg and about 600 mg active ingredient, such as about 300 mg active ingredient and the second dose is between about 150 mg and about 600 mg active ingredient, such as about 300 mg active ingredient.

In one embodiment, the first dose is 150 mg, 300 mg or 600 mg active ingredient and the second dose is 150 mg, 300 mg or 600 mg active ingredient. In one embodiment, the first dose is 300 mg active ingredient and the second dose is 300 mg active ingredient.

In one embodiment, the loading dosing comprises at least two administrations and the maintenance dosing consists of weekly (Q1W), preferably biweekly (Q2W), or monthly (Q4W) administrations. In one embodiment, the loading dosing consists of two administrations. In another embodiment, the loading dosing consists of three administrations. In another embodiment, the loading dosing consists of four administrations. In some cases, the loading dosing is a biweekly loading dose consisting of 3 doses from day 1 to day 29.

In one embodiment, the loading dosing comprises at least two subcutaneous injections, preferably three subcutaneous injections, and the maintenance dosing consists of weekly (Q1W), biweekly (Q2W), or preferably monthly (Q4W) subcutaneous injections. In one embodiment, the loading dosing consists of two subcutaneous injections. In another embodiment, the loading dosing consists of three subcutaneous injections, e.g., 3 biweekly (Q2W) 150 mg or 300 mg subcutaneous injections. In another embodiment, the loading dosing consists of four subcutaneous injections. In some cases, the loading dosing is a biweekly loading dose consisting of 3 subcutaneous injection doses from day 1 to day 29. In some cases, the maintenance dosing is a monthly (Q4W) maintenance dose comprising subcutaneous Q4W injection doses beginning on day 57, e.g., after three biweekly (Q2W) 150 mg or 300 mg subcutaneous injections. In some cases, the loading dosing is a biweekly loading dose consisting of 3 subcutaneous injection doses on day 1, day 15, and day 29, and the maintenance dosing is a monthly (Q4W) maintenance dose comprising subcutaneous Q4W injection doses beginning on day 57. In some cases, the loading dosing is a biweekly (e.g., 150 mg or 300 mg) loading dose consisting of 3 subcutaneous injection doses on day 1, day 15, and day 29, and the maintenance dosing is a monthly (Q4W) (e.g., 150 mg or 300 mg) maintenance dose comprising subcutaneous Q4W injection doses beginning on day 57.

In one embodiment, the loading dosing is a biweekly loading dose consisting of 3 doses from day 1 to day 29, wherein the dose amount is from about 1.5 mg to about 15 mg per kilogram active ingredient, e.g., wherein the loading doses are administered intravenously, subcutaneously, or a combination of intravenously or subcutaneously. In one embodiment, the loading dosing is a biweekly loading dose consisting of 3 doses from day 1 to day 29, wherein the dose amount is about 4 mg per kilogram active ingredient, e.g., wherein the loading doses are administered intravenously, subcutaneously, or a combination of intravenously or subcutaneously.

In one embodiment, the loading dosing is a biweekly loading dose consisting of 3 doses from day 1 to day 29, wherein the dose amount is about 150 mg or about 300 mg active ingredient, preferably 300 mg active ingredient, e.g., wherein the loading doses are administered subcutaneously, or a combination of intravenously or subcutaneously. In one embodiment, the loading dosing is a biweekly loading dose consisting of 3 doses from day 1 to day 29, wherein the dose amount is about 300 mg active ingredient wherein the loading doses are administered subcutaneously.

In some embodiments, the loading dose is a dose selected, or predicted, to achieve a plasma concentration of at least about 20 μg/mL to about 60 μg/mL during the loading dose period (e.g., within 1, 2, or 3 weeks or after 1, 2, or 3 loading dose injections. In some embodiments, the maintenance dose is a dose selected, or predicted, to provide a sustained plasma concentration of from about 20 μg/mL to about 60 μg/mL during the maintenance dosing period, or a substantial portion thereof (e.g., from at least about day 29 to about day 85).

In one embodiment, the subcutaneous injections of the loading dosing are different doses. In another embodiment, the subcutaneous injections of the loading dosing are the same dose.

The HS patient may be selected according to one of the following criteria:
the patient has moderate to severe HS;
the patient is an adult;
the patient is an adolescent;
prior to treatment with the IL-1β and IL-18 antagonist (e.g., bispecific antibody targeting both IL-1β and IL-18), the patient has an HS-PGA score of ≥3;
prior to treatment with the IL-1β and IL-18 antagonist (e.g., bispecific antibody targeting both IL-1β and IL-18), the patient has at least 3 inflammatory lesions; or
prior to treatment with the IL-1β and IL-18 antagonist (e.g., bispecific antibody targeting both IL-1β and IL-18), the patient does not have extensive scarring (<10 fistulas) as a result of HS.

In one embodiment, by week 16 of treatment the HS patient achieves at least one of the following:
a simplified HiSCR;
a reduction in HS flares;
a NRS30;
a reduction of ≤6 as measured by the DLQI; and/or
an improvement in DLQI.

In one embodiment, by week 16 of treatment, at least 40% of said patients achieve a simplified HiSCR; or at least 25% of said patients achieve an NRS30 response; or less than 15% of said patients experience an HS flare.

In one embodiment, the patient has at least one of the following as early as one week after the first dose of the IL-1β and IL-18 antagonist:
a rapid reduction in pain, as measured by VAS or NRS, and
a rapid reduction in CRP, as measured using a standard hsCRP assay.

In one embodiment, the patient achieves a sustained response 3 months after the end of the treatment, as measured by inflammatory lesion count, HS Clinical Response (HiSCR), Numerical Rating Scale (NRS), modified Sartorius HS score, Hidradenitis Suppurativa-Physician Global Assessment (HS-PGA), or Dermatology Life Quality Index (DLQI).

In one embodiment, the patient achieves a sustained response 3 months after the end of treatment, as measured by the simplified HiSCR (sHISCR).

According to a second aspect, a method of treating HS in a human subject is provided, comprising administering a therapeutically effective dose of an IL-1β and IL-18 antagonist to said subject.

In another embodiment of the disclosure, the bispecific antibody targeting both IL-1β and IL-18 is administered in combination with at least one further therapeutic agent.

In another aspect the disclosure relates to the use of a bispecific antibody targeting both IL-1β and IL-18 (e.g. bbmAb1) for the manufacture of a medicament for treating, slowing, arresting, or reducing the development of HS in a subject.

In one embodiment, provided herein is a method of treatment of HS in a subject in need thereof, comprising administering an effective amount a bispecific antibody targeting both IL-1β and IL-18, e.g. bbmAb1. In one embodiment, provided herein is a bispecific antibody targeting both IL-1β and IL-18, e.g. bbmAb1, for use in treating HS in a subject in need thereof. In some embodiments, provided herein is the use of a bispecific antibody targeting both IL-1β and IL-18, e.g. bbmAb1, for the manufacture of a medicament for the treatment of HS.

In one embodiment, provided herein is a method for treating, slowing, arresting, or reducing the development of HS in a subject in need thereof, comprising administering to said subject a therapeutically effective amount of a bispecific antibody targeting both IL-1β and IL-18, e.g. bbmAb1. In one embodiment, the therapeutically effective amount of is an amount of a bispecific antibody comprising
a. a first immunoglobulin VH1 domain of the bispecific antibody comprises the amino acid sequence SEQ ID NO: 85,
b. a first immunoglobulin VL1 domain of the bispecific antibody comprises the amino acid sequence SEQ ID NO: 101,
c. a second immunoglobulin VH2 domain of the bispecific antibody comprises the amino acid sequence SEQ ID NO: 53, and
d. a second immunoglobulin VL2 domain of the bispecific antibody comprises the amino acid sequence SEQ ID NO: 69.

According to another aspect, the present disclosure provides liquid pharmaceutical composition, a method or use of a liquid pharmaceutical composition comprising a bispecific antibody that specifically targets both IL-18 and IL-1β, wherein the liquid pharmaceutical formulation comprises a buffer, a stabilizer and a solubilizer. In one embodiment, the disclosure provides or further provides a means for administering or subcutaneously administering the bispecific antibody to a patient having HS. In one embodiment, the use is for the manufacture of a medicament for the treatment of HS.

In a specifically preferred embodiment of the disclosure, the first immunoglobulin VH1 domain of the bispecific antibody of the liquid pharmaceutical composition comprises:
i. hypervariable regions CDR1, CDR2 and CDR3, said CDR1 having the amino acid sequence SEQ ID NO:76, said CDR2 having the amino acid sequence SEQ ID NO:77, and said CDR3 having the amino acid sequence SEQ ID NO:78; or
ii. hypervariable regions CDR1, CDR2 and CDR3, said CDR1 having the amino acid sequence SEQ ID NO:79, said CDR2 having the amino acid sequence SEQ ID NO:80, and said CDR3 having the amino acid sequence SEQ ID NO:81; and the first immunoglobulin VL1 domain of the bispecific antibody comprises:
iii. hypervariable regions CDR1, CDR2 and CDR3, said CDR1 having the amino acid sequence SEQ ID NO:92, said CDR2 having the amino acid sequence SEQ ID NO:93, and said CDR3 having the amino acid sequence SEQ ID NO:94 or
iv. hypervariable regions CDR1, CDR2 and CDR3, said CDR1 having the amino acid sequence SEQ ID NO:95, said CDR2 having the amino acid sequence SEQ ID NO:96, and said CDR3 having the amino acid sequence SEQ ID NO:97; and the second immunoglobulin VH2 domain of the bispecific antibody comprises:
v. hypervariable regions CDR1, CDR2 and CDR3, said CDR1 having the amino acid sequence SEQ ID NO:44, said CDR2 having the amino acid sequence SEQ ID NO:45, and said CDR3 having the amino acid sequence SEQ ID NO:46; or
vi. hypervariable regions CDR1, CDR2 and CDR3, said CDR1 having the amino acid sequence SEQ ID NO:47, said CDR2 having the amino acid sequence SEQ ID NO:48, and said CDR3 having the amino acid sequence SEQ ID NO:49; and the second immunoglobulin VL2 domain of the bispecific antibody comprises:
vii. hypervariable regions CDR1, CDR2 and CDR3, said CDR1 having the amino acid sequence SEQ ID NO:60, said CDR2 having the amino acid sequence SEQ ID NO:61, and said CDR3 having the amino acid sequence SEQ ID NO:62 or
viii. hypervariable regions CDR1, CDR2 and CDR3, said CDR1 having the amino acid sequence SEQ ID NO:63, said CDR2 having the amino acid sequence SEQ ID NO:64, and said CDR3 having the amino acid sequence SEQ ID NO:65.

In another preferred embodiment of the disclosure, the antibody of the liquid pharmaceutical composition described herein comprises:
a. the first immunoglobulin VH1 domain of amino acid sequence SEQ ID NO: 85,
b. the first immunoglobulin VL1 domain of amino acid sequence SEQ ID NO: 101,
c. the second immunoglobulin VH2 domain of amino acid sequence SEQ ID NO: 53, and
d. the second immunoglobulin VL2 domain of amino acid sequence SEQ ID NO: 69.

In another preferred embodiment of the disclosure, the antibody of the liquid pharmaceutical composition described herein comprises
e. the first immunoglobulin heavy chain of amino acid sequence SEQ ID NO: 87,
f. the first immunoglobulin light chain of amino acid sequence SEQ ID NO: 103,
g. the second immunoglobulin heavy chain of amino acid sequence SEQ ID NO: 55, and
h. the second immunoglobulin light chain of amino acid sequence SEQ ID NO: 71.

In some embodiments, the liquid pharmaceutical composition comprises about 50 mg/mL to about 120 mg/mL bispecific antibody (e.g., bbmAb1), a buffer, a stabilizer (e.g., non-ionic stabilizer, such as a sugar), and a surfactant (e.g., polysorbate 20 or polysorbate 80). In some embodiments, the liquid pharmaceutical composition comprises about 50 mg/mL to about 120 mg/mL bispecific antibody in a buffer at a pH of from about 5.5 to about 6.5, preferably at a pH of from about 5.5 to about 6.0, preferably at a pH of about 6.0.

In some embodiments, the liquid pharmaceutical composition comprises about 100 mg/mL bispecific antibody (e.g., bbmAb1), a buffer, a stabilizer (e.g., non-ionic stabilizer, such as a sugar), and a surfactant (e.g., polysorbate 20 or polysorbate 80). In some embodiments, the liquid pharmaceutical composition comprises about 50 mg/mL to about 120 mg/mL bispecific antibody (e.g., bbmAb1), from about 1 mM to about 50 mM histidine/histidine-chloride, from about 50 mM to about 400 mM sugar (e.g., sucrose, trehalose, or mannitol), from about 0.01% to about 0.5% surfactant (e.g., polysorbate, such as polysorbate 20), at a pH of from about 5.5 to about 6.5, preferably from about 5.5 to about 6.0, more preferably about 6.0. In some cases, the liquid pharmaceutical composition comprises about 50 mg/mL to about 120 mg/mL bbmAb1 (preferably about 100 mg/mL bbmAb1), about 20 mM histidine/histidine-chloride, about 220 mM sucrose, about 0.04% polysorbate 20 (w/v), at a pH of about 6.0.

### BRIEF DESCRIPTION OF THE FIGURES

FIG. 1 is a graphic illustration of the mRNA levels of the AIM2, NLRC4, NLRP7 and NLRP3 inflammasomes in HS lesions compared to levels of non-lesional or healthy tissue.

FIG. 2 is a TIBCO spotfire heat map representation of the average expression level of genes/members of the IL-18, IL-12 and IL-1β (alone or in combination) or CRS signaling signatures in biopsies of healthy skin or lesional, peri-lesional and lesional skin from HS patients. The identification of the genes/members of respective IL-18, IL-12 and IL-1β signaling signatures was done in a separate experiment using PBMCs from healthy donors stimulated for 6 hours (limited to genes induced by the respective cytokine=UP) or taken from a publication (canakinumab response signature CRS; Brachat et al 2017).

FIG. 3 shows the effects of either of bbmAb1 (100 μg/ml) and Adalimumab (100 μg/ml) incubation compared to untreated controls on the levels of the analytes IFNγ, TNFα, IL-1β and IL-2 produced into the supernatant of HS skin biopsies after 24 hours of in vitro culture. Each dot represents a single biopsy. Left most column for each analyte is untreated control, middle column for each analyte is bbmAb1, and right most column for each analyte is Adalimumab. Shown are values from n=54, n=36 and n=26 individual biopsies for IFNγ, IL-1β and TNFα and n=43, n=20 and n=10 biopsies for IL-2 obtained from 9 different HS patients.

FIG. 4 is a graphic illustration of a prediction of the effects of bbmAb1 dosing on IL-1β levels. The dashed line refers to the lower limit of quantitation. The prediction is modeled assuming a 300 mg subcutaneous dose on day 1, 15, 29, 57, and 85.

FIG. 5 is a graphic illustration of a prediction of the effects of bbmAb1 dosing on IL-18 levels relative to the change in baseline in healthy control subjects. The dashed line refers to the levels in healthy control subjects. The prediction is modeled assuming a 300 mg subcutaneous dose on day 1, 15, 29, 57, and 85.

FIG. 6 is a graphic illustration of a prediction of the pharmacokinetics of subcutaenously administered bbmAb1 in comparison to a single dose of 10 mg/kg i.v. The prediction is modeled assuming a 300 mg subcutaneous dose on day 1, 15, 29, 57, and 85.

### DETAILED DESCRIPTION OF THE DISCLOSURE

Described herein are IgG-like bispecific monoclonal antibodies (e.g., bbmAb1) containing a heterodimeric Fc part that simultaneously neutralizes the key inflammasome effector cytokines interleukin-1 beta (IL-1β) and interleukin-18 (IL-18). In certain aspects, the bispecific antibodies have picomolar (pM) affinities to both cytokines and inhibit IL-1β and IL-18 signaling in cellular in vitro assays at sub-nanomolar (nM) concentrations. IL-1β and IL-18 are the two pro-inflammatory cytokines secreted after inflammasome activation in response to the recognition of damage associated molecular patterns (DAMPs) and pathogen associated molecular patterns (PAMPs). As described herein, the combined inhibition of IL-1β and IL-18 can result in a more effective down-modulation of the inflammasome-driven pro-inflammatory responses compared to the inhibition of either cytokine alone.

Thus, any antagonist capable of simultaneously blocking IL-1β and IL-18 activity, such as a bispecific anti-IL-1β and anti-IL-18 antibody with silenced ADCC activity, could be suitable for the treatment of HS.

Without wishing to be bound by theory, the inventors have identified that a loading regimen may be beneficial at start of treatment to at least partially, or fully, saturate bispecific antibody binding sites (IL-1β and/or IL-18) in these patients in conditions where IL-1β and/or IL-18 levels have been enhanced, requiring higher doses or a more frequent regimen at start of treatment. Thus, with a loading dosing regimen providing at start of treatment (2 to 3 weeks) rapid saturation of antigen, followed by a maintenance dosing regimen providing, throughout the entire treatment period, sustained therapeutic plasma concentrations, in situations where IL-1β and/or IL-18 expression in affected tissues would be enhanced (severity of the condition), is considered for a therapeutic effect.

The appropriate dosage will vary depending upon, for example, the particular bispecific IL-1β and IL-18 antagonist, e.g. a bispecific anti-IL-1β and anti-IL-18 antibody or antigen binding fragment thereof (e.g., bbmAb1) to be employed, the subject of treatment, the mode of administration and the nature and severity of the condition being treated, and on the nature of prior treatments that the patient has undergone. Ultimately, the attending health care provider will decide the amount of the bispecific IL-1β and IL-18 antagonist with which to treat each individual patient. In some embodiments, the attending health care provider may administer low or even single doses of the bispecific IL-1β and IL-18 antagonist and observe the patient's response. In other embodiments, the initial dose(s) of bispecific IL-1β and IL-18 antagonist administered to a patient are high, and then are titrated downward until signs of relapse occur. Larger doses of the bispecific IL-1β and IL-18 antagonist may be administered until the optimal therapeutic effect is obtained for the patient, and the dosage is not generally increased further.

In one embodiment, the disclosure relates to a bispecific anti-IL-1β and anti-IL-18 antibody or antigen binding fragment thereof for use according to any of the aspects or embodiments of the disclosure as described above, wherein the loading dose and the maintenance dose of the bispecific anti-IL-1β and anti-IL-18 antibody or antigen binding fragment thereof (e.g., bbmAb1) is adjusted so that plasma or serum concentration of antibody is at a therapeutic level.

In practicing some of the methods of treatment or uses of the present disclosure, a therapeutically effective amount of a bispecific IL-1β and IL-18 antagonist, e.g. a bispecific anti-IL-1β and anti-IL-18 antibody or antigen binding fragment thereof (e.g., bbmAb1) is administered to a patient, e.g., a mammal (e.g., a human). While it is understood that the disclosed methods provide for treatment of HS patients using a bispecific IL-1β and IL-18 antagonist (e.g., bbmAb1), this does not preclude that, if the patient is to be ultimately treated with the antagonist, such therapy is necessarily a monotherapy. Indeed, if a patient is selected for treatment with a bispecific IL-1β and IL-18 antagonist, then the antagonist (e.g., bbmAb1) may be administered in accordance with the methods of the disclosure either alone or in combination with other agents and therapies.

It will be understood that regimen changes may be appropriate for certain HS patients, e.g., patients that display inadequate response to treatment with the bispecific IL-1β and IL-18 antagonist, e.g. a bispecific antibody or antigen binding fragment thereof (e.g., bbmAb1). Thus, administration may be more frequent than monthly dosing, e.g., bi-weekly dosing (every two weeks) or weekly dosing.

Some patients may benefit from a loading regimen (e.g., weekly administrations for several weeks [e.g., 1 to 4 weeks, e.g., dosing at weeks 0, 1, 2, and/or 3, such as 3 weeks, loading dosing regimen at Weeks 1, 2 and 3]) followed by a maintenance regimen starting e.g. at Week 5, 6, or 7, where the bispecific antibody (e.g., bbmAb1) may be administered weekly, bi-weekly or preferably every 4 weeks for at least several weeks.

For example, an appropriate regimen for bbmAb1 can be biweekly for several weeks [e.g., 1 to 5 weeks, e.g., dosing at weeks 1, 3, and 5] followed by a monthly Q4W maintenance regimen e.g. at Week 8 or 9, preferably 9 where the bispecific antibody (e.g., bbmAb1) may be administered for at least several weeks.

It will also be understood that administration may be less frequent than monthly dosing, e.g., dosing every 6 weeks, every 8 weeks (every two months), quarterly (every three months), etc.

It will be understood that dose escalation may be appropriate for certain HS patients, based on severity of the disease, e.g., patients that display inadequate response to treatment with the bispecific antagonists described herein, e.g. bbmAb1 or antigen-binding fragment thereof. Thus, subcutaneous (s.c.) dosages (loading or maintenance doses) may be greater than about 150 mg to about 900 mg s.c., e.g., about 75 mg, about 100 mg, about 125 mg, about 175 mg, about 200 mg, about 250 mg, about 350 mg, about 400 mg, about 450 mg, about 500 mg, about 600 mg, etc.; similarly, intravenous (i.v.) dosages may be greater than about 5 mg/kg or 10 mg/kg, e.g., about 6 mg/kg, 7 mg/kg, 8 mg/kg, 9 mg/kg, 11 mg/kg, 12 mg/kg, 15 mg/kg, 20 mg/kg, 25 mg/kg, 30 mg/kg, 35 mg/kg, etc. It will also be understood that dose reduction may also be appropriate for certain HS patients, e.g., patients that display adverse events or an adverse response to treatment with the bispecific antagonist (e.g. bbmAb1 or antigen-binding fragment thereof). Thus, dosages of the antagonist, may be less than about 150 mg to about 900 mg s.c., e.g., about 25 mg, about 50 mg, about 75 mg, about 100 mg, about 125 mg, about 175 mg, about 200 mg, about 250 mg, about 350 mg, about 400 mg, about 450 mg, about 500 mg, about 600 mg, etc.

In some embodiments, the bispecific antagonist, e.g. bbmAb1 or antigen-binding fragment thereof, may be administered to the patient at an initial dose of 300 mg delivered s.c., and the dose may be then adjusted to 150 mg or 600 mg delivered s.c. if needed, as determined by a physician.

The bispecific antibody or antigen-binding fragment thereof may be bbmAb1, a functional derivative thereof or a biosimilar thereof.

As herein defined, “unit dose” refers to a s.c. dose that can be comprised between about 75 mg to 900 mg, e.g. about 150 mg to about 600 mg, e.g. about 150 mg to about 600 mg, e.g. about 300 mg to about 600 mg, or a e.g. about 150 mg to about 300 mg. For example an unit s.c. dose is about 75 mg, about 150 mg, about 300 mg, about 350 mg, about 400 mg, about 450 mg, about 500 mg, about 550 mg, about 600 mg.

The present disclosure is inter alia based on the unexpected finding that certain antibodies that simultaneously neutralize IL-1β and IL-18 more potently attenuate IFN-γ (and other pro-inflammatory cytokines) production compared to single IL-1β or IL-18 neutralization alone, which is considered by the inventors to be an efficacious treatment of HS.

### 1. Definitions

For purposes of interpreting this specification, the following definitions will apply and whenever appropriate, terms used in the singular will also include the plural and vice versa. Additional definitions are set forth throughout the detailed description.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “comprising” encompasses “including” as well as “consisting,” e.g., a composition “comprising” X may consist exclusively of X or may include something additional, e.g., X+Y.

The term “IL-18” is synonym to IL-18 polypeptide, Interleukin-18 polypeptide, IFN-gamma inducing factor or Interferon-gamma-inducing-factor or INF-γ inducing factor. The term “IL-18” refers to human IL-18, unless another species is indicated. IL-18 is well known to a person skilled in the art, and for example obtainable from MBL® International Corporation under product reference #B001-5. Throughout this specification, the term IL-18 encompasses both pro-IL-18 (precursor of mature IL-18 prior protease cleavage) and mature IL-18 (post protease cleavage) interchangeably unless it is specified that the pro- or mature form is meant.

The term “IL-1β” or “IL-1b” is synonym to IL-13 polypeptide and Interleukin-1β polypeptide. The term “IL-1β” refers to human IL-1β unless another species is indicated. IL-1β is well known to a person skilled in the art, and for example obtainable from Sino Biological under product reference #10139-HNAE-5.

The term “antibody” refers to an intact immunoglobulin or a functional fragment thereof. Naturally occurring antibodies typically comprise a tetramer which is usually composed of at least two heavy (H) chains and at least two light (L) chains. Each heavy chain is comprised of a heavy chain variable region (abbreviated herein as VH) and a heavy chain constant region, usually comprised of three domains (CH1, CH2 ad CH3). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable region (abbreviated herein as VL) and a light chain constant region (CL). Light chain includes kappa (κ) chains and lambda (A) chains. The heavy and light chain variable region is typically responsible for antigen recognition, whilst the heavy and light chain constant region may mediate the binding of the immunoglobulin to host tissues or factors, including various cells of the immune system (e.g. effector cells) and the first component (Clq) of the classical complement system. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

The term “antigen-binding portion” of an antibody (or simply “antigen portion”), as used herein, refers to full length or one or more fragments of an antibody that retain the ability to specifically bind to IL-18 or IL-1β antigen. It has been shown that the antigen-binding function of an antibody can be performed by fragments of a full-length antibody. Examples of binding fragments encompassed within the term “antigen-binding portion” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature; 341:544-546), which consists of a VH domain; and an isolated complementarity determining region (CDR).

Furthermore, although the two domains of the Fv fragment, VL and VH, are coded for by separate genes, they can be joined, using recombinant methods, by a flexible linker that enables them to be made as a single protein chain in which the VL and VH regions pair to form monovalent molecules (known as single chain Fv (scFv); see e.g. Bird et al., (1988) Science 242:423-426; and Huston et al., (1988) Proc Natl Acad Sc;. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

The term “isolated” means throughout this specification, that the immunoglobulin, antibody or polynucleotide, as the case may be, exists in a physical milieu distinct from that in which it may occur in nature.

Throughout this specification, complementarity determining regions (“CDR”) are defined according to the Kabat definition unless specified that the CDR are defined according to another definition. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

By convention, the CDR regions in the heavy chain are typically referred to as H-CDR1, H-CDR2 and H-CDR3 and in the light chain as L-CDR1, LCDR2 and L-CDR3. They are numbered sequentially in the direction from the amino terminus to the carboxy terminus.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g. human germline sequences, or mutated versions of human germline sequences or antibody containing consensus framework sequences derived from human framework sequences analysis, for example, as described in Knappik, et al., (2000) J Mol Biol; 296:57-86).

The human antibodies of the invention may include amino acid residues not encoded by human sequences (e.g. mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences.

The term “recombinant human antibody”, as used herein, includes all human antibodies that are prepared, expressed, created or isolated by recombinant means, such as antibodies isolated from an animal (e.g. a mouse) that is transgenic or transchromosomal for human immunoglobulin genes or a hybridoma prepared therefrom, antibodies isolated from a host cell transformed to express the human antibody, e.g. from a transfectoma, antibodies isolated from a recombinant, combinatorial human antibody library, and antibodies prepared, expressed, created or isolated by any other means that involve splicing of all or a portion of a human immunoglobulin gene. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The phrases “an antibody recognizing an antigen” and “an antibody specific for an antigen” are used interchangeably herein with the term “an antibody which binds specifically to an antigen”.

As used herein, a binding molecule that “specifically binds to IL-18″is intended to refer to a binding molecule that binds to human IL-18 with a K_{D} of a 100 nM or less, 10 nM or less, 1 nM or less.

As used herein, a binding molecule that “specifically binds to IL-1β” is intended to refer to a binding molecule that binds to human IL-1β with a K_{D} of a 100 nM or less, 10 nM or less, 1 nM or less.

As used herein, the term “antagonist” is intended to refer to a binding molecule that inhibits the signalling activity in the presence of activating compound. For example, in the case of IL-18, an IL-18 antagonist would be a binding molecule inhibiting the signalling activity in the presence of IL-18 in a human cell assay such as IL-18 dependent Interferon-gamma (IFN-γ) production assay in human blood cells. Examples of an IL-18 dependent IFN-γ production assay in human blood cells are described in more details in the examples below.

The term bivalent bispecific antibody or bivalent bispecific antibodies refer to antibodies binding to two different targets, such as IL-18 and IL-1β. Such bivalent bispecific antibodies are also referred to herein as bispecific antibodies.

The bispecific antibodies are “hetero-dimers”, which means that one part comes from first antibody, specific for a first target, and another part comes from a second antibody, specific for a second target. A “hetero-dimerization modification” is a modification to one or both parts of the antibodies forming the hetero-dimeric bispecific antibody, intended to facilitate such formation. An example of hetero-dimerization modifications of the Fc domains of two IgG1 parts of antibodies intended to form a bispecific is a “knob” with a bulky amino acid (aa) side chain (S354C, T366W) in the first heavy chain and a “hole” with small aa side chains (Y349C, T366S, L368A, Y407V) were introduced in the second heavy chain as well as an additional disulfide bridge in the CH3 region connecting both heavy chains (Merchant et al., Nat. Biotechnol., 16:677-681 (1998), page 678, table 1).

The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, such as a Biacore® system.

As used herein, the term “affinity” refers to the strength of interaction between binding molecule and antigen at single antigenic sites.

As used herein, the term “high affinity” for an antibody refers to an antibody having a KD of 1 nM or less for a target antigen.

As used herein, the term “subject” includes any human subjects receiving the bispecific anti-IL-18 and IL-1β antagonist as presently described can present symptoms of or be at risk of HS.

As used herein, the term, “optimized nucleotide sequence” means that the nucleotide sequence has been altered to encode an amino acid sequence using codons that are preferred in the production cell or organism, generally a eukaryotic cell, for example, a cell of Pichia pastoris, a Chinese Hamster Ovary cell (CHO) or a human cell. The optimized nucleotide sequence is engineered to retain completely the amino acid sequence originally encoded by the starting nucleotide sequence, which is also known as the “parental” sequence. The optimized sequences herein have been engineered to have codons that are preferred in CHO mammalian cells; however optimized expression of these sequences in other eukaryotic cells is also envisioned herein.

The term “identity” refers to the similarity between at least two different sequences. This identity can be expressed as a percent identity and determined by standard alignment algorithms, for example, the Basic Local Alignment Tool (BLAST) (Altshul et al., (1990) J Mol Biol; 215:403-410); the algorithm of Needleman et al., (1970) J Mol Biol; 48:444-453 or the algorithm of Meyers et al., (1988) Comput Appl Biosci; 4:11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller, (1989) CABIOS; 4(1):1-17) which has been incorporated into the ALIGN program (version 2.0), using a PAM 120 weight residue table, a gap length penalty of 12 and a gap penalty of 4. The percent identity is usually calculated by comparing sequences of similar length.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” or “signaling activity” refers to a biochemical causal relationship generally initiated by a protein-protein interaction such as binding of a growth factor to a receptor, resulting in transmission of a signal from one portion of a cell to another portion of a cell. In general, the transmission involves specific phosphorylation of one or more tyrosine, serine, or threonine residues on one or more proteins in the series of reactions causing signal transduction. Penultimate processes typically include nuclear events, resulting in a change in gene expression.

The term “neutralises” and grammatical variations thereof means throughout this specification, that the biological activity of the target is reduced either totally or partially in the presence of the binding protein or antibody, as the case may be.

The term “nucleic acid” or “polynucleotide” refers to deoxyribonucleic acid (DNA) or ribonucleic acid (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994))

The nucleotide in the “polynucleotide” or “nucleic acid” may comprise modifications including base modifications such as bromouridine and inosine derivatives, ribose modification such as phosphorothioate, phosphorodithioate, phosphoroselenoate, phosphorodiselenoate, phosphoroanilothioate, phosphoraniladate and phosphoroamidate.

The term “vector” means any molecule or entity (e.g. nucleic acid, plasmid, bacteriophage or virus) that is suitable for transformation or transfection of a host cell and contains nucleic acid sequences that direct and/or control (in conjunction with the host cell) expression of one or more heterologous coding regions operatively linked thereto.

A “conservative variant” of a sequence encoding a binding molecule, an antibody or a fragment thereof refers to a sequence comprising conservative amino acid modifications. “Conservative amino acid modifications” are intended to refer to amino acid modifications that do not significantly affect or alter the binding characteristics of the antibody containing the amino acid sequence. Such conservative modifications include amino acid substitutions, additions and deletions. Conservative amino acid substitutions are ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine). Modifications can be introduced into a binding protein of the invention by standard techniques known in the art, such as site-directed mutagenesis and PCR-mediated mutagenesis. Conservative amino acid substitution can also encompass non-naturally occurring amino acid residues which are typically incorporated by chemical peptide synthesis rather than by synthesis in biological systems. Non-naturally occurring amino acids include, but are not limited to, peptidomimetic, reversed or inverted forms of amino acid moieties.

The term “epitope” is the part of an antigen that is recognized by the immune system, such as an antibody or a fragment thereof. Within the present specification, the term “epitope” is used interchangeably for both conformational epitopes and linear epitopes. A conformational epitope is composed of discontinuous sections of the antigen's amino acid sequence, whilst a linear epitope is formed by a continuous sequence of amino acids from the antigen.

A human antibody or a fragment thereof can comprise heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest.

A human antibody or fragment thereof that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutation.

However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g. murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Human antibodies may be produced by a number of methods known to those of skill in the art. Human antibodies can be made by the hybridoma method using human myeloma or mouse-human heteromyeloma cells lines (Kozbor, J Immunol; (1984) 133:3001; Brodeur, Monoclonal Isolated Antibody Production Techniques and Applications, pp51-63, Marcel Dekker Inc, 1987). Alternative methods include the use of phage libraries or transgenic mice both of which utilize human variable region repertories (Winter G; (1994) Annu Rev Immunol 12:433-455, Green LL, (1999) J Immunol Methods 231:11-23).

Several strains of transgenic mice are now available wherein their mouse immunoglobulin loci has been replaced with human immunoglobulin gene segments (Tomizuka K, (2000) Proc Natl Acad Sci, 97:722-727; Fishwild D M (1996) Nature Biotechnol 14:845-851; Mendez M J, (1997) Nature Genetics 15:146-156). Upon antigen challenge such mice are capable of producing a repertoire of human antibodies from which antibodies of interest can be selected. Of particular note is the Trimera™ system (Eren R et al, (1988) Immunology 93:154-161) where human lymphocytes are transplanted into irradiated mice, the Selected Lymphocyte Isolated antibody System (SLAM, Babcook et al, Proc Natl Acad Sci (1996) 93:7843-7848) where human (or other species) lymphocytes are effectively put through a massive pooled in vitro isolated antibody generation procedure followed by deconvoluted, limiting dilution and selection procedure and the Xenomouse™ (Abgenix Inc). An alternative approach is available from Morphotek Inc using the Morphodoma™ technology.

Phage display technology can be used to produce human antibodies and fragments thereof, (McCafferty; (1990) Nature, 348:552-553 and Griffiths A D et al (1994) EMBO 13:3245-3260). According to this technique, isolated antibody variable domain genes are cloned in frame into either a major or minor coat of protein gene of a filamentous bacteriophage such as M13 or fd and displayed (usually with the aid of a helper phage) as function isolated antibody fragments on the surface of the phage particle. Selections based on the function properties of the isolated antibody result in selection of the gene encoding the isolated antibody exhibiting these properties. The phage display technique can be used to select antigen specific antibodies from libraries made from human B cells taken from individuals afflicted with a disease or disorder or alternatively from unimmunized human donors (Marks; J Mol Bio (1991) 222:581-591,). Where an intact human isolated antibody is desired comprising an Fc domain it is necessary reclone the phage displayed derived fragment into a mammalian expression vectors comprising the desired constant regions and establishing stable expressing cell lines.

The technique of affinity maturation (Marks; Biotechnol (1992) 10:779-783) may be used to provide binding affinity wherein the affinity of the primary human isolated antibody is improved by sequentially replacing the H and L chain variable regions with naturally occurring variants and selecting on the basis of improved binding affinities. Variants of this technique such as ‘epitope imprinting’ are now also available (WO 93/06213; Waterhouse; Nucl Acids Res (1993) 21:2265-2266).

The term “pure” when used in the context of purified bispecific antibody relates to purity and identity of different bispecific antibody combinations and constructs after co-expression in selected cells under conditions wherein the cells express the bispecific antibody and after protein-A purification using an intact UPLC-MS mass screening approach. Pure or purity refers to the relative quantify of the formed hetero-and homodimer bbmAbs. Using the method of the invention correctly formed heterodimeric bbmAb1 can be observed with a relative purity of over 85% based on intact mass signal intensity.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development or progression of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. More specifically, the term “treating” the disease HS refers to treating the inflammatory lesions in HS patients (in numbers or quality or reducing their volume and size), and/or treating the abscesses and inflammatory nodules and/or draining fistulae in HS patients, and/or decreasing the amount of scarring and/or relieving the functional limitations associated with scarring. Treating the disease HS also refers to alleviating the pain, fatigue and/or itching associated with HS, reducing pus release and reducing the odor associated with pus release, and/or improving the quality of life and/or reducing the work impairment for HS patients.

As used herein, the term “prevention” refers delaying the onset or development or progression of the disease or disorder. More specifically, the term “preventing” the disease HS refers to preventing HS flares and or new lesions to appear; preventing scarring and preventing functional limitations associated with scarring and/or in particular preventing surgical interventions for HS.

As used herein, a subject is “in need of” a treatment if such subject would benefit biologically, medically or in quality of life from such treatment.

As used herein, a “therapeutically effective amount” refers to an amount of bispecific antibody targeting both IL-1β and IL-18 simultaneously (e.g., bbmAb1) or antigen binding fragment thereof, that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., bbmAb1) administered alone, the term refers to that ingredient alone. When applied to a combination, the term refers to combined amounts of the active ingredients that result in the therapeutic effect, whether administered in combination, serially or simultaneously.

The phrase “therapeutic regimen” means the regimen used to treat an illness, e.g., the dosing protocol used during the treatment of HS. A therapeutic regimen may include an loading regimen (or loading dosing), followed by a maintenance regimen (or maintenance dosing).

The phrase “loading regimen” or “loading period” refers to a treatment regimen (or the portion of a treatment regimen) that is used for the initial treatment of a disease. In some embodiments, the disclosed methods, uses, kits, processes and regimens (e.g., methods of treating HS) employ a loading regimen (or loading dosing). In some cases, the loading period is the period until maximum efficacy is reached. The general goal of a loading regimen is to provide a high level of drug to a patient during the initial period of a treatment regimen. A loading regimen may include administering a greater dose of the drug than a physician would employ during maintenance regimen, administering a drug more frequently than a physician would administer the drug during a maintenance regimen, or both. Dose escalation may occur during or after the loading regimen.

The phrase “maintenance regimen” or “maintenance period” refers to a treatment regimen (or the portion of a treatment regimen) that is used for the maintenance of a patient during treatment of an illness, e.g., to keep the patient in remission for long periods of time (months or years) following the loading regimen or period. In some embodiments, the disclosed methods, uses and regimens employ a maintenance regimen. A maintenance regimen may employ continuous therapy (e.g., administering a drug at a regular intervals, e.g., weekly, bi-weekly or monthly (every 4 weeks), yearly, etc.) or intermittent therapy (e.g., interrupted treatment, intermittent treatment, treatment at relapse, or treatment upon achievement of a particular predetermined criteria [e.g., pain, disease manifestation, etc.]). Dose escalation may occur during a maintenance regimen.

The phrase “means for administering” is used to indicate any available implement for systemically administering a drug to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

2. IL-18 antibody

Particularly preferred IL-18 antibodies or antigen-binding fragments thereof used in the disclosed methods are human antibodies.

For ease of reference, the amino acid sequences of the hypervariable regions of a specific IL-18 antibody, called mAb1, based on the Kabat definition and the Chothia definition, as well as the VL and VH domains and full heavy and light chains are provided in Table 1, below.

TABLE 1
Amino acid sequences of the hypervariable regions (CDRs), variable
domains (VH and VL) and full chains of mAb1. The DNA encoding
the VL of mAb1 is set forth in SEQ ID NO: 18. The DNA encoding
the VH of mAb1 is set forth in SEQ ID NO: 8.
mAb1 heavy chain

| | | |
|---|---|---|
| CDR1 | Kabat | SEQ ID NO: 1 |
| | Chothia | SEQ ID NO: 4 |
| CDR2 | Kabat | SEQ ID NO: 2 |
| | Chothia | SEQ ID NO: 5 |
| CDR3 | Kabat | SEQ ID NO: 3 |
| | Chothia | SEQ ID NO: 6 |
| VH | | SEQ ID NO: 7 |
| Heavy Chain | | SEQ ID NO: 9 |

mAb1 light chain

| | | |
|---|---|---|
| CDR1 | Kabat | SEQ ID NO: 11 |
| | Chothia | SEQ ID NO: 14 |
| CDR2 | Kabat | SEQ ID NO: 12 |
| | Chothia | SEQ ID NO: 15 |
| CDR3 | Kabat | SEQ ID NO: 13 |
| | Chothia | SEQ ID NO: 16 |
| VL | | SEQ ID NO: 17 |
| Light Chain | | SEQ ID NO: 19 |

In one embodiment, the IL-18 antibody or antigen-binding fragment thereof comprises at least one immunoglobulin heavy chain variable domain (VH) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-18 antibody or antigen-binding fragment thereof comprises at least one immunoglobulin heavy chain variable domain (VA) comprising hypervariable regions CDR1, CDR2 and CDR3, said CDR1 having the amino acid sequence SEQ ID NO:4, said CDR2 having the amino acid sequence SEQ ID NO:5, and said CDR3 having the amino acid sequence SEQ ID NO:6.

In one embodiment, the IL-18 antibody or antigen-binding fragment thereof comprises at least one immunoglobulin light chain variable domain (VL) comprising hypervariable regions CDR1, CDR2 and CDR3, said CDR1 having the amino acid sequence SEQ ID NO:11, said CDR2 having the amino acid sequence SEQ ID NO: 12 and said CDR3 having the amino acid sequence SEQ ID NO: 13. In one embodiment, the IL-18 antibody or antigen-binding fragment thereof comprises at least one immunoglobulin light chain variable domain (VL) comprising hypervariable regions CDR1, CDR2 and CDR3, said CDR1 having the amino acid sequence SEQ ID NO: 14, said CDR2 having the amino acid sequence SEQ ID NO: 15 and said CDR3 having the amino acid sequence SEQ ID NO: 16.

In one embodiment, the IL-18 antibody or antigen-binding fragment thereof comprises at least one immunoglobulin VH domain and at least one immunoglobulin VL domain, wherein: a) the immunoglobulin VH domain comprises (e.g. in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; or ii) hypervariable regions CDR1, CDR2 and CDR3, said CDR1 having the amino acid sequence SEQ ID NO:4, said CDR2 having the amino acid sequence SEQ ID NO:5, and said CDR3 having the amino acid sequence SEQ ID NO:6; and b) the immunoglobulin VL domain comprises (e.g. in sequence): i) hypervariable regions CDR1, CDR2 and CDR3, said CDR1 having the amino acid sequence SEQ ID NO:11, said CDR2 having the amino acid sequence SEQ ID NO: 12, and said CDR3 having the amino acid sequence SEQ ID NO: 13 or ii) hypervariable regions CDR1, CDR2 and CDR3, said CDR1 having the amino acid sequence SEQ ID NO: 14, said CDR2 having the amino acid sequence SEQ ID NO: 15, and said CDR3 having the amino acid sequence SEQ ID NO: 16.

In one embodiment, the IL-18 antibody or antigen-binding fragment thereof comprises: a) an immunoglobulin heavy chain variable domain (V_{H}) comprising the amino acid sequence set forth as SEQ ID NO:7; b) an immunoglobulin light chain variable domain (V_{L}) comprising the amino acid sequence set forth as SEQ ID NO: 17; c) an immunoglobulin V_{H} domain comprising the amino acid sequence set forth as SEQ ID NO:7 and an immunoglobulin V_{L} domain comprising the amino acid sequence set forth as SEQ ID NO:17; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO: 11, SEQ ID NO: 12 and SEQ ID NO: 13; f) an immunoglobulin V_{H} domain comprising the hypervariable regions set forth as SEQ ID NO:4, SEQ ID NO:5 and SEQ ID NO:6; g) an immunoglobulin V_{L} domain comprising the hypervariable regions set forth as SEQ ID NO: 14, SEQ ID NO: 15 and SEQ ID NO: 16; h) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO: 11, SEQ ID NO: 12 and SEQ ID NO:13; i) an immunoglobulin V_{H} domain comprising the hypervariable regions set forth as SEQ ID NO:4, SEQ ID NO:5, and SEQ ID NO:6 and an immunoglobulin V_{L} domain comprising the hypervariable regions set forth as SEQ ID NO: 14, SEQ ID NO: 15 and SEQ ID NO: 16; j) a light chain comprising SEQ ID NO:19; k) a heavy chain comprising SEQ ID NO: 9; or l) a light chain comprising SEQ ID NO:19 and a heavy chain comprising SEQ ID NO:9.

In some embodiments, the IL-18 antibody or antigen-binding fragment thereof (e.g. mAb1) comprises the three CDRs of SEQ ID NO:7. In other embodiments, the IL-18 antibody or antigen-binding fragment thereof comprises the three CDRs of SEQ ID NO: 17. In other embodiments, the IL-18 antibody or antigen-binding fragment thereof comprises the three CDRs of SEQ ID NO:7 and the three CDRs of SEQ ID NO:17. In some embodiments, the IL-18 antibody or antigen-binding fragment thereof comprises the three CDRs of SEQ ID NO:9. In other embodiments, IL-18 antibody or antigen-binding fragment thereof comprises the three CDRs of SEQ ID NO:19. In other embodiments, the IL-18 antibody or antigen-binding fragment thereof comprises the three CDRs of SEQ ID NO:9 and the three CDRs of SEQ ID NO: 19.

In one embodiment, the IL-18 antibody or antigen-binding fragment thereof (e.g. mAb1) is selected from a human IL-18 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO: 11, said CDR2 having the amino acid sequence SEQ ID NO: 12, and said CDR3 having the amino acid sequence SEQ ID NO: 13.

In one embodiment, the IL-18 antibody or antigen-binding fragment thereof (e.g. mAb1) is selected from a human IL-18 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:4, said CDR2 having the amino acid sequence SEQ ID NO:5 and said CDR3 having the amino acid sequence SEQ ID NO:6; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO:14, said CDR2 having the amino acid sequence SEQ ID NO: 15, and said CDR3 having the amino acid sequence SEQ ID NO: 16.

In one embodiment, the IL-18 antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1, CDR2 and CDR3, said CDR1 having the amino acid sequence SEQ ID NO: 11, said CDR2 having the amino acid sequence SEQ ID NO: 12, and said CDR3 having the amino acid sequence SEQ ID NO: 13; and c) a peptide linker which is bound either to the N-terminal extremity of the first domain and to the C-terminal extremity of the second domain or to the C-terminal extremity of the first domain and to the N-terminal extremity of the second domain.

In one embodiment, the IL-18 antibody or antigen-binding fragment thereof (e.g. mAb1) is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:4, said CDR2 having the amino acid sequence SEQ ID NO:5, and said CDR3 having the amino acid sequence SEQ ID NO:6; and b) a second domain comprising, in sequence, the hypervariable regions CDR1, CDR2 and CDR3, said CDR1 having the amino acid sequence SEQ ID NO: 14, said CDR2 having the amino acid sequence SEQ ID NO: 15, and said CDR3 having the amino acid sequence SEQ ID NO: 16; and c) a peptide linker which is bound either to the N-terminal extremity of the first domain and to the C-terminal extremity of the second domain or to the C-terminal extremity of the first domain and to the N-terminal extremity of the second domain.

The V_{H} or V_{L} domain of an IL-18 antibody or antigen-binding fragment thereof used in the disclosed methods may have V_{H} and/or V_{L} domains that are substantially identical to the V_{H} or V_{L} domains set forth in SEQ ID NO:7 and 17. A human IL-18 antibody disclosed herein may comprise a heavy chain that is substantially identical to that set forth as SEQ ID NO: 9 and/or a light chain that is substantially identical to that set forth as SEQ ID NO: 19. A human IL-18 antibody disclosed herein may comprise a heavy chain that comprises SEQ ID NO:9 and a light chain that comprises SEQ ID NO: 19. A human IL-18 antibody disclosed herein may comprise: a) one heavy chain, comprising a variable domain having an amino acid sequence substantially identical to that shown in SEQ ID NO:7 and the constant part of a human heavy chain; and b) one light chain, comprising a variable domain having an amino acid sequence substantially identical to that shown in SEQ ID NO: 17 and the constant part of a human light chain.

Other preferred IL-18 antagonists (e.g. antibodies) for use in the disclosed methods, kits and regimens are those set forth in U.S. Pat. No. 9,376,489, which is incorporated by reference herein in its entirety.

### 3. IL-1β Antibody

Particularly preferred IL-1β antibodies or antigen-binding fragments thereof used in the disclosed methods are human antibodies.

For ease of reference, the amino acid sequences of the hypervariable regions of a specific IL-1β antibody, called mAb2, based on the Kabat definition and the Chothia definition, as well as the V_{L} and V_{H} domains and full heavy and light chains are provided in Table 2, below.

TABLE 2
Amino acid sequences of the hypervariable regions (CDRs), variable
domains (VH and VL) and full chains of mAb2. The DNA encoding
the VL of mAb2 is set forth in SEQ ID NO: 38. The DNA encoding
the VH of mAb2 is set forth in SEQ ID NO: 27.
mAb2 heavy chain

| | | |
|---|---|---|
| CDR1 | Kabat | SEQ ID NO: 21 |
| | Chothia | SEQ ID NO: 24 |
| CDR2 | Kabat | SEQ ID NO: 22 |
| | Chothia | SEQ ID NO: 25 |
| CDR3 | Kabat | SEQ ID NO: 23 |
| | Chothia | SEQ ID NO: 26 |
| VH | | SEQ ID NO: 27 |
| Heavy Chain | | SEQ ID NO: 29 |

mAb2 light chain

| | | |
|---|---|---|
| CDR1 | Kabat | SEQ ID NO: 31 |
| | Chothia | SEQ ID NO: 34 |
| CDR2 | Kabat | SEQ ID NO: 32 |
| | Chothia | SEQ ID NO: 35 |
| CDR3 | Kabat | SEQ ID NO: 33 |
| | Chothia | SEQ ID NO: 36 |
| VL | | SEQ ID NO: 37 |
| Light Chain | | SEQ ID NO: 39 |

In one embodiment, the IL-13 antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO:21, said CDR2 having the amino acid sequence SEQ ID NO:22, and said CDR3 having the amino acid sequence SEQ ID NO:23. In one embodiment, the IL-1β antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO:24, said CDR2 having the amino acid sequence SEQ ID NO:25, and said CDR3 having the amino acid sequence SEQ ID NO:26.

In one embodiment, the IL-1β antibody or antigen-binding fragment thereof comprises at least one immunoglobulin light chain variable domain (V_{L}) comprising hypervariable regions CDR1, CDR2 and CDR3, said CDR1 having the amino acid sequence SEQ ID NO:31, said CDR2 having the amino acid sequence SEQ ID NO:32 and said CDR3 having the amino acid sequence SEQ ID NO:33. In one embodiment, the IL-1β antibody or antigen-binding fragment thereof comprises at least one immunoglobulin light chain variable domain (V_{L}) comprising hypervariable regions CDR1, CDR2 and CDR3, said CDR1 having the amino acid sequence SEQ ID NO:34, said CDR2 having the amino acid sequence SEQ ID NO:35 and said CDR3 having the amino acid sequence SEQ ID NO:36.

In one embodiment, the IL-1β antibody or antigen-binding fragment thereof comprises at least one immunoglobulin V_{H} domain and at least one immunoglobulin V_{L} domain, wherein: a) the immunoglobulin V_{H} domain comprises (e.g. in sequence): i) hypervariable regions CDR1, CDR2 and CDR3, said CDR1 having the amino acid sequence SEQ ID NO:21, said CDR2 having the amino acid sequence SEQ ID NO:22, and said CDR3 having the amino acid sequence SEQ ID NO:23; or ii) hypervariable regions CDR1, CDR2 and CDR3, said CDR1 having the amino acid sequence SEQ ID NO:24, said CDR2 having the amino acid sequence SEQ ID NO:25, and said CDR3 having the amino acid sequence SEQ ID NO:26; and b) the immunoglobulin V_{L} domain comprises (e.g. in sequence): i) hypervariable regions CDR1, CDR2 and CDR3, said CDR1 having the amino acid sequence SEQ ID NO:31, said CDR2 having the amino acid sequence SEQ ID NO:32, and said CDR3 having the amino acid sequence SEQ ID NO:33 or ii) hypervariable regions CDR1, CDR2 and CDR3, said CDR1 having the amino acid sequence SEQ ID NO:34, said CDR2 having the amino acid sequence SEQ ID NO:35, and said CDR3 having the amino acid sequence SEQ ID NO:36.

In one embodiment, the IL-1β antibody or antigen-binding fragment thereof comprises: a) an immunoglobulin heavy chain variable domain (V_{H}) comprising the amino acid sequence set forth as SEQ ID NO:27; b) an immunoglobulin light chain variable domain (V_{L}) comprising the amino acid sequence set forth as SEQ ID NO:37; c) an immunoglobulin V_{H} domain comprising the amino acid sequence set forth as SEQ ID NO:27 and an immunoglobulin V_{L} domain comprising the amino acid sequence set forth as SEQ ID NO:37; d) an immunoglobulin V_{H} domain comprising the hypervariable regions set forth as SEQ ID NO:21, SEQ ID NO:22, and SEQ ID NO:23; e) an immunoglobulin V_{L} domain comprising the hypervariable regions set forth as SEQ ID NO:31, SEQ ID NO:32 and SEQ ID NO:33; f) an immunoglobulin V_{H} domain comprising the hypervariable regions set forth as SEQ ID NO:24, SEQ ID NO:25 and SEQ ID NO:26; g) an immunoglobulin V_{L} domain comprising the hypervariable regions set forth as SEQ ID NO:34, SEQ ID NO:35 and SEQ ID NO:36; h) an immunoglobulin V_{H} domain comprising the hypervariable regions set forth as SEQ ID NO:21, SEQ ID NO:22, and SEQ ID NO:23 and an immunoglobulin V_{L} domain comprising the hypervariable regions set forth as SEQ ID NO:31, SEQ ID NO:32 and SEQ ID NO:33; i) an immunoglobulin V_{H} domain comprising the hypervariable regions set forth as SEQ ID NO:24, SEQ ID NO: 25, and SEQ ID NO:26 and an immunoglobulin V_{L} domain comprising the hypervariable regions set forth as SEQ ID NO:34, SEQ ID NO:35 and SEQ ID NO:36; j) a light chain comprising SEQ ID NO:37; k) a heavy chain comprising SEQ ID NO:29; or l) a light chain comprising SEQ ID NO:39 and a heavy chain comprising SEQ ID NO:29.

In some embodiments, the IL-1β antibody or antigen-binding fragment thereof (e.g. mAb2) comprises the three CDRs of SEQ ID NO:37. In other embodiments, the IL-1β antibody or antigen-binding fragment thereof comprises the three CDRs of SEQ ID NO:27. In other embodiments, the IL-1β antibody or antigen-binding fragment thereof comprises the three CDRs of SEQ ID NO:37 and the three CDRs of SEQ ID NO:27. In some embodiments, the IL-1β antibody or antigen-binding fragment thereof comprises the three CDRs of SEQ ID NO:39. In other embodiments, IL-1β antibody or antigen-binding fragment thereof comprises the three CDRs of SEQ ID NO:29. In other embodiments, the IL-1β antibody or antigen-binding fragment thereof comprises the three CDRs of SEQ ID NO:39 and the three CDRs of SEQ ID NO:29.

In one embodiment, the IL-1β antibody or antigen-binding fragment thereof (e.g. mAb2) is selected from a human IL-1β antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:21, said CDR2 having the amino acid sequence SEQ ID NO:22, and said CDR3 having the amino acid sequence SEQ ID NO:23; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO:31, said CDR2 having the amino acid sequence SEQ ID NO:32, and said CDR3 having the amino acid sequence SEQ ID NO:33.

In one embodiment, the IL-1β antibody or antigen-binding fragment thereof (e.g. mAb2) is selected from a human IL-1β antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:24, said CDR2 having the amino acid sequence SEQ ID NO:25 and said CDR3 having the amino acid sequence SEQ ID NO:26; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO:34, said CDR2 having the amino acid sequence SEQ ID NO:35, and said CDR3 having the amino acid sequence SEQ ID NO:36.

In one embodiment, the IL-1β antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:21, said CDR2 having the amino acid sequence SEQ ID NO:22, and said CDR3 having the amino acid sequence SEQ ID NO:23; and b) a second domain comprising, in sequence, the hypervariable regions CDR1, CDR2 and CDR3, said CDR1 having the amino acid sequence SEQ ID NO:31, said CDR2 having the amino acid sequence SEQ ID NO:32, and said CDR3 having the amino acid sequence SEQ ID NO:33; and c) a peptide linker which is bound either to the N-terminal extremity of the first domain and to the C-terminal extremity of the second domain or to the C-terminal extremity of the first domain and to the N-terminal extremity of the second domain.

In one embodiment, the IL-1β antibody or antigen-binding fragment thereof (e.g. mAb2) is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:24, said CDR2 having the amino acid sequence SEQ ID NO:25, and said CDR3 having the amino acid sequence SEQ ID NO:26; and b) a second domain comprising, in sequence, the hypervariable regions CDR1, CDR2 and CDR3, said CDR1 having the amino acid sequence SEQ ID NO:34, said CDR2 having the amino acid sequence SEQ ID NO:35, and said CDR3 having the amino acid sequence SEQ ID NO:36; and c) a peptide linker which is bound either to the N-terminal extremity of the first domain and to the C-terminal extremity of the second domain or to the C-terminal extremity of the first domain and to the N-terminal extremity of the second domain.

The V_{H} or V_{L} domain of an IL-1β antibody or antigen-binding fragment thereof used in the disclosed methods may have V_{H} and/or V_{L} domains that are substantially identical to the V_{H} or V_{L} domains set forth in SEQ ID NO:27 and 37. A human IL-1β antibody disclosed herein may comprise a heavy chain that is substantially identical to that set forth as SEQ ID NO:29 and/or a light chain that is substantially identical to that set forth as SEQ ID NO:39. A human IL-1β antibody disclosed herein may comprise a heavy chain that comprises SEQ ID NO:29 and a light chain that comprises SEQ ID NO:39. A human IL-1β antibody disclosed herein may comprise: a) one heavy chain, comprising a variable domain having an amino acid sequence substantially identical to that shown in SEQ ID NO:27 and the constant part of a human heavy chain; and b) one light chain, comprising a variable domain having an amino acid sequence substantially identical to that shown in SEQ ID NO:37 and the constant part of a human light chain.

Other preferred IL-1β antagonists (e.g. antibodies) for use in the disclosed methods, kits and regimens are those set forth in US Pat. Nos: 7,446, 175 or 7,993,878 or 8,273,350, which are incorporated by reference herein in their entirety.

### 4. Fc Modifications

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g. one or more chemical moieties can be attached to the antibody) or be modified to alter its glycosylation, again to alter one or more functional properties of the antibody. Each of these embodiments is described in further detail below. The numbering of residues in the Fc region is that of the EU numbering scheme of Edelman et al., PNAS, 1969 May, 63(1): 78-85.

In one embodiment, the hinge region of CH1 is modified such that the number of cysteine residues in the hinge region is altered, e.g. increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al, (2001) J Biol Chem 276:6591-6604).

In certain embodiments, the Fc domain of IgG1 isotype is used. In some specific embodiments, a mutant variant of IgG1 Fc fragment is used, e.g. a silent IgG1 Fc which reduces or eliminates the ability of the fusion polypeptide to mediate antibody dependent cellular cytotoxicity (ADCC) and/or to bind to an Fcγ receptor. An example of an IgG1 isotype silent mutant wherein Leucine residue is replaced by Alanine residue at amino acid positions 234 and 235 as described by Hezareh et al, J. Virol (2001); 75(24): 12161-8.

In certain embodiments, the Fc domain is a mutant preventing glycosylation at position 297 of Fc domain. For example, the Fc domain contains an amino acid substitution of asparagine residue at position 297. Example of such amino acid substitution is the replacement of N297 by a glycine or an alanine.

Silenced effector functions can be obtained by mutation in the Fc region of the antibodies and have been described in the art: LALA and N297A (Strohl, W., 2009, Curr. Opin. Biotechnol. vol. 20(6):685-691); and D265A (Baudino et al., 2008, J. Immunol. 181:6664-69; Strohl, W., supra); and DAPA (D265A and P329A) (Shields R L., J Biol Chem. 2001; 276(9):6591-604; U.S. Patent Publication US2015/0320880). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another example of a silent IgG1 antibody is the so-called DAPA mutant, comprising D265A and P329A mutations to the IgG1 Fc amino acid sequence. Another silent IgG1 antibody comprises the N297A mutation, which results in aglycosylated/non-glycosylated antibodies. Additional Fc mutations for providing silenced effector function are described in PCT publication no. WO2014/145806 (e.g., in FIG. 7 of WO2014/145806), herein incorporated by reference in its entirety. One example from WO2014/145806 of a silent IgG1 antibody comprises a E233P, L234V, L235A, and S267K mutation, and a deletion of G236 (G236del). Another example from WO2014/145806 of a silent IgG1 antibody comprises a E233P, L234V, and L235A mutation, and a deletion of G236 (G236del). Another example from WO2014/145806 of a silent IgG1 antibody comprises a S267K mutation.

In still another embodiment, the glycosylation of an antibody is modified. For example, an aglycosylated antibody can be made (i.e., the antibody lacks glycosylation). Glycosylation can be altered to, for example, increase the affinity of the antibody for the antigen. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176, 195 by Hang et al. describes a cell line with a functionally disrupted FUT8 gene, which encodes a fucosyl transferase, such that antibodies expressed in such a cell line exhibit hypofucosylation. Therefore, in one embodiment, the antibodies of the invention are produced by recombinant expression in a cell line which exhibit hypofucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g. beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). Alternatively, the antibodies of the invention can be produced in a yeast or a filamentous fungi engineered for mammalian-like glycosylation pattern, and capable of producing antibodies lacking fucose as glycosylation pattern (see for example EP1297172B1).

Another modification of the antibodies herein that is contemplated by the invention is pegylation. An antibody can be pegylated to, for example, increase the biological (e.g. serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

Another modification of the antibodies that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094.

Another modification of the antibodies that is contemplated by the invention is one or more modifications to increase formation of a heterodimeric bispecific antibody. A variety of approaches available in the art can be used in for enhancing dimerization of the two heavy chain domains of bispecific antibodies, e.g., bbmAbs, as disclosed in, for example, EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1, the contents of which are incorporated herein in their entireties.

Generation of bispecific antibodies using knobs-into-holes is disclosed e.g. in PCT Publication No. WO1996/027011, Ridgway et al., (1996), and Merchant et al. (1998).

In practicing some of the methods of treatment or uses of the present disclosure, a therapeutically effective amount of a bispecific antibodies targeting both IL-1β and IL-18 simultaneously, e.g. bbmAb1 has to be administered to a subject in need thereof. It will be understood that regimen changes may be appropriate for certain patients. Thus, administration (e.g. of bbmAb1) may be more frequent e.g., daily, bi-weekly dosing, or weekly dosing.

Some patients may benefit from a loading regimen (e.g., daily administrations for several days/[e.g., 1 to 4 days e.g., dosing at day 0, 1, 2, and/or 3] followed by a maintenance regimen starting e.g. at Week 3 or 4 where bbmAb1 may be administered weekly, bi-weekly or every 4 weeks for several weeks. In some embodiments, the period of administration of a a bispecific antibodies targeting both IL-1β and IL-18 simultaneously, e.g. bbmAb1 is for 1 day, 2 days, 3 days, 4 days, 5 days, 6 days, 7 days. In some embodiments, the period of administration of a a bispecific antibodies targeting both IL-1β and IL-18 simultaneously, e.g. bbmAb1 is for 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 1 1 months, 12 months, or more.

It will be understood that dose escalation may be appropriate for certain patients, for example patients, based on severity of the disease, e.g., patients that display inadequate response to treatment with the bbmAb1. Thus, dosages (intravenous (i.v.)) may be greater than about 10 mg/kg, e.g., about 11 mg/kg, 12 mg/kg, 15 mg/kg, 20 mg/kg, 25 mg/kg, 30 mg/kg, 35 mg/kg, etc. Furthermore, subcutaneous (s.c.) dosages (loading or maintenance doses) may be greater than about 50 mg to about 900 mg s.c., e.g., about 75 mg, about 100 mg, about 125 mg, about 175 mg, about 200 mg, about 250 mg, about 350 mg, about 400 mg, about 450 mg, about 500 mg, about 600 mg, etc.;

It will also be understood that dose reduction may also be appropriate for certain patients, such as patients, e.g., patients that display adverse events or an adverse response to treatment with the bbmAb1. Thus, dosages of the may be less than about 10 mg/kg e.g., about 1 mg/kg, about 2 mg/kg, about 3 mg/kg, about 4 mg/kg, about 5 mg/kg, about 6 mg/kg, about 7 mg/kg, about 8 mg/kg or about 9 mg/kg. In some embodiments, the bbmAB1 dose may be adjusted as determined by a physician.

In some embodiments, the bbmAB1 antibody may be administered to the patient as a single dose of 10 mg/kg delivered i.v., wherein the dose may be adjusted to a higher or lower dose if needed, as determined by a physician, e.g., about 1 mg/kg, about 2 mg/kg, about 3 mg/kg, about 4 mg/kg, about 5 mg/kg, about 6 mg/kg, about 7 mg/kg, about 8 mg/kg or about 9 mg/kg or e.g., about 11 mg/kg, 12 mg/kg, 15 mg/kg, 20 mg/kg, 25 mg/kg, 30 mg/kg, 35 mg/kg, etc.

In some embodiments, the bbmAB1 antibody may be administered to the patient at an initial dose of 10 mg/kg delivered i.v., and the dose may be then adjusted to a higher or lower dose if needed, as determined by a physician.

In a specific embodiment, 10 mg/kg bbmAB1 is administered on day 1.

In a specific embodiment, 10 mg/kg bbmAB1 is administered on day 1 (D1) and on day 2 (D2), D3, D4, D5, D6, D7, D8, D9, D10, D11, D12, D13 and/or D14

In another specific embodiment, 10 mg/kg bbmAB1 is administered i.v. on day 1.

### Example 1: Multiple Inflammasomes are Likely to be Involved in HS

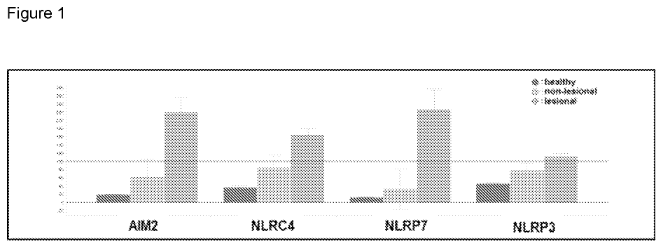

Analysis of the HS transcriptome from patients show an increase of mRNA levels of the AIM2, NLRC4, NLRP7 and NLRP3 inflammasomes in HS lesions compared to levels of non-lesional or healthy tissue (FIG. 1). The expression levels of all these inflammasome genes is clearly higher in HS lesional samples than in HS non-lesional samples and healthy samples, which indicates that multiple inflammasomes are likely to be involved in HS pathophysiology.

### Example 2: Both IL-1β and IL-18 Signaling Pathways are Active in HS

### (a) Transcriptomics of Skin Biopsies

Transcriptomics analysis of 18 lesional HS biopsies, 6 peri-lesional and 7 non-lesional biopsies versus 8 biopsies from healthy skin donors were derived as follows: From snap frozen skin tissue, a homogenate was prepared using recommended buffers from Qiagen RNeasy mini kit. The total RNA of the cells was extracted according to manufacturer's protocol. cDNA of the samples was prepared from the same starting amount of RNA using a High capacity cDNA reverse Transcription Kit (Applied Biosystems). Samples were processed by CiToxLAB France on Affymetrix HG_U133_Plus2 microarrays. RMA normalized data was analyzed using GeneSpring 11.5.1 (Agilent Technologies, Santa Clara, CA). Initially, the data was subject to standard QC control by CiToxLAB and in GeneSpring (PCA, hybridization controls). Subsequently, it was filtered on expression levels to probesets above the 20th percentile in 100% of the samples in any one of the conditions before further analysis. The dataset is deposited in NCBI GEO database (GSE148027). Results were visualized using TIBCO Sporfire Analyst.

(b) Transcriptomics of Cytokine Stimulated PBMC (from Healthy Donors)

Stimulation of PBMC with recombinant cytokines was performed by performed with 7×106 PBMC per well of a 12-well plate in 1.5 ml final volume in RPMI medium. Recombinant cytokines were added at the following final concentrations: 10 ng/ml of recombinant IL-1β, 3 nM of recombinant IL-18 and 1 ng/ml of recombinant IL-12. Cells were collected after 6 hours of stimulation in cell culture at 370C and 5% CO2.

For RNA isolation, cells were pelleted and the pellet lysed in 350 μl of Qiagen RTL buffer with 2% β-mercaptoethanol and frozen at −20° C. or −80° C. until all samples of the study have been collected. The RNA isolation was performed using the Qiagen standard protocol. The different RNA samples were processed by CiToxLAB France on Affymetrix HG_U133_Plus2 microarrays as described above. Entities (probesets) were kept where at least 100 percent of samples in any 1 of the experimental conditions have values above the 20th percentile. Differentially expressed genes (DEG) were identified using the “filter on volcano plot” feature in GeneSpring Using the filtered genes (expression between 20.0-100.0th percentiles) with an unpaired T-test, probesets with a corrected p-value below 0.05 and a fold change above 2.0 were considered differentially expressed. Where possible, a Benjamini-Hochberg Multiple Testing Correction was used.

The resulting gene lists were used as “cytokine signaling signatures” and the expression levels of the defined signature was interrogated in the HS transcriptomic dataset without using any associated values from the original experiment.

### (c) Generation of the Spotfire Transcriptomic Heat Map

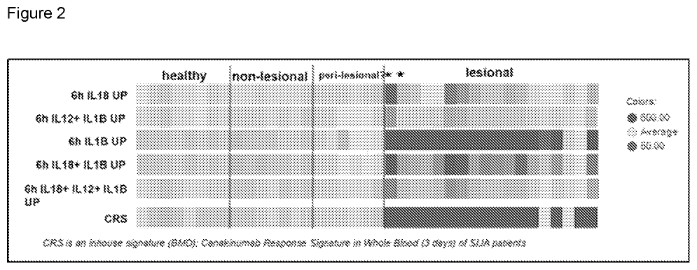

The expression levels of the differentially upregulated genes obtained from the cytokine stimulated PBMC were averaged for every single skin biopsy and a color coding with increasing color intensity was attributed to the increased average expression levels of the respective PBMC signature (FIG. 2).

Providing evidence that both IL-1b as well as IL-18 signaling is present and active in lesional skin of HS patients and that these two cytokines are likely to play a role in HS pathophysiology.

### Example 3

The generation of bbmAb1 has been described in detail in the examples 1 to 5 of the patent application WO/2018/229612. The examples 1 of WO/2018/229612, comprising (1) vector construction, (2) Host cell line and transfection, (3) Cell selection and sorting, (4) Cell expansion, (5) Clone stability, (6) Manufacturing, (7) Analytical characterization and purity assessment, (8) Analytical Results are herewith incorporated by reference in their entirety.

The bbmAb1, is a bispecific IgG1, with LALA silencing mutations, simultaneously binding to two distinct targets, IL-1β and IL-18. The antibody combines two distinct antigen binding arms (Fab fragments), whereas the Fab directed against IL-1β is based on mAb2 and contains a kappa light chain (Vk6). The Fab directed against IL-18 is based on mAb1 and is composed of a lambda light chain (Vλ1). In order to drive hetero-dimerization of the Fc domain during expression a “knob” with a bulky amino acid (aa) side chain (S354C and T366W) in the mAb1 heavy chain and a “hole” with small aa side chains (Y349C, T366S, L368A, Y407V) were introduced in the mAb2 heavy chain.

For ease of reference, the amino acid sequences of the hypervariable regions of bbmAb1, based on the Kabat definition and the Chothia definition, as well as the V_{L} and V_{H} domains and full heavy and light chains are provided in Table 3 below.

TABLE 3
Amino acid sequences of the hypervariable regions (CDRs), variable
domains (VH and VL) and full chains of bbmAb1. The DNA encoding
the first VL of is set forth in SEQ ID NO: 102 and the DNA
encoding the second VL is set forth in SEQ ID NO: 70. The DNA
encoding the first VH is set forth in SEQ ID NO: 86 and the
DNA encoding the second VH is set forth in SEQ ID NO: 54.
bbmAb1 heavy chain 1 (from mAb1)

| | | |
|---|---|---|
| CDR1-1 | Kabat | SEQ ID NO: 76 |
| | Chothia | SEQ ID NO: 79 |
| | IMGT | SEQ ID NO: 82 |
| CDR2-1 | Kabat | SEQ ID NO: 77 |
| | Chothia | SEQ ID NO: 80 |
| | IMGT | SEQ ID NO: 83 |
| CDR3-1 | Kabat | SEQ ID NO: 78 |
| | Chothia | SEQ ID NO: 81 |
| | IMGT | SEQ ID NO: 84 |
| VH-1 | | SEQ ID NO: 85 |
| Heavy Chain-1 | | SEQ ID NO: 87 |

bbmAb1 light chain 1 (from mAb1)

| | | |
|---|---|---|
| CDR1-1 | Kabat | SEQ ID NO: 92 |
| | Chothia | SEQ ID NO: 95 |
| | IMGT | SEQ ID NO: 98 |
| CDR2-1 | Kabat | SEQ ID NO: 93 |
| | Chothia | SEQ ID NO: 96 |
| | IMGT | SEQ ID NO: 99 |
| CDR3-1 | Kabat | SEQ ID NO: 94 |
| | Chothia | SEQ ID NO: 97 |
| | IMGT | SEQ ID NO: 100 |
| VL-1 | | SEQ ID NO: 101 |
| Light Chain-1 | | SEQ ID NO: 103 |

bbmAb1 heavy chain 2 (from mAb2)

| | | |
|---|---|---|
| CDR1-2 | Kabat | SEQ ID NO: 44 |
| | Chothia | SEQ ID NO: 47 |
| | IMGT | SEQ ID NO: 50 |
| CDR2-2 | Kabat | SEQ ID NO: 45 |
| | Chothia | SEQ ID NO: 48 |
| | IMGT | SEQ ID NO: 51 |
| CDR3-2 | Kabat | SEQ ID NO: 46 |
| | Chothia | SEQ ID NO: 49 |
| | IMGT | SEQ ID NO: 52 |
| VH-2 | | SEQ ID NO: 53 |
| Heavy Chain-2 | | SEQ ID NO: 55 |

bbmAb1 light chain 2 (from mAb2)

| | | |
|---|---|---|
| CDR1-2 | Kabat | SEQ ID NO: 60 |
| | Chothia | SEQ ID NO: 63 |
| | IMGT | SEQ ID NO: 66 |
| CDR2-2 | Kabat | SEQ ID NO: 61 |
| | Chothia | SEQ ID NO: 64 |
| | IMGT | SEQ ID NO: 67 |
| CDR3-2 | Kabat | SEQ ID NO: 62 |
| | Chothia | SEQ ID NO: 65 |
| | IMGT | SEQ ID NO: 68 |
| VL-2 | | SEQ ID NO: 69 |
| Light Chain-2 | | SEQ ID NO: 71 |

In one embodiment, the IL-18/IL-1β bispecific antibody for use in the treatment or prevention of HS, comprises a first immunoglobulin heavy chain variable domain (V_{H1}) comprising hypervariable regions CDR1, CDR2 and CDR3, said CDR1 having the amino acid sequence SEQ ID NO:76, said CDR2 having the amino acid sequence SEQ ID NO:77, and said CDR3 having the amino acid sequence SEQ ID NO:78. In one embodiment, IL-18/IL-1β bispecific antibody for use in the treatment or prevention of HS, comprises a first immunoglobulin heavy chain variable domain (V_{H1}) comprising hypervariable regions CDR1, CDR2 and CDR3, said CDR1 having the amino acid sequence SEQ ID NO:79, said CDR2 having the amino acid sequence SEQ ID NO:80, and said CDR3 having the amino acid sequence SEQ ID NO:81. In one embodiment, IL-18/IL-1β bispecific antibody for use in (i) the disclosed treatment or prevention of HS, comprises a first immunoglobulin heavy chain variable domain (V_{H1}) comprising hypervariable regions CDR1, CDR2 and CDR3, said CDR1 having the amino acid sequence SEQ ID NO:82, said CDR2 having the amino acid sequence SEQ ID NO:83, and said CDR3 having the amino acid sequence SEQ ID NO:84.

In one embodiment, the IL-18/IL-1β bispecific antibody for use in treatment or prevention of HS, comprises a second immunoglobulin heavy chain variable domain (V_{H2}) comprising hypervariable regions CDR1, CDR2 and CDR3, said CDR1 having the amino acid sequence SEQ ID NO:44, said CDR2 having the amino acid sequence SEQ ID NO:45, and said CDR3 having the amino acid sequence SEQ ID NO:46. In one embodiment, the IL-18/IL-1β bispecific antibody for use in treatment or prevention of HS, comprises a second immunoglobulin heavy chain variable domain (V_{H2}) comprising hypervariable regions CDR1, CDR2 and CDR3, said CDR1 having the amino acid sequence SEQ ID NO:47, said CDR2 having the amino acid sequence SEQ ID NO:48, and said CDR3 having the amino acid sequence SEQ ID NO:49. In one embodiment, the IL-18/IL-1β bispecific antibody for use in treatment or prevention of HS, comprises a second immunoglobulin heavy chain variable domain (V_{H2}) comprising hypervariable regions CDR1, CDR2 and CDR3, said CDR1 having the amino acid sequence SEQ ID NO:50, said CDR2 having the amino acid sequence SEQ ID NO:51, and said CDR3 having the amino acid sequence SEQ ID NO:52.

In one embodiment, the IL-18/IL-1β bispecific antibody for use in treatment or prevention of HS, comprises a first immunoglobulin light chain variable domain (V_{L1}) comprising hypervariable regions CDR1, CDR2 and CDR3, said CDR1 having the amino acid sequence SEQ ID NO:92, said CDR2 having the amino acid sequence SEQ ID NO:93 and said CDR3 having the amino acid sequence SEQ ID NO:94. In one embodiment, the IL-18/IL-1β bispecific antibody for use in treatment or prevention of HS, comprises a first immunoglobulin light chain variable domain (V_{L1}) comprising hypervariable regions CDR1, CDR2 and CDR3, said CDR1 having the amino acid sequence SEQ ID NO:95, said CDR2 having the amino acid sequence SEQ ID NO:96 and said CDR3 having the amino acid sequence SEQ ID NO:97. In one embodiment, the IL-18/IL-1β bispecific antibody for use in treatment or prevention of HS, comprises a first immunoglobulin light chain variable domain (V_{L1}) comprising hypervariable regions CDR1, CDR2 and CDR3, said CDR1 having the amino acid sequence SEQ ID NO:98, said CDR2 having the amino acid sequence SEQ ID NO:99 and said CDR3 having the amino acid sequence SEQ ID NO:100.

In one embodiment, the IL-18/IL-1β bispecific antibody for use in treatment or prevention of HS, comprises a second immunoglobulin light chain variable domain (V_{L2}) comprising hypervariable regions CDR1, CDR2 and CDR3, said CDR1 having the amino acid sequence SEQ ID NO:60, said CDR2 having the amino acid sequence SEQ ID NO:61 and said CDR3 having the amino acid sequence SEQ ID NO:62. In one embodiment, the IL-18/IL-1β bispecific antibody for use in treatment or prevention of HS, comprises a second immunoglobulin light chain variable domain (V_{L2}) comprising hypervariable regions CDR1, CDR2 and CDR3, said CDR1 having the amino acid sequence SEQ ID NO:63, said CDR2 having the amino acid sequence SEQ ID NO:64 and said CDR3 having the amino acid sequence SEQ ID NO:65. In one embodiment, the IL-18/IL-1β bispecific antibody for use in treatment or prevention of HS, comprises a second immunoglobulin light chain variable domain (V_{L2}) comprising hypervariable regions CDR1, CDR2 and CDR3, said CDR1 having the amino acid sequence SEQ ID NO:66, said CDR2 having the amino acid sequence SEQ ID NO:67 and said CDR3 having the amino acid sequence SEQ ID NO:68.

In one embodiment, the IL-18/IL-1β bispecific antibody for use in treatment or prevention of HS, comprises a first immunoglobulin V_{H1} domain and a first immunoglobulin V_{L1} domain, wherein: a) the first immunoglobulin V_{H1} domain comprises (e.g. in sequence): i) hypervariable regions CDR1, CDR2 and CDR3, said CDR1 having the amino acid sequence SEQ ID NO:76, said CDR2 having the amino acid sequence SEQ ID NO:77, and said CDR3 having the amino acid sequence SEQ ID NO:78; or ii) hypervariable regions CDR1, CDR2 and CDR3, said CDR1 having the amino acid sequence SEQ ID NO:79, said CDR2 having the amino acid sequence SEQ ID NO:80, and said CDR3 having the amino acid sequence SEQ ID NO:81; or iii) hypervariable regions CDR1, CDR2 and CDR3, said CDR1 having the amino acid sequence SEQ ID NO:82, said CDR2 having the amino acid sequence SEQ ID NO:83, and said CDR3 having the amino acid sequence SEQ ID NO:84 and b) the first immunoglobulin V_{L1} domain comprises (e.g. in sequence): i) hypervariable regions CDR1, CDR2 and CDR3, said CDR1 having the amino acid sequence SEQ ID NO:92, said CDR2 having the amino acid sequence SEQ ID NO:93, and said CDR3 having the amino acid sequence SEQ ID NO:94 or ii) hypervariable regions CDR1, CDR2 and CDR3, said CDR1 having the amino acid sequence SEQ ID NO:95, said CDR2 having the amino acid sequence SEQ ID NO:96, and said CDR3 having the amino acid sequence SEQ ID NO:97 or iii) hypervariable regions CDR1, CDR2 and CDR3, said CDR1 having the amino acid sequence SEQ ID NO:98, said CDR2 having the amino acid sequence SEQ ID NO:99, and said CDR3 having the amino acid sequence SEQ ID NO:100.

In one embodiment, the IL-18/IL-1β bispecific antibody for use in treatment or prevention of HS, comprises a second immunoglobulin V_{H2} domain and a second immunoglobulin V_{L2} domain, wherein: a) the second immunoglobulin V_{H2} domain comprises (e.g. in sequence): i) hypervariable regions CDR1, CDR2 and CDR3, said CDR 1 having the amino acid sequence SEQ ID NO:44, said CDR2 having the amino acid sequence SEQ ID NO:45, and said CDR3 having the amino acid sequence SEQ ID NO:46; or ii) hypervariable regions CDR1, CDR2 and CDR3, said CDR1 having the amino acid sequence SEQ ID NO:47, said CDR2 having the amino acid sequence SEQ ID NO:48, and said CDR3 having the amino acid sequence SEQ ID NO:49; or iii) hypervariable regions CDR1, CDR2 and CDR3, said CDR1 having the amino acid sequence SEQ ID NO:50, said CDR2 having the amino acid sequence SEQ ID NO:51, and said CDR3 having the amino acid sequence SEQ ID NO:52 and b) the second immunoglobulin V_{L2} domain comprises (e.g. in sequence): i) hypervariable regions CDR1, CDR2 and CDR3, said CDR 1 having the amino acid sequence SEQ ID NO:60, said CDR2 having the amino acid sequence SEQ ID NO:61, and said CDR3 having the amino acid sequence SEQ ID NO:62 or ii) hypervariable regions CDR1, CDR2 and CDR3, said CDR1 having the amino acid sequence SEQ ID NO:63, said CDR2 having the amino acid sequence SEQ ID NO:64, and said CDR3 having the amino acid sequence SEQ ID NO:65 or iii) hypervariable regions CDR1, CDR2 and CDR3, said CDR1 having the amino acid sequence SEQ ID NO:66, said CDR2 having the amino acid sequence SEQ ID NO:67, and said CDR3 having the amino acid sequence SEQ ID NO:68.

In one embodiment, the IL-18/IL-1β bispecific antibody for use in treatment or prevention of HS, comprises: a) a first immunoglobulin heavy chain variable domain (V_{H1}) comprising the amino acid sequence set forth as SEQ ID NO:85; b) a first immunoglobulin light chain variable domain (V_{L1}) comprising the amino acid sequence set forth as SEQ ID NO: 101; c) a first immunoglobulin V_{H1} domain comprising the amino acid sequence set forth as SEQ ID NO:85 and a first immunoglobulin V_{L1} domain comprising the amino acid sequence set forth as SEQ ID NO: 101; d) a first immunoglobulin V_{H1} domain comprising the hypervariable regions set forth as SEQ ID NO:76, SEQ ID NO:77, and SEQ ID NO:78; e) a first immunoglobulin V_{L1} domain comprising the hypervariable regions set forth as SEQ ID NO:92, SEQ ID NO:93 and SEQ ID NO:94; f) a first immunoglobulin V_{H1} domain comprising the hypervariable regions set forth as SEQ ID NO:79, SEQ ID NO:80 and SEQ ID NO:81; g) a first immunoglobulin V_{L1} domain comprising the hypervariable regions set forth as SEQ ID NO:95, SEQ ID NO:96 and SEQ ID NO:97; h) a first immunoglobulin V_{H1} domain comprising the hypervariable regions set forth as SEQ ID NO:76, SEQ ID NO:77, and SEQ ID NO:78 and a first immunoglobulin V_{L1} domain comprising the hypervariable regions set forth as SEQ ID NO:92, SEQ ID NO:93 and SEQ ID NO:94; i) a first immunoglobulin V_{H1} domain comprising the hypervariable regions set forth as SEQ ID NO:79, SEQ ID NO:80, and SEQ ID NO:81 and a first immunoglobulin V_{L1} domain comprising the hypervariable regions set forth as SEQ ID NO:95, SEQ ID NO:96 and SEQ ID NO:97; j) a first light chain comprising SEQ ID NO: 103; k) a first heavy chain comprising SEQ ID NO: 87; or l) a first light chain comprising SEQ ID NO: 103 and a first heavy chain comprising SEQ ID NO:87.

In one embodiment, the IL-18/IL-1β bispecific antibody for use in treatment or prevention of HS, comprises: a) a second immunoglobulin heavy chain variable domain (VH2) comprising the amino acid sequence set forth as SEQ ID NO:53; b) a second immunoglobulin light chain variable domain (V_{L2}) comprising the amino acid sequence set forth as SEQ ID NO:69; c) a second immunoglobulin V_{H2} domain comprising the amino acid sequence set forth as SEQ ID NO:53 and a second immunoglobulin V_{L2} domain comprising the amino acid sequence set forth as SEQ ID NO:69; d) a second immunoglobulin V_{H2} domain comprising the hypervariable regions set forth as SEQ ID NO:44, SEQ ID NO:45, and SEQ ID NO:46; e) a second immunoglobulin V_{L2} domain comprising the hypervariable regions set forth as SEQ ID NO:60, SEQ ID NO:61 and SEQ ID NO:62; f) a second immunoglobulin V_{H2} domain comprising the hypervariable regions set forth as SEQ ID NO:47, SEQ ID NO:48 and SEQ ID NO:49; g) a second immunoglobulin V_{L2} domain comprising the hypervariable regions set forth as SEQ ID NO:63, SEQ ID NO:64 and SEQ ID NO:65; h) a second immunoglobulin V_{H2} domain comprising the hypervariable regions set forth as SEQ ID NO:44, SEQ ID NO:45, and SEQ ID NO:46 and a second immunoglobulin V_{L2} domain comprising the hypervariable regions set forth as SEQ ID NO:60, SEQ ID NO:61 and SEQ ID NO:62; i) a second immunoglobulin V_{H2} domain comprising the hypervariable regions set forth as SEQ ID NO:47, SEQ ID NO:48, and SEQ ID NO:49 and a second immunoglobulin V_{L2} domain comprising the hypervariable regions set forth as SEQ ID NO:63, SEQ ID NO:64 and SEQ ID NO:65; j) a second light chain comprising SEQ ID NO:81; k) a second heavy chain comprising SEQ ID NO:55; or l) a second light chain comprising SEQ ID NO:81 and a second heavy chain comprising SEQ ID NO:55.

In some embodiments, the IL-18/IL-1β bispecific antibody for use in treatment or prevention of HS, comprises three CDRs of SEQ ID NO:53. In other embodiments, the IL-18/IL-1β bispecific antibody for use in treatment or prevention of HS, comprises the three CDRs of SEQ ID NO:69. In other embodiments, the IL-18/IL-1β bispecific antibody for use in treatment or prevention of HS, comprises the three CDRs of SEQ ID NO:53 and the three CDRs of SEQ ID NO:69. In some embodiments, the IL-18/IL-1β bispecific antibody for use in treatment or prevention of HS, comprises the three CDRs of SEQ ID NO:85. In other embodiments, the IL-18/IL-1β bispecific antibody for use in treatment or prevention of HS, comprises the three CDRs of SEQ ID NO: 101. In other embodiments, the IL-18/IL-1β bispecific antibody for use in treatment or prevention of HS, comprises the three CDRs of SEQ ID NO:85 and the three CDRs of SEQ ID NO: 101.

In some embodiments, the IL-18/IL-1β bispecific antibody for use in treatment or prevention of HS, comprises a the three CDRs of SEQ ID NO:85. In other embodiments, the IL-18/IL-1β bispecific antibody for use in treatment or prevention of HS, comprises the three CDRs of SEQ ID NO: 101. In other embodiments, the IL-18/IL-1β bispecific antibody for use in treatment or prevention of HS, comprises the three CDRs of SEQ ID NO:85 and the three CDRs of SEQ ID NO: 101. In some embodiments, the IL-18/IL-1β bispecific antibody for use in treatment or prevention of HS, comprises the three CDRs of SEQ ID NO:53. In other embodiments, the IL-18/IL-1β bispecific antibody for use in treatment or prevention of HS, comprises the three CDRs of SEQ ID NO:69. In other embodiments, the IL-18/IL-1β bispecific antibody for use in treatment or prevention of HS, comprises the three CDRs of SEQ ID NO:53 and the three CDRs of SEQ ID NO:69. In an embodiment, the L-18/IL-1β bispecific antibody for use in treatment or prevention of HS comprises the three CDRs of SEQ ID NO: 85, the three CDRs of SEQ ID NO: 101, the three CDRs of SEQ ID NO:53 and the three CDRs of SEQ ID NO:69.

In one embodiment, the first part of the IL-18/IL-1β bispecific antibody for use in treatment or prevention of HS is selected from a human IL-18 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR 1 having the amino acid sequence SEQ ID NO:76, said CDR2 having the amino acid sequence SEQ ID NO:77, and said CDR3 having the amino acid sequence SEQ ID NO:78; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO:92, said CDR2 having the amino acid sequence SEQ ID NO:93, and said CDR3 having the amino acid sequence SEQ ID NO:94. Furthermore the second part of the IL-18/IL-1β bispecific antibody is selected from a human IL-1β antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:44, said CDR2 having the amino acid sequence SEQ ID NO:45, and said CDR3 having the amino acid sequence SEQ ID NO:46; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO:60, said CDR2 having the amino acid sequence SEQ ID NO:61, and said CDR3 having the amino acid sequence SEQ ID NO:62.

In one embodiment, the first part of the IL-18/IL-1β bispecific antibody for use in treatment or prevention of HS, is selected from a human IL-18 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:76, said CDR2 having the amino acid sequence SEQ ID NO:77 and said CDR3 having the amino acid sequence SEQ ID NO:78; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO:92, said CDR2 having the amino acid sequence SEQ ID NO:93, and said CDR3 having the amino acid sequence SEQ ID NO:94. Furthermore, the second part of the IL-18/IL-1β bispecific antibody is selected from a human IL-1β antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR 1 having the amino acid sequence SEQ ID NO:44, said CDR2 having the amino acid sequence SEQ ID NO:45 and said CDR3 having the amino acid sequence SEQ ID NO:46; and b) an immunoglobulin light chain or fragment thereof which comprises a variable domain comprising, in sequence, the hypervariable regions CDR1, CDR2, and CDR3 and the constant part or fragment thereof of a human light chain, said CDR1 having the amino acid sequence SEQ ID NO:60, said CDR2 having the amino acid sequence SEQ ID NO:61, and said CDR3 having the amino acid sequence SEQ ID NO:62.

The first V_{H1} or V_{L1} domain of an IL-18/IL-1β bispecific antibody used in the disclosed methods may have a first V_{H1} and/or first V_{L1} domains that are substantially identical to the V_{H} or V_{L} domains set forth in SEQ ID NO:85 and 101. An IL-18/IL-1β bispecific antibody for use in treatment or prevention of HS, as disclosed herein may comprise a first heavy chain that is substantially identical to that set forth as SEQ ID NO:87 and/or a first light chain that is substantially identical to that set forth as SEQ ID NO: 103. An IL-18/IL-1β bispecific antibody for use in treatment or prevention of HS, as disclosed herein may comprise a first heavy chain that comprises SEQ ID NO:87 and a first light chain that comprises SEQ ID NO:103. An IL-18/IL-1β bispecific antibody for use in treatment or prevention of HS may comprise: a) a first heavy chain, comprising a variable domain having an amino acid sequence substantially identical to that shown in SEQ ID NO:85 and the constant part of a human heavy chain having a hetero-dimerization modification; and b) a first light chain, comprising a variable domain having an amino acid sequence substantially identical to that shown in SEQ ID NO: 101 and the constant part of a human light chain. The constant part of the human heavy chain may be IgG1. In one embodiment, the IgG1 is a human IgG1 without effector mutations. In one embodiment, the human heavy chain IgG1 comprising a silencing mutation N297A, D265A or a combination of L234A and L235A. In one specific embodiment, the human heavy chain IgG1 comprises the silencing mutation which is a combination of L234A and L235A, according to SEQ ID NO:87.

The second V_{H2} or V_{L2} domain of an IL-18/IL-1β bispecific antibody used in the disclosed methods may have a second V_{H2} and/or first V_{L2} domains that are substantially identical to the V_{H} or V_{L} domains set forth in SEQ ID NO:53 and 69. An IL-18/IL-1β bispecific antibody for use in treatment or prevention of HS, as disclosed herein may comprise a second heavy chain that is substantially identical to that set forth as SEQ ID NO:55 and/or a second light chain that is substantially identical to that set forth as SEQ ID NO:71. An IL-18/IL-1β bispecific antibody for use in treatment or prevention of HS as disclosed herein may comprise a second heavy chain that comprises SEQ ID NO:53 and a second light chain that comprises SEQ ID NO:69. An IL-18/IL-1β bispecific antibody for use in treatment or prevention of HS as disclosed herein may comprise: a) a second heavy chain, comprising a variable domain having an amino acid sequence substantially identical to that shown in SEQ ID NO:53 and the constant part of a human heavy chain having a hetero-dimerization modification, which is complementary to the hetero-dimerization of the first heavy chain; and b) a second light chain, comprising a variable domain having an amino acid sequence substantially identical to that shown in SEQ ID NO:69 and the constant part of a human light chain. The constant part of the human heavy chain may be IgG1. In one embodiment, the IgG1 is a human IgG1 without effector mutations. In one embodiment, the human heavy chain IgG1 comprising a silencing mutation N297A, D265A or a combination of L234A and L235A. In one specific embodiment, the human heavy chain IgG1 comprises the silencing mutation which is a combination of L234A and L235A, according to SEQ ID NO:55.

Other preferred IL-18 antagonists (e.g. antibodies) for use as the first part of a bispecific antibody in the disclosed methods, kits and regimens are those set forth in U.S. Pat. No. 9,376,489, which is incorporated by reference herein in its entirety.

Other preferred IL-1β antagonists (e.g. antibodies) for use as the second part of a bispecific in the disclosed methods, kits and regimens are those set forth in US Pat. Nos: 7,446, 175 or 7,993,878 or 8,273,350, which are incorporated by reference herein in their entirety.

### Example 4: In Vitro Activity of bbmAb1

Binding activity of bbmAb1 was tested in a variety of different cell assays.

### (1) Materials and Methods

### (a) For Solution Equilibrium Titration (SET) Assays

The following material was used:
Recombinant human IL-18, biotinylated (BTP25828)
Recombinant Cynomolgus monkey IL-1β (Novartis)
Anti-human IgG antibody, SULFO-TAG labeled (Meso Scale discovery (MSD) #R32AJ-5). Goat anti-human Fab specific, conjugated with MSD SULFO-TAG NHS Ester (Jackson Immuno Research #109-005-097, MSD #R91AN-1) BSA (Sigma #A-9647)
MSD read buffer T with surfactant (MSD #R92TC-1)
Phosphate-buffered saline (PBS) 10× (Teknova #P0195) Tris-buffered saline, pH 7.5 (TBS) 10× (Teknova #T1680) Tween-20 (Fluka #93773)
Polypropylene microtiter plate (MTP) (Greiner #781280)
384-well plates, standard (MSD #L21XA)

### (b) For Cellular Assays and SET Assays

mAb2 as described in section IL-1β antibody.
mAb1 as described in section IL-18 antibody.
bbmAb1 as described in Example 1.
Recombinant human IL-18 (BTP 25829) purchased from MBL Int. Corp. (#B001-5)
Recombinant marmoset IL-1β (Novartis)
Recombinant marmoset IL-18 (Novartis)
Recombinant human IL-12 (#573008) was purchased from Biolegend KG-1 cell line (ATCC #CCL-246)
Normal human dermal fibrobasts (#CC-2509) were purchased from Lonza Marmoset skin fibroblasts (#42637F (510))
HEK-Blue™ IL-18/IL-1β cells (#hkb-il18) were purchased from InvivoGen PBMC were isolated from buffy coats were obtained from the Blutspendezentrum Bern Marmoset blood was obtained from SILABE, Niederhausbergen
IL-6 ELISA: Human (BioLegend, #430503); Marmoset (U-CyTech biosciences, CT974-5)
IFNγ ELISA: Human (BD555142) and marmoset (U-CyTech biosciences #CT340A)
QUANTI-Blue™ assays (#rep-qb1) for the detection of SEAP was purchased from InvivoGen
Cell medium: RPMI 1640 (Invitrogen #31870) supplemented with 10% Foetal Bovine Serum (Invitrogen #10108-157), 1% L-Glutamine (Invitrogen #25030-03), 1% penicillin/streptomycin (Invitrogen #15140-148), 10 μM 2-Mercaptoethanol (Gibco #31350-010), 5 mM Hepes (Gibco #15630-080)
Round-bottomed, tissue-culture treated 96-well plates (Costar #3799)
Flat-bottomed, tissue-culture treated 96-well plates (Costar #3596)
Ficoll-Pacque™ Plus (GE Healthcare Life Sciences #17-1440-02) PBS 1×, without Calcium & Magnesium (Gibco #14190094)
Leucosep tubes with porous barrier, 50 ml, polypropylene (Greiner bio-one #227290)
Falcon 15 ml polypropylene conical tubes (BD #352096)
Falcon 50 ml polypropylene conical tubes (BD #352070)

### (c) Affinity Measurements by SET

### SET Individual Target Binding Assay

22 serial 1.6n dilutions of the antigens (highest conc.: hulL-18, 5 nM; marIL-18, 10 nM; hulL-13, 0.5 nM; marIL-13, 0.5 nM) were prepared in sample buffer (PBS containing 0.5% Bovine serum albumin (BSA) and 0.02% Tween-20) and a constant concentration of antibody was added (for IL-18 readout 10 pM, for IL-1β readout 1 pM). A volume of 60 ul/well of each antigen-antibody mix was distributed in duplicates to a 384-well polypropylene microtiter plate (MTP). Sample buffer served as negative control and a sample containing only antibody as positive control (Maximal electrochemiluminescence signal without antigen, Bₘₐₓ). The plate was sealed and incubated overnight (o/n, at least 16 h) at room temperature (RT) on a shaker.

IL-18 readout: A streptavidin coated 384-well MSD array MTP was coated with 30 μl/well biotinylated hulL-18 (0.1 μg/ml, PBS) and incubated for 1 h at RT on a shaker. IL-1β readout: A standard 384-well MSD array MTP was coated with 30 μl/well of hull-1 (3 μg/ml, PBS) diluted in PBS as capture agent and incubated overnight at 4° C.

The plate was blocked with 50 μl/well blocking buffer (PBS containing 5% BSA) for 1 hour (h), at room temperature (RT). After washing (TBST, TBS containing 0.05% Tween 20), a volume of 30 μl/well of the equilibrated antigen-antibody mix was transferred from the polypropylene MTP to the coated MSD plate and incubated for 20 min at RT. After an additional wash step, 30 μl sulfo tag-labeled anti-IgG detection antibody (0.5 μg/ml) diluted in sample buffer were added to each well and incubated for 30 min at RT on a shaker. The MSD plate was washed and 35 μl/well MSD read buffer were added and incubated for 5 min at RT. Electrochemiluminescence (ECL) signals were generated and measured by the MSD Sector Imager 6000.

### SET Simultaneous Target Binding Assays

The SET assay was performed a described above, except for Assay A: The equilibration process (antibody/antigen mix) was performed in presence of an excess of one target (500 pM of either IL18 or IL-1β) while assessing the K_{D} of the other target. Assay B: The equilibration process (antibody/antigen mix) was performed with both targets in serial dilutions in one mix simultaneously (constant concentration of antibody 10 pM, highest antigen conc. see above). The same mix was then analyzed for its free antibody concentration on IL18 and IL-1β coated plates as described above.

The SET Data were exported to XIfit, an MS Excel add-in software. Average ECL-signals were calculated from duplicate measurements within each assay. Data were baseline adjusted by subtracting the lowest value from all data points and plotted against the corresponding antigen concentration to generate titration curves. K_{D} values were determined by fitting the plot with the following:

### 1:2 Binding Model for the Monospecific Ab

$$y = \frac{2 \text{?}}{[ IgG ]} ( \frac{[ IgG ]}{2} - \frac{{( \frac{x + [ IgG ] + {K}_{D}}{2} - \sqrt{\frac{{( x + [ IgG ] + {K}_{D} )}^{3}}{4} - x [ IgG ]} )}^{2}}{2 [ IgG ]} )$$

$$\text{?} \text{indicates text missing or illegible when filed}$$

### 1:1 Binding Model for the Knob in Hole Bispecific Ab

$$y = {B}_{max} - ( \frac{{B}_{max}}{2 [ Fab ]} ( [ Fab ] + x + {K}_{D} - \sqrt{{( [ Fab ] + x + {K}_{D} )}^{2} - 4 x [ Fab ]} ) )$$

wherein
y: blank subtracted ECL signal
Bₘₐₓ: maximal ECL signal at zero antigen concentration
[IgG]: applied antibody concentration
[Fab]: applied total Fab concentration
K_{D}: Dissociation equilibrium constant
x: applied antigen concentration

### (d) Cell Culture

KG-1 cells were grown in RPMI 1640 supplemented with 10% fetal bovine serum, 1% L-Glutamine and 1% penicillin/streptomycin at a density of 2×10⁵ to 1×10⁶ viable cells/mL.

Normal human fibroblasts and marmoset fibroblasts were grown in FBM (Clonetics, CC-3131) including bFGF (1 ng/ml, CC-4065), insulin (5 μg/ml, CC-4021), and 2% FCS (CC-4101). As starving medium, Fibroblast Basal Medium (LONZA #CC-3131) was used. HEK-Blue™ IL-18/IL-1β cells were grown in Growth Medium (DMEM, 4.5 g/l glucose, 10% (v/v) fetal bovine serum, 50 U/ml penicillin, 50 mg/ml streptomycin, 100 mg/ml Normocin™, 2 mM L-glutamine supplemented with 30 μg/ml of Blasticidin, 200 μg/ml of HygroGold™ and 100 μg/ml of Zeocin™

Human peripheral blood mononuclear cells (PBMC) were freshly isolated from buffy coats using LeucoSep tubes according to the instructions of the manufacturer. In brief, 13 ml of Ficoll-Paque was preloaded in a 14 ml LeucoSep tube by centrifugation for 30 s at 1,000×g. The heparinized whole-blood samples were diluted with equal volumes of PBS, and 25 ml of the diluted blood was added to a LeucoSep tube. The cell separation tubes were centrifuged for 15 min at 800× g without break at room temperature. The cell suspension layer was collected, and the cells were washed twice in PBS (for 10 min at 640 and 470× g, respectively, for the two successive washes) and re-suspended in culture medium before counting.

Marmoset blood was collected in heparinized tubes and filtered using a 70 μm cell strainer (BD Biosciences #352350)

### (e) IL-13 Neutralization Assays

The IL-1β induced IL-6 production assay in fibroblasts was conducted essentially as described (Gram 2000) with only minor modifications. Briefly, fibroblasts were seeded at a density of 5×103 cells per well (in 100 μl) in a 96 well flat bottom tissue culture plate. The following day, cells were starved for 5h in starving medium before addition of the recombinant IL-1β/compound solution mix (IL-1β concentration indicated in the table). The IL-1β/compound solution mix was prepared beforehand by incubating recombinant IL-1β with a concentration range of compound for 30 min at 37° C. The cell supernatants were collected after o/n incubation at 37° C. and the amount of released IL-6 determined by ELISA. The IL-1β induced IL-6 production assay in PBMC was performed according to the following. PBMC were seeded at 3×10⁵ cells per well (in 100 μl) in a 96 well tissue culture plate and incubated with a recombinant IL-1β/compound solution mix for 24h at 37° C. (IL-1β concentration indicated in the table). The IL-1β/compound solution mix was prepared beforehand by incubating recombinant IL-1β with a concentration range of compound for 30 min at 37° C. The cell supernatants were collected after 24h of stimulation and the amount of released IL-6 determined by ELISA.

### (f) IL-18 Neutralization Assays

The assay was conducted essentially according to the following. KG-1 cells (starved for 1h in PBS+1% FCS beforehand) or PBMC at a density of 3×10⁵ per well were seeded into round bottom 96-well cell culture plates and incubated with a solution mix of recombinant IL-18/IL-12 together with a concentration range of compounds (IL-18/IL-12 concentrations indicated in the table). After an incubation of 24h at 37° C., supernatants were collected and the amount of released IFNγ determined by ELISA. For the assays with marmoset blood 85 μl of blood per well were used.

### (g) Dual IL1β/IL-18 Neutralization in HEK-Blue™ Cells

The assay was conducted essentially as described in the manufacturer's handling procedures. Briefly, the HEK-Blue™ cells were seeded at a density of 4×10⁴ per well into 96-well cell culture plates and incubated with a solution mix of recombinant IL-1β and IL-18 (to produce a 1:1 SEAP signal) together with a concentration range of compounds. After an incubation of 24h at 37° C., supernatants were collected and the amount of released SEAP determined by using the QUANTI-Blue™ method according to the manufacturer's instructions.

All Data were exported to EXCEL software and IC50 values calculated by plotting dose-response curves for the logistic curve fitting functions using either EXCEL/XLfit4 or GraphPad Prism software.

### (2) Results

### (a) Affinities to Recombinant Human and Marmoset IL1B and IL-18

Binding affinities of bbmAb1 to human and marmoset recombinant IL-1β and IL-18 proteins were measured by solution equilibrium titration (SET) titration and the K_{D} values generated were compared to those of mAb2 for IL-1β and mAb1 for IL-18 binding.

Comparing binding affinities in the individual target binding assay, bbmAb1 showed a similar mean KD compared to mAb1 for human and marmoset IL-18 (Table 7). For human IL-1β binding the mean KD value was slightly higher for bbmAb1 (2.6 pM) compared to mAb2 (0.6 pM) but still in the same low pM range. Subsequent measurements in the simultaneous dual target binding assay (Table 8) confirmed that bbmAb1 binding KD values for IL-1β were similar to values of mAb2 with the pre-clinical as well as with the clinical grade material. Thus, bbmAb1 possesses binding affinities for both targets in humans and marmosets that are in similar to mAb2 and mAb1, respectively.

TABLE 7
Affinities to recombinant human (hu) and marmoset (mar) IL-1β
and IL-18 measured by SET (individual target binding determination)

| | Independent IL-18/IL-1β affinity determination | | | |
|---|---|---|---|---|
| | huIL-18 K_{D} | marIL-18 K_{D} | huIL-1β K_{D} | marIL-1β K_{D} |
| Samples | [pM] | [pM] | [pM] | [pM] |
| mAb1 | 9 ± 2 | 21 ± 3 | n/a | n/a |
| mAb2 | n/a | n/a | 0.6 ± 0.1 | 1.0 ± 0.7 |
| bbmAb1 | 12 ± 4 | 33 ± 7 | 2.6 ± 0.1 | 3.0 ± 2.4 |

In addition to the individual target binding results, simultaneous dual target binding affinities of bbmAb1 were investigated (Table 8) by applying either excess of one target during the assessment of the binding the K_{D} values of the other target (Assay A) or by applying a mixture of both targets in serial dilutions (Assay B). Simultaneous IL-1β/IL-18 affinity determination showed no significant difference between Assay A (excess of one antigen) and Assay B (mixture of both antigens in serial dilutions) which proved that both targets are bound simultaneously without affecting the binding of the other target. Furthermore, the K_{D} values obtained with the simultaneous dual binding assays were similar to the K_{D} values obtained with the standard assay (Table 7); in the absence of the second antigen) which proved that bbmAb1 can bind both antigens independently. Thus, bbmAb1 binds simultaneously and independently both human IL-1β and IL-18 and fully cross-reacts with the corresponding marmoset proteins.

TABLE 8
Affinities to recombinant human (hu) and marmoset (mar) IL-1β
and IL-18 measured by SET (simultaneous target binding determination

| | Simultaneous IL-18/IL-1β affinity determination | | | | | | | |
|---|---|---|---|---|---|---|---|---|
| | huIL-18 K_{D} [pM] | | marIL-18 K_{D} [pM] | | huIL-1β K_{D} [pM] | | marIL-1β K_{D} [pM] | |
| Samples | Assay A | Assay B | Assay A | Assay B | Assay A | Assay B | Assay A | Assay B |
| mAb1 | 13.5 | 11.4 | 27.1 | 26.3 | n/a | No | n/a | No |
| | | | | | | binding | | binding |
| mAb2 | n/a | No | n/a | No | 1.1 | 3.2 | 0.8 | 4.8 |
| | | binding | | binding | | | | |
| bbmAb1 | 14.8 | 19.5 | 47.9 | 44.2 | 3 | 0.5 | 2 | 0.6 |

(b) Neutralizing Activity of bbmAb1 in Human and Marmoset Cell Assays

The neutralizing activity of bbmAb1 for both cytokines (IL1β and IL-18) was assessedmAb2mAb1). In addition, the potency of bbmAb1 for the neutralization of marmoset IL-1β and IL-18 using marmoset cell assay systems was assessed (see section d).

### (c) Individual and Simultaneous IL-1β and IL-18 Neutralization in Human Cells

The neutralizing activity of bbmAb1 on IL-1β was assessed by the inhibition of recombinant IL-1β-induced IL-6 production in human dermal fibroblasts (IL-1β used at 6 pM) and in human PBMC (IL-1β used at 60 pM). The neutralizing activity of bbmAb1 on IL-18 was measured by the inhibition of recombinant IL-18-induced IFN-γ production in KG-1 cells and human PBMC (both cells activated with 3 nM recombinant human IL-18 together with 1 ng/ml of recombinant human IL-12). The inhibitory potency of bbmAb1 on IL-1β and IL-18 was always compared to that of either mAb2 or mAb1, respectively. Depending on the assays, the mean IC50 values of bbmAb1 were in sub-nM or single digit nM ranges and up to 2-to 4-fold higher in direct comparison mAb2 (for IL-1β) and mAb1 (for IL-18), respectively (Table 9 and Table 10). The monovalent format of bbmAb1 as compared to the bivalent format of mAb2/mAb1 but also potentially the KiH mutations may be reasons for this slight difference in potency of bbmAb1.

TABLE 9
Mean IC50 values for IL-1β neutralization by bbmAb1 in
comparison to mAb2 in human dermal fibroblasts and human PBMC.

| | IL-6 prod.* derm. fibrobl. | IL-6 prod.* PBMC |
|---|---|---|
| IL-1β inhibition | IC₅₀ [nM] | IC₅₀ [nM] |
| mAb2 | 0.031 ± 0.006 | 0.29 ± 0.67 |
| bbmAb1 | 0.136 ± 0.045 | 1.35 ± 0.59 |

*Inhibition of IL-6 production in human dermal fibroblasts or PBMC stimulated with recombinant human IL-1β (6 pM for dermal fibroblasts and 60 pM for PBMC). Shown are mean values ± SEM (n = 3 PBMC and n = 6 human dermal fibroblasts)

TABLE 10
Mean IC50 values for IL-18 neutralization by bbmAb1
in comparison to mAb1 in KG-1 cells and human PBMC.

| | IFNγ prod.** KG-1 cells | IFNγ prod.** PBMC |
|---|---|---|
| IL-18 inhibition | IC₅₀ [nM] | IC₅₀ [nM] |
| mAb1 | 0.035 ± 0.011 | 0.78 ± 0.49 |
| bbmAb1 | 0.071 ± 0.046 | 0.87 ± 0.51 |

**Inhibition of IFNγ production in KG-1 cells or PBMC stimulated with recombinant human IL-18 (3 nM) and human IL-12 (1 ng/ml). Shown are mean values ± SEM (n = 3 KG-1 and n = 4 PBMC)

bbmAb1 was able to neutralize simultaneously the bioactivity of both IL-1β and IL-18 as demonstrated with the HEK Blue™ reporter cells producing SEAP in response to a 1+1 stimulation with recombinant IL-1β and IL-18 (Table 11). A similar inhibition of SEAP in this assay system was only achievable by the combination of mAb2 and mAb1 but not by the use of the individual antibodies.

TABLE 11
Mean IC50 values for simultaneous neutralization of IL-
1β and IL-18 on SEAP reporter activity in HEK Blue ™ cells.
Shown are means ± SEM of n = 5 experiments.

| Inhibition of SEAP in HEK | |
|---|---|
| reporter cells stimulated simultaneously | IC₅₀ |
| with IL-1β and IL-18 | [nM] |
| mAb2 or mAb1 alone | >30 |
| mAb2 and mAb1 combined | 0.24 ± 0.09 |
| bbmAb1 | 0.71 ± 0.28 |

(d) Neutralizing Activity of bbmAb1 on Marmoset IL-18 and Marmoset IL-18 in Marmoset Cell Assays

In order to demonstrate the inhibitory activity of bbmAb1 in marmoset, similar in vitro assays were performed with marmoset cells as with human cells however using recombinant marmoset IL-1β and IL-18 for stimulation. When assessing the inhibition of recombinant marmoset IL-1β-induced IL-6 production in marmoset dermal fibroblasts, bbmAb1 displayed sub-nM potency with 2-to 3-fold higher IC50 values compared to mAb2 (Table 12). Testing bbmAb1 with human dermal fibroblasts stimulated with marmoset IL-1β generated a similar inhibition profile as with human IL-6.

TABLE 12
Inhibition of recombinant marmoset IL-1β induced
IL-6 production in marmoset and human fibroblasts by bbmAb1.

| | | |
|---|---|---|
| | IL-6 prod.* marmoset | IL-6 prod.* human |
| z | dermal fibroblasts | derm. fibroblasts |
| Marmoset | IC₅₀ [nM] | IC₅₀ [nM] |

| IL-1β | Exp. A | Exp. B | Exp. C |
|---|---|---|---|
| bbmAb1 | 0.174 | 0.364 | 0.220 |
| mAb2 | 0.095 | 0.138 | 0.114 |

*Inhibition of IL-6 production in marmoset or human dermal fibroblasts stimulated with recombinant marmoset IL-1β (18 pM). Results of 3 individual experiments (A, B and C) are shown.

Single to double digit nM IC50 values of bbmAb1 confirmed the neutralizing activity of bbmAb1 for marmoset IL-18 tested in the IFNγ production assay with marmoset blood cells (Table 13). Testing bbmAb1 with human PBMC stimulated with marmoset IL-18 generated a similar inhibition profile when measuring the production of human IFNγ.

Thus, bbmAb1 was shown to be fully cross-reactive to marmoset IL-1β and marmoset IL-18 in functional assays using marmoset responder cells.

TABLE 13
Mean IC50 values for inhibition of recombinant marmoset IL-18
induced IFNγ production in marmoset whole blood or human PBMC.

| | IFNγ prod.** | IFNγ prod.** | Marmoset | |
|---|---|---|---|---|
| Marmoset | Marmoset blood | Human PBMC | IL-18 | |
| IL-18 | IC₅₀ [nM] | IC₅₀ [nM] | conc. used | |
| bbmAb1 | 10.0 ± 4.1 | | 1 | nM |
| mAb1 | 4.7 ± 2.6 | | 0.3 | nM |
| mAb1 | 181 ± 108 | | 3 | nM |
| mAb1 | | 6.6 ± 5.0 | 1 | nM |

**Inhibition of IFNγ production in marmoset whole blood (n = 3 each compound/condition) or human PBMC (n = 6) stimulated with recombinant marmoset IL-18 (concentration indicated) & human IL-12 (10 ng/ml). Shown are mean values ± SEM

It was demonstrated that bbmAb1, a KiH format IL-1β/IL-18 bi-specific mAb retains the high affinity binding as well as the cytokine neutralizing potency to the two individual targets IL-1β and IL-18 when compared to the original mAbs, mAb2 and mAb1, in a variety of different cell assays. The dual IL-1β and IL-18 neutralizing properties of bbmAb1 were not only demonstrated for the human cytokines/cells but also for the corresponding marmoset cytokines/cells, facilitating appropriate toxicology studies. The up to 2-to 4-fold higher IC50 values that were generated in some of the cellular assays for IL-1β and IL-18 neutralization may be the consequence of the monovalent binding of bbmAb1 as opposed to bi-valent binding of mAb2 and mAb1, respectively. Nevertheless, the dual cytokine neutralization by bbmAb1 may result in additive or synergistic inhibitory activities in vivo that may not be adequately represented in our in vitro cellular systems.

### Example 5: Effects of Combined IL-1β and IL-18 Stimulation and Blockade in PBMC

Inflammasome activation-dependent cleavage of the effector cytokines IL-1β and IL-18 leads to the induction of secondary pro-inflammatory mediators and promotes immune cell recruitment/activation not only systemically but also at the site of inflammation. In two different mouse models for lethal systemic inflammation (a) LPS injection model and (b) FCAS mice (activating missense mutations in NLRP3), the simultaneous absence/inhibition of both IL-1β and IL-18 was more protective from lethality compared to the single IL-1β or single IL-18 absence/inhibition, demonstrating additive or synergistic mechanisms for immune activation (Brydges 2013, van den Berghe 2014). bbmAb1 is a human/marmoset IL-1β/IL-18 reactive bi-specific mAb with no rodent cross-reactivity and thus cannot be tested in mouse models. Therefore, we used LPS/IL-12 to mimic inflammasome-dependent pathway activation in vitro for the stimulation of human PBMC to reveal additive or synergistic inhibitory effects of combined IL-1β/IL-18 neutralization by bbmAb1 and performed a non-biased gene expression analysis using microarrays. As a complementary activity we also compared the gene expression profiles of PBMCs from different donors stimulated with either the combination of recombinant IL-1β and recombinant IL-18 or the single cytokines alone.

### (3) Materials and Methods

### (a) Cell Culture and ELISA

RPMI 1640 (Invitrogen #31870 or Gibco #61870-010) supplemented with 10% Foetal Bovine Serum (Invitrogen #10108-157), 1% L-Glutamine (Invitrogen #25030-03), 1% penicillin/streptomycin (Invitrogen #15140-148), 10 μM 2-Mercaptoethanol (Gibco #31350-010), 5 mM Hepes (Gibco #15630-080)
Recombinant Human IL-1β was purchased from Sino Biological Inc. (#10139-HNAE-5)
Recombinant human IL-18 was purchased from MBL (#B001-5)
Recombinant human IL-12 was purchased from Biolegend (#573008)
IFNγ ELISA: MAX Standard Set, BioLegend, #430103 or BD OptEIA human IFNγ ELISA Set, BD #555142
IL-6 ELISA: MAX Standard Set, BioLegend, #430503
IL-26 ELISA: Cloud Clone Corp #SEB695Hu
mAb2 as described in section IL-1β antibody.
mAb1 as described in section IL-18 antibody.
bbmAb1 as described in Example 1.
LPS from Salmonella enterica serotype enteritidis, Sigma #L7770
PBMC were isolated from buffy coats that were obtained from the Blutspendezentrum Bern Round-bottomed, tissue-culture treated 96-well plates (Costar #3799) Flat-bottomed, tissue-culture treated 96-well plates (Costar #3596) Ficoll-Pacque™Plus (GE Healthcare Life Sciences #17-1440-02) PBS 1X, without Calcium & Magnesium (Gibco #14190094) Falcon 15 ml polypropylene conical tubes (BD #352096) Falcon 50 ml polypropylene conical tubes (BD #352070)
Leucosep™ tubes with porous barrier, 50 ml, Greiner bio-one #227290
Cell strainer 70 μM, BD Biosciences #352350
Trypanblue, Sigma #T8154
RNA isolation, quantity and quality measurements and qPCR:
Nuclease-free water, Ambion #AM9938
Rnase Zap, Ambion #AM9780
1.5 ml Eppendorf tubes, sterile, Rnase & Dnase free
RLT buffer, Qiagen #1015762
Rneasy Mini Kit, Qiagen #74104
RNase-Free DNase Set, Qiagen #79254
Agilent RNA 6000 Nano Kit, Agilent #5067-1511
Chip priming station, Agilent #5065-4401
IKA vortex mixer
RNaseZAPR, Ambion #9780
Agilent 2100 Bioanalyzer
High Capacity cDNA reverse transcription kit, Applied Biosystems, #PN4374966
Nase-free, Thin-Walled, forsted Lid 0.2 ml PCR tubes, Ambion #AM12225
MicroAmp Optical 384 well reaction plate, Applied Biosystems #4309849
TaqMan GenEx Master Mix, Applied Biosystems #4369514
PCR primer (Applied Biosystems)

| Target | Assay ID Taqman | color/quencher |
|---|---|---|
| IFNγ | Hs00989291_m1 | FAM-MGB |
| IL-26 | Hs00218189_m1 | FAM-MGB |
| RPL27 | Hs03044961_g1 | FAM-MGB |
| HPRT1 | Hs02800695_m1 | FAM-MGB |

PBMC preparation: PBMCs were isolated from buffy coat by means of Ficoll-Paque gradient centrifugations in Leucosep tubes according to the manufacturer's instructions. Briefly, 15 mL of Histopaque was put in 50 mL Leucosep™ tubes and centrifuged for 30 sec at 1300 rpm at RT. With a pipette, 30 mL of a diluted suspension of the buffy coat was added on the top of the Histopaque solution and centrifuged during 15 min at RT at 1000 g without break. Plasma was discarded (approx. 20 ml) and the interface ring collected (=human PBMC) and transferred in a 50 ml falcon tube. The tube was filled with 50 mL of sterile PBS and centrifuged once at 1200 rpm during 5 min at RT. This centrifugation was repeated 2 times. The supernatant was gently discarded and cells re-suspended in 50 mL of PBS with 2% FCS and 2 mM EDTA. The cell suspension was filtered using a 70 μm cell strainer and cells counted using trypan blue staining (500 μL of trypan blue+200 μL of cells+300 μL of PBS).

LPS/IL-12 stimulation of PBMC: Cytokine production in supernatants was prepared according to the following. 250 000 cells/well in 100 ul final volume were distributed in 96-well round bottom plates. LPS was used at concentrations between 0.3 μg/ml and 3000 μg/ml together with recombinant IL-12 at 10 ng/ml. Supernatants were harvested after 24h at 37° C. and 10% CO₂.

RNA extraction from cell pellets was performed according to the following. 3×10⁶ cells/well in 1000 ul final volume were distributed in flat bottom 24-well plates. LPS was used at 3 μg/ml together with recombinant IL-12 at 10 ng/ml. Cells were harvested after 24h at 37° C. and 10% CO₂.

Stimulation of PBMC with recombinant cytokines: 7×10⁶ PBMC per well of a 12-well plate were used in 1.5 ml final of complete RPMI medium. Recombinant cytokines were added at the following final concentrations: 10 ng/ml of recombinant IL-1β, 3 nM of recombinant IL-18, 1 ng/ml of recombinant IL-12. Both, supernatants as well as cells were collected after 4h and 24h at 37° C. and 10% CO₂.

RNA isolation, quantity and quality assessments: Cells were pelleted and the pellet lysed in 350 μl of Qiagen RTL buffer with 2% B-mercaptoethanol and frozen at −20° C. or −80° C. until all samples of the study have been collected. The RNA isolation was performed using the Qiagen standard protocol. Briefly, 350 μl of 70% Ethanol was added in all samples prior to the transfer to the RNeasy spin column and centrifuged for 15 s at 8000 g. After discarding the flow-through, 350 μl of buffer RW1 was added and the column centrifuged for 15 s at 8000 g to wash the spin column membrane. DNase I incubation mix solution was prepared according to the manufacturer's instructions and added to the RNeasy spin column and incubated for 15 min at RT. After washes with 350 μl and 500 μl of buffer RW1, the RNeasy spin column was placed in new 2 ml collection tube and centrifuged at full speed for 1 min. RNA was finally collected by adding 35 μl RNase-free water directly to the spin column membrane and a centrifugation for 1 min at 8000 g to elute the RNA. The amount of RNA was measured using Nanodrop ND-1000 and the RNA was stored at −20° C. RIN measurements were performed for the RNA quality assessment according to manufacturer's instructions. Briefly 1 μl of RNA or ladder were pipetted into an Agilent RNA 6000 Nano chip and measured by using the Agilent 2100 Bioanalyser.

Cytokine Gene Expression Analysis by qPCR:

The method was performed corresponding to the manufacturer's instructions. Briefly, 400 ng of RNA was reverse transcribed according to the instructions using the High-Capacity cDNA Reverse Transcription Kit. The cDNA solutions were diluted 1/10 in RNA/DNA free water and 1 μl cDNA was transferred into a 384-well reaction plate and then mixed with 1 μl of 20× TaqMan® Gene Expression Assay target FAM gene and 10 μl of 2× TaqMan® Gene Expression Master Mix and 10 μl RNA/DNA free water. The plate was loaded onto the Applied Biosystems ViiA™ 7 Real-Time PCR System and the following instrument settings were used:

| | |
|---|---|
| Plate document/ | Thermal cycling conditions |

| experiment | | Temp | Time |
|---|---|---|---|
| parameters | Stage | (° C.) | (mm:ss) |
| Rxn. Volume: 20 μL | Hold | 50 | 2:00 |
| Ramp rate: Fast | Hold | 95 | 0:20 |
| | Cycle | 95 | 0:01 |
| | (40 cycles) | 60 | 0:20 |

The house keeping genes used for this study were HPRT1 and RLP27. The following formula was used to calculate the relative expression levels of target genes:

$$Ct [ Ref ] = ( C t [ HPRT 1 ] + C t [ RLP 27 ] ) / 2 \qquad 1 )$$

$$dCt [ Ref ] = 40 - Ct [ Ref ] \qquad 2 )$$

$$dCt [ Target ] = Ct [ Target ] - Ct [ Ref ] \qquad 3 )$$

$$ddCt = dCt [ Ref ] - dCt [ Target ] \qquad 4 )$$

$$Relative target gene expression = 2 \hat{} ddCt \qquad 5 )$$

Microarrays was performed according to the following. Samples were processed by CiToxLAB France on Affymetrix HG_U133_Plus2 microarrays. They were RMA normalized and analyzed in GeneSpring 11.5.1 (Agilent Technologies, Santa Clara, CA). Pathway analysis was done using Ingenuity Pathway Analysis (IPA) and Nextbio (Illumina). The two datasets were treated independently.

Initially, the data were subject to standard quality control (QC) by CiToxLAB, in-house QC by using an R script (MA_AffyQC.R) in Rstudio suite and in GeneSpring (PCA, hybridization controls). Subsequently, it was filtered to eliminate unreliable expression levels: Entities (probesets) were kept where at least 100 percent of samples in any 1 of the experimental conditions have values above the 20th percentile.

Differentially expressed genes (DEG) were identified using the “filter on volcano plot” feature in GeneSpring. Using the filtered genes (expression between 20.0-100.0th percentiles) with an unpaired T-test, probesets with a corrected p-value below 0.05 and a fold change above 2.0 were considered differentially expressed. Where possible, i.e. in the study with LPS (NUID-0000-0202-4150) a Benjamini-Hochberg Multiple Testing Correction was used.

For cytokine stimulation experiments, synergism was calculated using the following formula: Signal A+B/(Signal A+Signal B-Control)≥1.5

The respective signatures (or DEG lists) were used to calculate p-values with a Fisher's exact test which represent the statistical significance of observing an overlap between the signature and the ‘disease gene list’ (lesional vs non-lesional) of public datasets. To do so, the lists were uploaded into Illumina Base Space Correlation Engine (former Nextbio) and compared using the Meta-Analysis feature and keyword search for diseases.

All Data were exported to EXCEL software and IC50 values calculated by plotting dose-response curves for the logistic curve fitting functions using either EXCEL/XLfit4 or GraphPad Prism software. Differences between treatment groups were analyzed by one-way ANOVA followed by Dunnett's multiple comparison using GraphPad Prism software and results were considered statistically significant at p<0.05.

### (4) Results

(a) bbmAb1 is Highly Efficacious in Inhibiting LPS/IL-12 Induced IFNγ Production in Whole Blood

Exposure of human whole blood to LP S supplemented with 10 ng/ml IL-12 results in an IFNγ response that is largely but not exclusively dependent on the “native” IL-18 produced by the blood cells. The addition of IL-12 enhances the LPS induced IFNγ responses, likely by up-regulating IL-18 receptors on responder cells.

In the experimental conditions used, IL-18 neutralization with mAb1 lead only to an incomplete inhibition of IFNγ production whereas IL-1β blockade (using mAb2) had only small effects on the IFNγ response. Interestingly, the combined inhibition of IL-1β and IL-18 either by bbmAb1 or the combination of mAb2 and mAb1 was more profoundly and completely inhibiting IFNγ production compared to the single cytokine neutralization. Inhibition of LPS (0.3 μg/ml)/IL-12 induced IFNγ in whole blood by bbmAb1, mAb2, mAb1 or combined mAb2 & mAb1 (Combo).

Apart from IFNγ, none of the other cytokines tested (IL-2,-4,-6,-8,-10,-13 and TNFα) were additively inhibited by the combined neutralization of IL-1β and IL-18 in our cell assay. The potency of bbmAb1 was in the same range as the combination (combo) of mAb2 and mAb1, considering the monovalent format of the bispecific molecule.

(b) IFNγ is Additively Inhibited by bbmAb1 (i.e. Combined IL-1β/IL-18 Inhibition) Compared to Single IL-1β or IL-18 Inhibition in LPS/IL-12 Activated Human PBMC

An unbiased transcriptomics evaluation was required in order to reveal further additive effects (apart of IFNγ) by combined IL-1β/IL-18 inhibition using bbmAb1. Since whole blood is not optimal for transcriptomics analysis we adapted the LPS/IL-12 stimulation assay conditions, described in the materials and method section above, to human PBMC samples. By using PBMCs from a total of 9 donors, we could confirm that bbmAb1 additively inhibited IFNγ protein secretion into the supernatants of the PBMCs. Compared to whole blood experiments, IFNγ production was inhibited at approximatively 10-fold lower concentrations of the respective mAbs used. Importantly, a similar inhibition pattern was demonstrated at the mRNA level for IFNγ which confirmed the suitability of the samples for a non-biased microarray based gene expression analysis. The inhibition of LPS (0.3 μg/ml)/IL-12 induced IFNγ protein production and IFNγ gene expression by bbmAb1, mAb2 and mAb1 (at 10 nM conc. each) in human PBMC was demonstrated.

The Affymetrix microarray was conducted with n=5 individual donors from PBMCs that were sampled from the LPS/IL-12 stimulation experiments described in the materials and method section above. Unfortunately, the overall assessment of the gene expression profiles evidenced a strong LPS/IL-12 stimulation effect and the PCA showed clustering per donor rather than compound within the stimulated or unstimulated groups. Nevertheless, comparing the LPS/IL-12 stimulated samples with the stimulated plus bbmAb1 for differentially expressed genes revealed a shortlist of genes that are downregulated by the combined IL-1β/IL-18 blockade with bbmAb1 (Table 14). Apart from the strong downregulation of the IFNγ gene that re-validated our microarray data, also the IL-26 gene was a further cytokine gene additively inhibited by bbmAb1 compared to the single IL-1β inhibition (by mAb2) or IL-18 inhibition (by mAb1).

TABLE 14
Differentially expressed genes (downregulated genes only between
the bbmAb1 and control group in LPS/IL-12 stimulated samples).

| Probe Set ID | Gene Symbol | Entrez Gene | p-value | FC |
|---|---|---|---|---|
| 222974_at | IL22 | 50616 | 0.03188 | 6.6 |
| 221111_at | IL26 | 55801 | 0.00224 | 5.2 |
| 223939_at | SUCNR1 | 56670 | 0.00234 | 4.0 |
| 1560791_at | OTTHUMG0000010886 | | 0.03660 | 3.7 |
| 211122_s_at | CXCL11 | 6373 | 0.02954 | 3.5 |
| 203915_at | CXCL9 | 4283 | 0.02211 | 3.4 |
| 235229_at | | | 0.02400 | 3.3 |
| 210163_at | CXCL11 | 6373 | 0.02707 | 3.2 |
| 210354_at | IFNG | 3458 | 0.00007 | 2.9 |
| 243541_at | IL31RA | 133396 | 0.01200 | 2.5 |
| 236003_x_at | OR2I1P | | 0.04942 | 2.4 |
| 203131_at | PDGFRA | 5156 | 0.00161 | 2.4 |
| 219991_at | SLC2A9 | 56606 | 0.00191 | 2.4 |
| 201860_s_at | PLAT | 5327 | 0.00139 | 2.3 |
| 205692_s_at | CD38 | 952 | 0.04855 | 2.3 |
| 1555600_s_at | APOL4 | 80832 | 0.02610 | 2.3 |
| 215305_at | PDGFRA | 5156 | 0.01180 | 2.2 |
| 236191_at | | | 0.04037 | 2.1 |
| 204533_at | CXCL10 | 3627 | 0.04847 | 2.1 |
| 229915_at | FAM26F | 441168 | 0.02912 | 2.0 |
| 210072_at | CCL19 | 6363 | 0.02827 | 2.0 |
| 236101_at | | | 0.03246 | 2.0 |

FC = fold change.

(c) IL-26 is Another Pro-Inflammatory Cytokine Additively Inhibited by bbmAb1 in LPS/IL-12 Stimulated PBMC

To further confirm that LPS/IL-12 driven IL-26 gene expression and protein production is most efficiently inhibited by combined IL-1β/IL-18 blockade using bbmAb1, the study was extended to a total of n=9 PBMC donors and investigated IL-26 gene expression by qPCR and IL-26 protein production by ELISA. The ELISA largely confirmed the inhibition of IL-26 gene expression obtained with the microarray approach. Interestingly, IL-26 protein levels in supernatants were only partly reduced at 24h by the addition of the mAbs. The reasons for this differences are unknown, could however be related to kinetic differences between IL-26 gene expression and protein production as well as differences in the consumption of IL-26 compared to IFNγ. Nevertheless, bbmAb1 was superior in reducing IL-26 protein levels in the PBMC supernatants compared to mAb2 and mAb1.

(d) IL1β/IL18 Signaling Signatures Correlate with Disease

Previously established PBMC culture conditions where recombinant IL-1β stimulation resulted in either IL-6 production or recombinant IL-18/IL-12 stimulation resulted in IFNγ production was combined to reveal additive or synergistic downstream target genes or signatures (data not shown). With PBMCs from n=4 donors sampled at two different time points (6h and 24h) an Affymetrix microarray evaluation for unbiased assessment of the gene expression profiles was conducted. Genes that were synergistically upregulated at 6h and at 24 h with the combined stimulation by IL-1β and IL-18 were revealed (data not shown). The addition of IL-12 to the IL-1β/IL-18 combination largely increased the synergy for a series of upregulated genes. The generated signalling signatures of single or combined IL-1β/IL-18 pathway stimulation (UP-regulated genes only) were used to interrogate dataset from patients across several autoimmune diseases. For example, correlation to public sarcoidosis datasets was identified. P-values (calculated with a Fisher's exact test) show a significant correlation to several public studies comparing healthy to diseased tissues from sarcoidosis patients. Tissues include skin as well as lung, lacrimal glands and anterior orbit. Across all datasets, the combination of IL1β/IL18 signaling shows the best correlation to disease, followed by IL-1β and IL-18. IL-1β/IL-18 differentially up-regulated genes (DEG) in PBMC compared to 5 different sarcoidosis tissue ‘diseased vs healthy’ DEG.

### (e) Conclusion

LPS and recombinant IL-12 was used to mimic pathogen associated molecular pattern (PAMP)-dependent NLRP3 inflammasome activation within the first 24h of in vitro culture. It was demonstrated that combined inhibition of IL-1β and IL-18, by using bbmAb1, acts additively to decrease/inhibit IFNγ production in PBMC stimulated with LPS/IL-12. IL-12 was previously described to act synergistically with IL-18 to induce IFNγ production in T, B, NK cells, macrophages and dendritic cells (as reviewed by Nakanishi, 2001) but now an additional stimulatory effect of IL-1β on IFNγ production could be demonstrated under the experimental conditions used. Thus, the co-incubation of PBMC with LPS/IL-12 drives efficiently the production of “native” IL-1β and IL-18 which contribute both to a strong IFNγ response. By using unbiased microarray transcriptomics, additional genes were identified that were additively down-regulated by combined IL-1β/IL-18 neutralization vs. single IL-1β or IL-18 blockade. Amongst those was IL-26, a member of the IL-20 cytokine subfamily (IL-19, IL-20, IL-22, IL-24, and IL-26), which is conserved in most vertebrate species but absent in most rodent strains (including mice and rats) (Donnelly 2010). It signals through a heterodimeric receptor complex composed of the IL-20R1 and IL-10R2 chains. IL-26 receptors are primarily expressed on non-hematopoietic cell types, particularly epithelial cells. Increased levels of IL-26 were reported in serum and particularly in the synovial fluid of RA patients where it could act as factor to promote Th17 cell growth and differentiation. Unfortunately, the discovery of further genes/pathways induced by the combined blockade of IL-1β and IL-18 was hampered by the strong effect of the LPS/IL-12 stimulation of the PBMC samples. Nevertheless, both IFNγ and IL-26 and to some extend also IL-22 were also among the genes that were synergistically upregulated by the combined stimulation with recombinant IL-1β and IL-18 in PBMC, confirming that these two factors are downstream effectors in this activation pathway. Thus, the IL-20 subfamily of cytokines (including IL-26 and IL-22) seems to be strongly dependent on the simultaneous signals from IL-1β and IL-18. With all due caution about selectivity of the individual signalling signatures as well as potential efficacy of blocking, these comparisons are useful to show that the respective pathways are active in diseases like sarcoidosis.

### Example 6: Inhibition of Spontaneous IFNγ, TNFα and IL-2 Production by bbmAb1

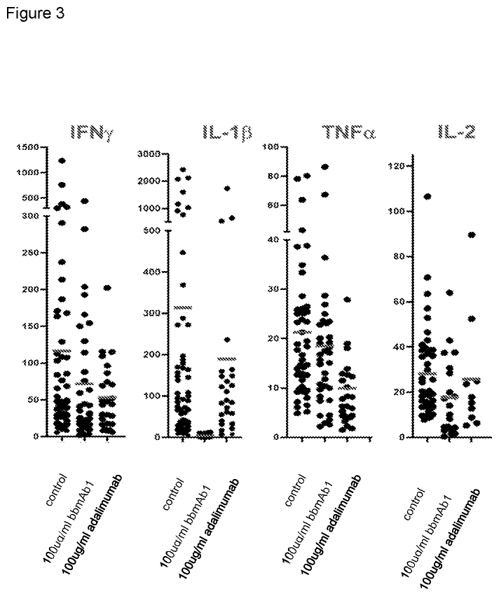

Punch biopsies (2 mm) were taken from surgically excised skin from 9 different HS patients and cultured in 80 μL of culture medium (Iscove's Modified Dulbecco's Medium supplemented with 10% KnockOut Serum Replacement (Gibco) and 1% Penicillin-Streptomycin) for 24 hours at 37° C. and 5% CO2 in 96 well cell culture plate (Flat bottom, Tissue Culture treated; Costar) either in culture media as untreated controls (FIG. 3, left most columns) or in the presence of either bbmAb1 (FIG. 3, middle columns) or Adalimumab (FIG. 3, right most columns) at a concentration of 100 μg/ml. After plate centrifugation, supernatants were collected from the individual HS biopsies and multiplex MSD (Meso Scale Discovery Platform) cytokine pro-inflammatory panel 1 (protein) array was performed according to the manufacturer's protocol using the MSD plate reader. Data were normalized with the individual biopsy weight and exported to GraphPad Prims software for blotting figures. FIG. 3 demonstrates that bbmAb1 reduces spontaneous IFNγ, TNFα and IL-2 production in supernatants of HS biopsies.

### Example 7: Toxicity Studies

The bispecific antibody bbmAb1 was well tolerated when administered to marmoset monkeys s.c. up to 100 mg/kg, twice weekly for 26 weeks (No Observed Effect Level (NOEL) 100 mg/kg; Cmax,ss of 3,110 μg/mL, AUC0-72h,ss of 218,000 μ·h/mL) and did not show any safety pharmacology (central nervous, cardiovascular and respiratory systems), toxicology (including the male reproductive system and sperm motility) or local tolerability effects. No effects were also seen after twice weekly intravenous (i.v.) dosing at 100 mg/kg for 26-weeks (Cmax,ss of 4570 μg/mL, AUC0-72h,ss of 261,000 μg·h/mL). Furthermore, no treatment-related effects were noted on immune cells in the peripheral blood as well as on primary and secondary humoral immune responses upon foreign antigen challenge. In a single ascending dose (SAD) study of bbmAb1, data on the first six cohorts (A1 to B1) with increasing doses of 0.1, 0.3, 1, 3, 10 mg/kg i.v. as well as 100 mg s.c in a total of 48 subjects (out of which 12 were placebo treated subjects), bbmAb1 was generally well tolerated in doses up to 10 mg/kg. The Cmax and AUC of bbmAb1 increased in a dose-proportional manner within the whole range of i.v. administration (0.1 mg/kg-10 mg/kg). The mean half-life of bbmAb1 was approximately 21 to 26 days. The bioavailability of a s.c. dose was estimated to be 70%.

### Example 8: Therapeutic Use

A Randomized, Subject and Investigator Blinded, Placebo-Controlled and Multi-Center Platform Study, to Assess Efficacy and Safety of bbmAb1 in Patients with Moderate to Severe Hidradenitis Suppurativa

Provided below are the details of the clinical trial design to demonstrate the efficacy of the bispecific anti-IL-1β anti-IL-18 antibody bbmAb1.

Blinding of subjects and investigators allows for an unbiased assessment of subjective readouts such as lesion counts in HS or global HS-PGA scores, as well as adverse events.

A randomized, subject and investigator blinded, placebo-controlled, multi-center and parallel-group study is run to assess efficacy, safety and tolerability of several active treatment compounds, such as bispecific anti-IL-1β anti-IL-18 antibody bbmAb1, in subjects with moderate to severe HS. After a screening period of approximately 5-weeks, the treatment period is planned for 16 weeks and is followed by a safety follow-up of approximately 12 weeks. Subjects are given bbmAb1, 300 mg (3 injections of 1.5 mL; bi-weekly on days 1, 15, and 29, then monthly (Q4W) on days 57 and 85) s.c. or its corresponding placebo (2×1.5 mL) s.c.

Subjects included in this study are adult male and female subjects of 18 to 65 years of age (inclusive), presenting with moderate to severe HS diagnosed with recurrent inflammatory lesions for at least 12 months. At randomization (pre-dose on Day 1), subjects need to have at least 5 inflammatory lesions (abscesses and nodules) in at least 2 anatomical areas to be included. Randomization to the cohorts and respective arms will be done using a centralized Interactive Response Technology (IRT) system.

The primary clinical endpoint is the simplified HiSCR (Hidradenitis Suppurativa Clinical Response) after 16 weeks of treatment.

On Day 113 (Week 17), after safety and other assessments have been performed, all subjects will enter the follow-up period and will not receive any further study drug administrations. If medically justified, and if no potential safety concerns have been identified (after discussion with the sponsor), subjects may receive previously prohibited medication during this follow-up period.

Safety and efficacy assessments will be conducted at follow-up visits as specified in the assessment schedule. Pharmacokinetic (PK), pharmacodynamic (PD), and biomarker samples will also be collected. The end of study (EOS) visit will occur on Day 197 (Week 29), and will include study completion evaluations followed by discharge from the study. Blinding will be maintained for the investigator and the subject until the study is completed.

Approximately 40 subjects are randomized; 30 subjects will receive the investigational treatment and 10 subjects will receive matching placebo.
On Day 1, 300 mg bbmAb1 or its corresponding placebo (2 injections of 1.5 mL) will be administered by subcutaneous injection (s.c.) by trained site personnel. Clinical assessments will be performed as well as PK, target engagement, immunogenicity, pathway disease and biomarkers and safety assessments. Subjects will be discharged from the site on the same day after completion of all assessments, provided there are no safety concerns. Following the first administration, subjects should be observed at the site for immediate injection site reactions for at least one hour, or longer at the discretion of the Investigator.
Subjects will return to the study center during the loading phase of the study (from Day 1 (Week 1) to Day 29 (Week 5)) to receive bbmAb1 every other week s.c. (Q2W; 3 doses).
Then during the maintenance phase of the study (from Day 29 (Week 5) to Day 85 (Week 13)) bbmAb1 will be administered at 300 mg s.c. every four weeks (Q4W; 2 doses).

Safety and selected efficacy assessments will be conducted during these visits and PK, target engagement, immunogenicity and pathway/disease biomarker samples will be collected.

The primary objective is to show preliminary efficacy of treatment with the bispecific antibody bbmAb1, in HS subjects after 16 weeks of treatment in comparison to placebo. After the 16-weeks treatment period a follow up period for 12 weeks is included to observe a sustainability of the effect can be sustained or increased after 16 weeks of treatment.

For this study, the simplified HiSCR (modified from Kimball 2014) was selected as the primary endpoint. The simplified HiSCR is defined as 50% decrease in the total number of abscesses plus inflammatory nodules, without any increase in draining fistulae.

The inflammatory lesions of HS will be counted as individual lesions (inflammatory nodules, abscesses and draining fistulae) in the typical anatomical areas. In addition to the count, a global assessment scale (Hidradenitis suppurativa-physician global assessment or HS-PGA) as well as a composite score (Severity Assessment of Hidradenitis suppurativa score or SAHS) will be used.

Several patient reported outcomes will be used, including the Dermatology Life Quality Index (DLQI). Finally, as from a subject's perspective skin related pain is the most important symptom, the numerical rating scale (NRS) for pain is included. Additional information on clinical assessments:

HS-PGA (Hidradenitis Suppurativa-Physician Global Assessment): The score will be used as exploratory objective to assess HS and was used and described in Kimball A B, Kerdel F, Adams D, et al (2012).

The SAHS score is a composite score (Hessam S, Scholl L, Sand M, et al (2018)) and will be derived from the collected information for inflammatory lesion count, the fistulae count, and the NRS pain. In addition, the anatomical areas and the new or flared existing boils will be collected in both cohorts.

Skin Pain-NRS (numerical rating scale for pain): An NRS for skin related pain was used in adalimumab studies (Kimball et al. (2016)) and will be used as skin or HS related pain is one of the highest burden for the patient (Matusiak et al (2017)). The pain that is HS related will be recorded on average in the last 24 hours and at worst (in the last 24 hours).

Other Patient reported outcomes (PRO) will include aspects as itching, fatigue and work impairment, as well as Dermatology Life Quality Index (DLQI) and dermatology related Quality of life (QoL) tool with validated scores available in many countries and languages. It includes a Patient Global Assessment.

Objectives and Related Endpoints

| Primary objective(s) | Endpoint(s) for primary objective(s) |
|---|---|
| To assess the efficacy of the | Proportion of patients achieving clinical |
| investigational treatments, | response evaluated by the simplified |
| compared to placebo in moderate to | Hidradenitis Suppurativa Clinical |
| severe inflammatory hidradenitis | Response (HiSCR) after 16 weeks of |
| suppurativa (HS) patients | treatment |
| Secondary objective(s) | Endpoint(s) for secondary objective(s) |
| To assess the safety and tolerability | Number and severity of AEs |
| of the investigational treatments in | Physical examination, vital signs, safety |
| patients with moderate to severe | laboratory measurements, ECGs at |
| hidradenitis suppurativa (HS) | baseline and repeatedly until study |
| | completion visit |
| To explore the effect of the | Hidradenitis Suppurativa - Physician's |
| investigational treatments versus | Global Assessment (HS-PGA) |
| placebo on other efficacy | responders over time |
| measurements over time | HS lesion count over time |
| | SAHS Score |
| | International Hidradenitis Suppurativa |
| | Score System (IHS4) |
| To assess the effect of the | Dermatology Life Quality Index (DLQI) |
| investigational treatments compared | Patient's Global Assessment (PGA) |
| to placebo on patient reported | At selected sites only: Hidradenitis |
| outcomes (PRO) | Suppurativa Symptom Diary (HSSD) |
| | Proportion of patients achieving NRS30 |
| | after 16 weeks of treatment, among |
| | patients with baseline Skin Pain NRS ≥3 |
| | using the Numerical Rating Scale of Pain |
| | Assessment (NRS) |
| | Number of new boils or existing boils |
| | which flare up in the past four weeks |
| To explore the potential of the | Proportion of patients who experience at |
| investigational treatments to reduce | least one flare over 16 weeks of treatment |
| HS flares versus placebo | |
| To assess clinical activity of the | Proportion of patients achieving clinical |
| investigational treatments in | response evaluated by the HiSCR and |
| moderate to severe inflammatory HS | simplified HiSCR at each visit |
| patients over time | |

### Key Inclusion Criteria:

Male and female subjects, 18 to 65 years of age (inclusive), with clinically diagnosed HS for at least 12 months prior to screening
Patients with moderate to severe HS, as per evaluation at screening and randomization (pre-dose on Day 1):
   A total of at least 5 inflammatory lesions, i.e., abscesses and/or inflammatory nodules, and
   No more than 15 fistulae, and
   At least two anatomical areas need to be involved with HS lesions
Minimal body weight of 50 kg (inclusive) at screening

Able to communicate well with the investigator and understand and comply with the requirements of the study, and the ability and willingness to conduct study visits as per the study schedule.

### Key Exclusion Criteria:

Use of other investigational drugs at the time of screening, or within 30 days or 5 half-lives of randomization, whichever is longer; or longer if required by local regulations.
WOCBP (defined as all women physiologically capable of becoming pregnant) will be asked to adhere to highly effective contraception from at least 3 months prior to first drug administration and until 5 months after the final dose (Day 225 to Day 253), when a pregnancy test will be conducted.
Pregnant or nursing (lactating) women at screening or randomization, where pregnancy is defined as the state of a female after conception and until the termination of gestation, confirmed by a positive hCG laboratory test.

### Study Treatment & Duration

Patients assigned to the bbmAb1 arm will receive bbmAb1, 300 mg, s.c. or matching placebo as follows: Bi-weekly (Q2W) from Day 1 (week 1) to Day 29 (Week 5) and then monthly (Q4W) until Day 85 included (Week 13)

### Efficacy Assessments

Simplified and original Hidradenitis suppurativa clinical response (HiSCR) rate
International Hidradenitis Suppurativa Severity Score System (IHS4)
Hidradenitis suppurativa-Physician Global Assessment (HS-PGA) score and responder rate
HS inflammatory lesion count
Severity Assessment of Hidradenitis Suppurativa (SAHS)

For bbmAb1, the dosage form of the supplied drug is a “ready to use” aqueous buffered sterile solution. The solution contains 100 mg/ml bbmAb1 and the excipients L-histidine, sucrose, and polysorbate 20, pH 6.0. The placebo control, selected for this study, is a solution with a matching composition of inactive excipients.

Predicted Effect of bbmAb1 Doses on IL-1β and Free IL-18

The model used to predict the dynamics of the anti-IL-1β/IL-18 bispecific antibody and its targets in serum consists of a general competitive binding model for the IL-18 arm (Yan et al 2012) and the previously published model of canakinumab adjusted to bbmAb1 (Chakraborty et al 2012) for the IL-1β arm with a novel model describing the free and total IL-18 dynamics. To predict the dynamics of IL-1β in response to the application of bbmAb1, the model established in the clinical canakinumab study was used (Chakraborty et al 2012) and the bbmAb1-specific PK parameters and binding affinities were updated accordingly. To adjust the IL-1β concentrations in CAPS patients, we used the synthesis and clearance parameters of this interleukin listed in the canakinumab clinical study (Chakraborty et al 2012). The model is based on in-house in vitro and published human data from free and total IL-18 serum concentrations from patients across several autoimmune diseases (Weiss et al 2018).

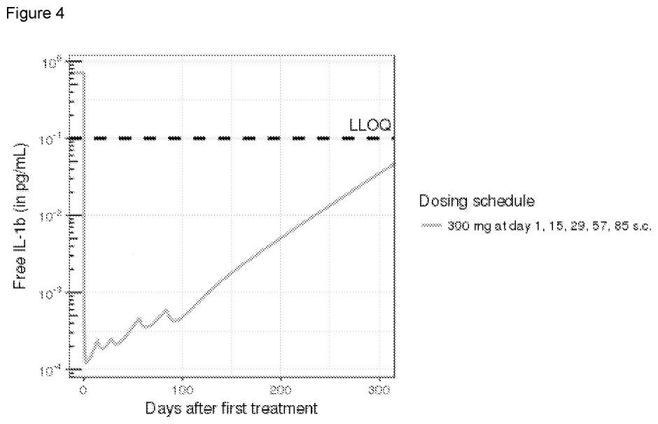

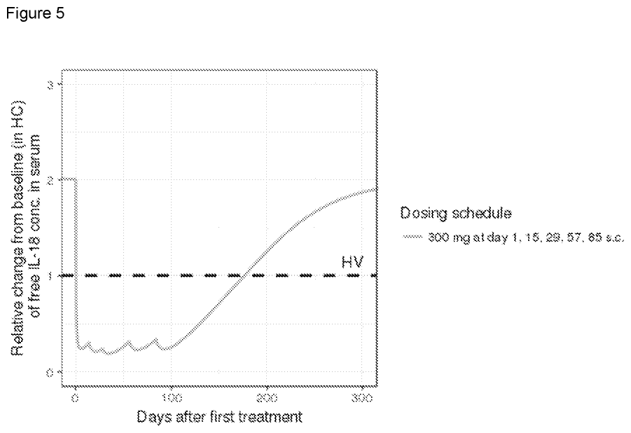

Based on this, a dosing schedule of 300 mg Q2W/Q4W s.c. is predicted to result in the simultaneous reduction of both IL-1β and IL-18 levels in serum and effective neutralization of IL-1β as well as IL-18 is expected (FIG. 4 and FIG. 5).

Pharmacokinetics of bbmAb1

bbmAb1 is evaluated in a FiH single dose ascending study up to 10 mg/kg i.v. in healthy volunteers without any drug related SAEs. The pharmacokinetics (PK) of bbmAb1 in human follows human prediction based on marmoset monkey data and is as expected for a typical IgG1 antibody binding to soluble ligand cytokine target(s). bbmAb1 showed a dose linear increase in exposure matching the predicted human PK (evaluated up to 10 mg/kg i.v.). The predicted human PK parameters of bbmAb1 are: clearance (CL)=0.158 L/d, volume of distribution (V_{d})=5.586 L (for a 70-kg human subject) or 0.08 L/kg; half-life (T_{1/2})=24.5 days. Preliminary analysis of PK profiles from the FiH study has not provided evidence of accelerated clearance of bbmAb1 due to the formation of anti-drug antibodies (ADA).

Based on recent subcutaneous data from the FiH study, bioavailability and absorption rate constants were adjusted due to these findings and used in the prediction of the subcutaneous PK.

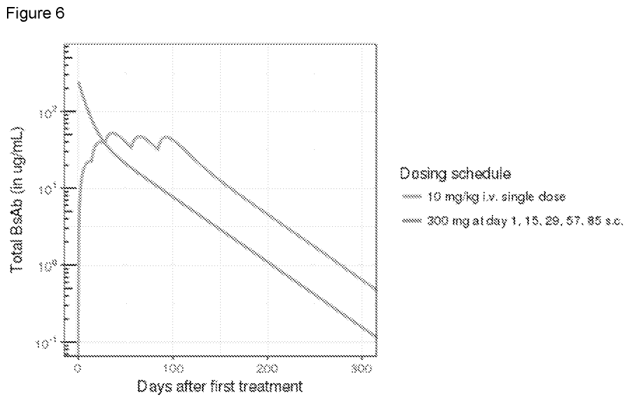

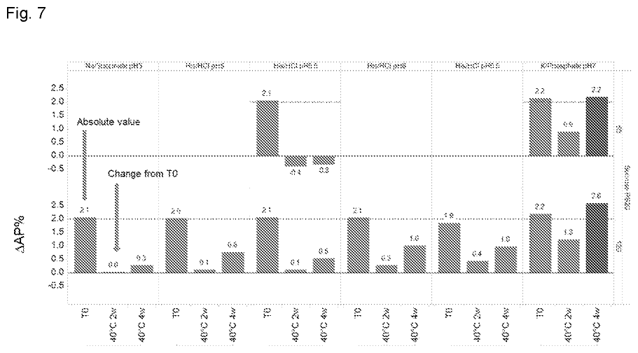

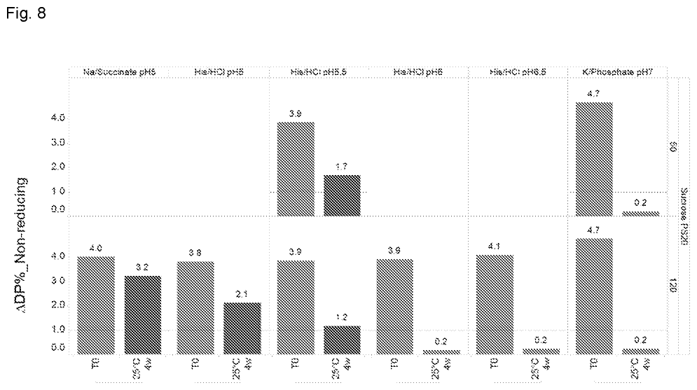

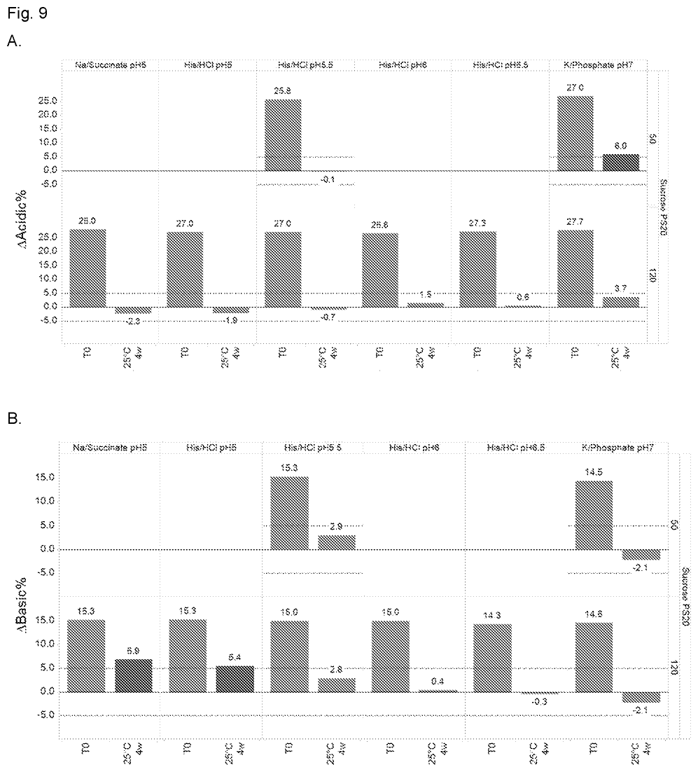

The bbmAb1 dose of 300 mg Q2W/Q4W s.c. is predicted to lead to rapid and simultaneous neutralization of all systemic free IL-1β and IL-18. The exposure after 300 mg s.c. will exceed the in vitro IC90 for IL-1β and IL-18 for more than 100 days after a single dose (FIG. 6).

### REFERENCES

Akdogan N, Dogan S, Incel-Uysal P, et al (2020) Serum amyloid A and C-reactive protein levels and erythrocyte sedimentation rate are important indicators in hidradenitis suppurativa. Arch Dermatol Res.; 312(4):255-262.
Andersen R K, Jemec G B (2017) Treatments for hidradenitis suppurativa. Clin. Dermatol. p. 218-224.
André R, Marescassier H, Gabay C, et al (2019) Long-term therapy with anakinra in hidradenitis suppurativa in three patients. Int J Dermatol; 58(11):e208-e209.
Bettoli V, Zauli S, Virgili A (2016) Oral clindamycin and rifampicin in the treatment of hidradenitis suppurativa-acne inversa: can some factors influence the response to the treatment? G Ital Dermatol Venereol p. 216-8.
Blok J L, Li K, Brodmerkel C, et al (2016) Ustekinumab in hidradenitis suppurativa: clinical results and a search for potential biomarkers in serum. Br. J. Dermatol. p. 839-46.
Byrd A S, Kerns M L, Williams D W, et al (2018) Collagen deposition in chronic hidradenitis suppurativa: potential role for CD163+macrophages. Br. J. Dermatol.; 179(3):792-794.
Börgeson E, Docherty N G, Murphy M, et al (2011) Lipoxin A4 and benzo-lipoxin A4 attenuate experimental renal fibrosis. FASEB J. p. 2967-79.
Casseres R G, Prussick L, Zancanaro P, et al (2020) Secukinumab in the treatment of moderate to severe hidradenitis suppurativa: Results of an open-label trial. J Am Acad Dermatol.; 82(6): 1524-1526
Celtaxsys Inc. Press-release (2018) Celtaxsys announces results of phase 2 trial showing clinically meaningful improvement in pulmonary exacerbations in cystic fibrosis patients.
Chakraborty, A, Tannenbaum, S, Rordorf, C. et al (2012) Pharmacokinetic and pharmacodynamic properties of canakinumab, a human anti-interleukin-1β monoclonal antibody. Clinical pharmacokinetics; 51(6):e1-e18.
Christmas P, Fox J W, Ursino S R, et al (1999) Differential localization of 5-and 15-lipoxygenases to the nuclear envelope in RAW macrophages. J. Biol. Chem. p. 25594-8.
Deckers I E, Kimball A B (2016) The Handicap of Hidradenitis Suppurativa. Dermatol Clin p. 17-22.
Dessinioti C, Zisimou C, Tzanetakou V, et al (2016) Oral clindamycin and rifampicin combination therapy for hidradenitis suppurativa: a prospective study and 1-year follow-up. Clin. Exp. Dermatol. p. 852-857.
Esmann S, Jemec G B (2011) Psychosocial impact of hidradenitis suppurativa: a qualitative study. Acta Derm. Venereol. p. 328-32.
Fimmel S, Zouboulis C C (2010) Comorbidities of hidradenitis suppurativa (acne inversa). Dermatoendocrinol; 2(1):9-16 . . .
Finlay A Y, Khan G K (1994) Dermatology Life Quality Index (DLQI)—a simple practical measure for routine clinical use. Clin. Exp. Dermatol; 19: 210-216.
Garg A, Wertenteil S, Baltz R, et al (2018) Prevalence Estimates for Hidradenitis Suppurativa among Children and Adolescents in the United States: A Gender- and Age-Adjusted Population Analysis. J. Invest. Dermatol.; 138(10):2152-2156.
Giamarellos-Bourboulis E J, Argyropoulou M, Kanni T, et al (2020). Clinical efficacy of complement C5a inhibition by IFX-1 in hidradenitis suppurativa: an open-label single-arm trial in patients not eligible for adalimumab. Br J Dermatol.; 183(1):176-178.
Giuseppe P, Nicola P, Valentina C, et al (2018) A Case of Moderate Hidradenitis Suppurativa and Psoriasis Treated with Secukinumab. Ann Dermatol; 30(4):462-464.
Gottlieb A, Natsis N E, Kerdel F, et al (2020) A Phase I I Open-Label Study of Bermekimab in Patients with Hidradenitis Suppurativa Shows Resolution of Inflammatory Lesions and Pain. J Invest Dermatol; 140(8): 1538-1545.
Hessam S, Scholl L, Sand M, et al (2018) A Novel Severity Assessment Scoring System for Hidradenitis Suppurativa. JAMA Dermatol; 154(3):330-335.
Hessam S, Sand M, Gambichler T, et al (2015) Correlation of inflammatory serum markers with disease severity in patients with hidradenitis suppurativa (H S). J Am Acad Dermatol.; 73(6):998-1005.
Houriet C, Seyed Jafari S M, Thomi R, et al (2017) Canakinumab for Severe Hidradenitis Suppurativa: Preliminary Experience in 2 Cases. JAMA Dermatol.; 1;153(11):1195-1197.
Hunger R E, Surovy A M, Hassan A S, et al (2008) Toll-like receptor 2 is highly expressed in lesions of acne inversa and colocalizes with C-type lectin receptor. Br. J. Dermatol.; 158(4):691-7.
Ingram J R, Woo P N, Chua S L, et al (2016) Interventions for hidradenitis suppurativa: a Cochrane systematic review incorporating GRADE assessment of evidence quality. Br. J. Dermatol.; 174(5):. 970-8.
International Council for Harmonization of Technical Requirements for Pharmaceuticals for Human Use (ICH) (2016) ICH E6 (R2) GCP Guideline (2016).
Jaeger T, Andres C, Grosber M, et al (2013) Pyoderma gangrenosum and concomitant hidradenitis suppurativa-rapid response to canakinumab (anti-IL-1β). Eur J Dermatol.; 23(3):408-410.
Janse I C, Deckers I E, van der Maten A D, et al (2017) Sexual health and quality of life are impaired in hidradenitis suppurativa: a multicentre cross-sectional study. Br. J. Dermatol.; 176(4):1042-1047.
Jemec G B (2012) Clinical practice. Hidradenitis suppurativa. N. Engl. J. Med.; 366(2): 158-64.
Jemec G B, Kimball A B (2015) Hidradenitis suppurativa: Epidemiology and scope of the problem. J. Am. Acad. Dermatol.; 73(5 Suppl 1):S4-7.
Jørgensen A R, Yao Y, Thomsen S F (2018) Therapeutic Response to Secukinumab in a 36-Year-Old Woman with Hidradenitis Suppurativa. Case Rep Dermatol Med; (April 16):8685136 . . .
Kanni T, Zenker O, Habel M, et al (2018) Complement activation in hidradenitis suppurativa: a new pathway of pathogenesis? Br. J. Dermatol.; 179(2):413-419
Kelly G, Sweeney C M, Tobin A M, et al (2014) Hidradenitis suppurativa: the role of immune dysregulation. Int. J. Dermatol.; 53(10):1186-96.
Kelly G, Hughes R, McGarry T, et al (2015) Dysregulated cytokine expression in lesional and nonlesional skin in hidradenitis suppurativa. Br J Dermatol.; 173(6):1431-1439.
Kimball A B, Ganguli A, Fleischer A (2018) Reliability of the hidradenitis suppurativa clinical response in the assessment of patients with hidradenitis suppurativa. J Eur Acad Dermatol Venereol.; 32(12):2254-2256.
Kimball A B, Jemec G B, Yang M, et al (2014) Assessing the validity, responsiveness and meaningfulness of the Hidradenitis Suppurativa Clinical Response (HiSCR) as the clinical endpoint for hidradenitis suppurativa treatment. Br. J. Dermatol.; 171(6): 1434-42.
Kimball A B, Kerdel F, Adams D, et al (2012) Adalimumab for the treatment of moderate to severe Hidradenitis suppurativa: a parallel randomized trial. Ann. Intern. Med.; 157:846-855.
Kimball A B, Okun M M, Williams D A, et al (2016) Two Phase 3 Trials of Adalimumab for Hidradenitis Suppurativa. N. Engl. J. Med.; 375(5): 422-34.
Leslie K S, Tripathi S V, Nguyen T V, et al (2014) An open-label study of anakinra for the treatment of moderate to severe hidradenitis suppurativa. J Am Acad Dermatol.; 70(2):243-251.
Lima A L, Karl I, Giner T, et al (2016) Keratinocytes and neutrophils are important sources of proinflammatory molecules in hidradenitis suppurativa. Br. J. Dermatol.; 174(3): 514-21.
Ljungman P, Boeckh M, Hirsch H H, et al (2017) Disease Definitions Working Group of the Cytomegalovirus Drug Development Forum. Definitions of Cytomegalovirus Infection and Disease in Transplant Patients for Use in Clinical Trials. Clin Infect Dis.; 64(1):87-91.
Musilova J, Moran B, Sweeney C M, et al (2020) Enrichment of Plasma Cells in the Peripheral Blood and Skin of Patients with Hidradenitis Suppurativa. J Invest Dermatol; 140(5): 1091-1094 Penno C A, Jäger P, Laguerre C et al (2020) Lipidomics Profiling of Hidradenitis Suppurativa Skin Lesions Reveals Lipoxygenase Pathway Dysregulation and Accumulation of Pro-Inflammatory Leukotriene B4. J Invest Dermatol.; 140(12): 2421-2432 . . .
Reguiaï Z, Fougerousse A C, Maccari F, et al (2020) Effectiveness of secukinumab in hidradenitis suppurativa: an open study (20 cases). J Eur Acad Dermatol Venereol.; 34(11):e750-e751.
Revuz J (2009) Hidradenitis suppurativa. J European Acad Dermatol Venereol; 23:985 998.
Riis P T, Søeby K, Saunte D M, et al (2015) Patients with hidradenitis suppurativa carry a higher systemic inflammatory load than other dermatological patients. Arch Dermatol Res.; 307(10):885-9.
Russo V, Alikhan A (2016). Failure of Anakinra in a Case of Severe Hidradenitis Suppurativa. J Drugs Dermatol.; 15(6):772-774.
Schuch A, Fischer T, Boehner A, et al (2018) Successful Treatment of Severe Recalcitrant Hidradenitis Suppurativa with the Interleukin-17A Antibody Secukinumab. Acta Derm. Venereol.; 98(1): 151-152.
Serhan C N, Chiang N, Van Dyke T E (2008) Resolving inflammation: dual anti-inflammatory and pro-resolution lipid mediators. Nat. Rev. Immunol.; 8(5): 349-61.
Shah A, Alhusayen R, Amini-Nik S (2017) The critical role of macrophages in the pathogenesis of hidradenitis suppurativa. Inflamm. Res.; 66(11): 931-945.
Sharon V R, Garcia M S, Bagheri S, et al (2012) Management of recalcitrant hidradenitis suppurativa with ustekinumab. Acta Derm. Venereol.; 92(3):320-1.
Singer E A (2004) The necessity and the value of placebo. Sci Eng Ethics, 10(1): 51-6.
Sun N Z, Ro T, Jolly P, et al (2017) Non-response to Interleukin-1 Antagonist Canakinumab in Two Patients with Refractory Pyoderma Gangrenosum and Hidradenitis Suppurativa. J Clin Aesthet Dermatol.; 10(9):36-38.
Tekin B, Salman A, Ergun T (2017) Hidradenitis suppurativa unresponsive to canakinumab treatment: A case report. Indian J Dermatol Venereol Leprol.; 83(5):615-617.
Thorlacius L, Theut Riis P, Jemec G B E (2018) Severe hidradenitis suppurativa responding to treatment with secukinumab: a case report. Br. J. Dermatol.; 179(1): 182-185.
Tzanetakou V, Kanni T, Giatrakou S, et al (2016) Safety and Efficacy of Anakinra in Severe Hidradenitis Suppurativa: A Randomized Clinical Trial. JAMA Dermatol; 152(1): 52-59.
Van der Zee H H, Laman J D, de Ruiter L, et al (2012) Adalimumab (antitumour necrosis factor-α) treatment of hidradenitis suppurativa ameliorates skin inflammation: an in situ and ex vivo study. Br. J. Dermatol; 166(2):298-305.
van der Zee H H, Prens E P (2013) Failure of anti-interleukin-1 therapy in severe hidradenitis suppurativa: a case report. Dermatology; 226(2):97-100. Weber P, Seyed Jafari S M, Yawalkar N, et al (2017) Apremilast in the treatment of moderate to severe hidradenitis suppurativa: A case series of 9 patients. J. Am. Acad. Dermatol.; 76(6): 1189-1191.
Weiss, E S, Girard-Guyonvarc′h, C, Holzinger, D, et al (2018) Interleukin-18 diagnostically distinguishes and pathogenically promotes human and murine macrophage activation syndrome. Blood, The Journal of the American Society of Hematology; 131(13):1442-1455.8
Witte-Händel E, Wolk K, Tsaousi A, et al (2019) The IL-1 Pathway Is Hyperactive in Hidradenitis Suppurativa and Contributes to Skin Infiltration and Destruction. J Invest Dermatol.; 139(6):1294-1305.
Woodcock J, LaVange L M (2017) Master Protocols to Study Multiple Therapies, Multiple Diseases, or Both. N. Engl. J. Med.; 377: 62-70.
Yan, X, Chen, Y and Krzyzanski, W (2012). Methods of solving rapid binding target-mediated drug disposition model for two drugs competing for the same receptor. Journal of pharmacokinetics and pharmacodynamics; 39(5): 543-560.
Zarchi K, Dufour D N, Jemec G B (2013) Successful treatment of severe hidradenitis suppurativa with anakinra. JAMA Dermatol.; 149(10):1192-1194.
Zouboulis C C (2009) Zileuton, a new efficient and safe systemic anti-acne drug. Dermatoendocrinol; 1(3):188-92.
Zouboulis C C, Desai N, Emtestam L, et al (2015) European S1 guideline for the treatment of hidradenitis suppurativa/acne inversa. J Eur Acad Dermatol Venereol; 29(4): 619-44.
Zouboulis C C, Nestoris S, Adler Y D, et al (2003) A new concept for acne therapy: a pilot study with zileuton, an oral 5-lipoxygenase inhibitor. Arch Dermatol; 139(5): 668-70.
Zouboulis C C, Tzellos T, Kyrgidis A, et al on behalf of European Hidradenitis Suppurativa Foundation Investigator Group (2017). Development and validation of the International Hidradenitis Suppurativa Severity Score System (IHS4), a novel dynamic scoring system to assess H S severity. Br. J. Dermatol.; 177(5): 1401-9.

### Example 9: Development of a High Concentration Formulation (100 to 120 mg/mL) of bbmAb1

Eight different formulations were initially tested in high-throughput plate-based assays.
F1 (120 mg/mL bbmAb1, 20 mM Sodium Succinate, pH 5.0, 220 mM Sucrose, 0.04% polysorbate 20);
F2 (120 mg/mL bbmAb1, 20 mM Histidine/Histidine-Cl, pH 5.0, 220 mM Sucrose, 0.04% polysorbate 20);
F3 (120 mg/mL bbmAb1, 20 mM Histidine/Histidine-Cl, pH 5.5, 220 mM Sucrose, 0.04% polysorbate 20);
F4 (120 mg/mL bbmAb1, 20 mM Histidine/Histidine-Cl, pH 6.0, 220 mM Sucrose, 0.04% polysorbate 20);
F5 (120 mg/mL bbmAb1, 20 mM Histidine/Histidine-Cl, pH 6.5, 220 mM Sucrose, 0.04% polysorbate 20);
F6 (120 mg/mL bbmAb1, 20 mM Potassium Phosphate, pH 7.0, 220 mM Sucrose, 0.04% polysorbate 20);
F7 (50 mg/mL bbmAb1, 20 mM Histidine/Histidine-Cl, PH 5.5, 220 mM Sucrose, 0.04% polysorbate 20);
F8 (50 mg/mL bbmAb1, 20 mM Potassium Phosphate, pH 7.0, 220 mM Sucrose, 0.04% polysorbate 20);

Formulations F1 to F8 were tested for aggregate formation by size exclusion chromatography after 2 weeks or 4 weeks at 40° C. The results showed a trend of increased aggregation as pH was increased from 5 to 7, with the pH 7.0 formulations F6 and F8 exhibiting significant aggregation. See, FIG. 7.

Formulations F1 to F8 were also tested for degradation product formation by size exclusion chromatography. The results indicated that the protein was stable in all formulations tested under the test conditions (25° C., 4 weeks). However, LabChip analysis under non-reducing conditions showed a clear trend of degradation product formation at lower pH, with pH 5.0 formulations exhibiting significant degradation product formation. See, FIG. 8.

Formulations F1 to F8 were tested for acidic and basic variant formation in response to thermal stress (25° C., 4W) by charge zone electrophoresis (CZE). Significant acidic variant formation was observed at high pH (F8) and significant basic variant formation was observed at low pH (F1 and F2). See, FIGS. 9A and 9B.

The above formulations were also tested for resistance to freeze thaw, and mechanical stress (agitation), and the results further support successful stable formulation at pH 5.5, preferably 6.0, with 50 to 120 mg/mL bbmAb1 (preferably 100 mg/mL bbmAb1) with an excipient (e.g., a sugar such as sucrose) and surfactant (e.g., a polysorbate, such as polysorbate 20).

Additional testing modalities included visual, turbidity (A405 nm), dynamic light scattering, microflow imaging, and LysC peptide mapping by liquid chromatography mass spectrometry.

### Sequence Table

Useful amino acid and nucleotide sequences for practicing the invention are disclosed in Table 15.

TABLE 15
Sequences according to embodiments of the invention

| SEQ ID NUMBER | Ab region | Sequence |
|---|---|---|
| mAb1 | | |
| SEQ ID NO: 1 (Kabat) | HCDR1 | SYAIS |
| SEQ ID NO: 2 (Kabat) | HCDR2 | NIIPMTGQTYYAQKFQG |
| SEQ ID NO: 3 (Kabat) | HCDR3 | AAYHPLVFDN |
| SEQ ID NO: 4 (Chothia) | HCDR1 | GGTFKSY |
| SEQ ID NO: 5 (Chothia) | HCDR2 | IPMTGQ |
| SEQ ID NO: 6 (Chothia) | HCDR3 | AAYHPLVFDN |
| SEQ ID NO: 7 | VH | EVQLVQSGAEVKKPGSSVKVSCKASG |
| | | GTFKSYAISWVRQAPGQGLEWMGNIIP |
| | | MTGQTYYAQKFQGRVTITADESTSTAY |
| | | MELSSLRSEDTAVYYCARAAYHPLVFD |
| | | NWGQGTLVTVSS |
| SEQ ID NO: 8 | DNA VH | GAGGTGCAGCTGGTGCAGAGCGGCG |
| | | CCGAGGTGAAGAAGCCCGGCAGCAG |
| | | CGTGAAGGTGAGCTGCAAGGCCAGC |
| | | GGCGGCACCTTCAAGAGCTACGCCA |
| | | TCAGCTGGGTGAGGCAGGCCCCCGG |
| | | CCAGGGCCTGGAGTGGATGGGCAAC |
| | | ATCATCCCCATGACCGGCCAGACCTA |
| | | CTACGCCCAGAAGTTCCAGGGCAGG |
| | | GTGACCATCACCGCCGACGAGAGCA |
| | | CCAGCACCGCCTACATGGAGCTGAG |
| | | CAGCCTGAGGAGCGAGGACACCGCC |
| | | GTGTACTACTGCGCCAGGGCCGCCT |
| | | ACCACCCCCTGGTGTTCGACAACTG |
| | | GGCCAGGGCACCCTGGTGACCGTGA |
| | | GCAGC |
| SEQ ID NO: 9 | Heavy Chain | EVQLVQSGAEVKKPGSSVKVSCKASG |
| | | GTFKSYAISWVRQAPGQGLEWMGNIIP |
| | | MTGQTYYAQKFQGRVTITADESTSTAY |
| | | MELSSLRSEDTAVYYCARAAYHPLVFD |
| | | NWGQGTLVTVSSASTKGPSVFPLAPS |
| | | SKSTSGGTAALGCLVKDYFPEPVTVS |
| | | WNSGALTSGVHTFPAVLQSSGLYSLSS |
| | | VVTVPSSSLGTQTYICNVNHKPSNTKV |
| | | DKRVEPKSCDKTHTCPPCPAPEAAGG |
| | | PSVFLFPPKPKDTLMISRTPEVTCVVVD |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSREEMTKNQVSLTCLVKGF |
| | | YPSDIAVEWESNGQPENNYKTTPPVLD |
| | | SDGSFFLYSKLTVDKSRWQQGNVFSC |
| | | SVMHEALHNHYTQKSLSLSPGK |
| SEQ ID NO: 10 | DNA Heavy Chain | GAGGTGCAGCTGGTGCAGAGCGGCGCCGAGG |
| | | TGAAGAAGCCCGGCAGCAGCGTGAAGGTG |
| | | AGCTGCAAGGCCAGCGGCGGCACCTTCAAGA |
| | | GCTACGCCATCAGCTGGGTGAGGCAGGCC |
| | | CCCGGCCAGGGCCTGGAGTGGATGGGCAACA |
| | | TCATCCCCATGACCGGCCAGACCTACTAC |
| | | GCCCAGAAGTTCCAGGGCAGGGTGACCATCAC |
| | | CGCCGACGAGAGCACCAGCACCGCCTAC |
| | | ATGGAGCTGAGCAGCCTGAGGAGCGAGGACA |
| | | CCGCCGTGTACTACTGCGCCAGGGCCGCC |
| | | TACCACCCCCTGGTGTTCGACAACTGGGGCCA |
| | | GGGCACCCTGGTGACCGTGAGCAGCGCC |
| | | AGCACCAAGGGCCCCAGCGTGTTCCCCCTGGC |
| | | CCCCAGCAGCAAGAGCACCAGCGGCGGC |
| | | ACCGCCGCCCTGGGCTGCCTGGTGAAGGACTA |
| | | CTTCCCCGAGCCCGTGACCGTGAGCTGG |
| | | AACAGCGGCGCCCTGACCAGCGGCGTGCACA |
| | | CCTTCCCCGCCGTGCTGCAGAGCAGCGGC |
| | | CTGTACAGCCTGAGCAGCGTGGTGACCGTGCC |
| | | CAGCAGCAGCCTGGGCACCCAGACCTAC |
| | | ATCTGCAACGTGAACCACAAGCCCAGCAACAC |
| | | CAAGGTGGACAAGAGGGTGGAGCCCAAG |
| | | AGCTGCGACAAGACCCACACCTGCCCCCCCTG |
| | | CCCCGCCCCCGAGGCCGCCGGCGGCCCC |
| | | AGCGTGTTCCTGTTCCCCCCCAAGCCCAAGGA |
| | | CACCCTGATGATCAGCAGGACCCCCGAG |
| | | GTGACCTGCGTGGTGGTGGACGTGAGCCACG |
| | | AGGACCCCGAGGTGAAGTTCAACTGGTAC |
| | | GTGGACGGCGTGGAGGTGCACAACGCCAAGA |
| | | CCAAGCCCAGGGAGGAGCAGTACAACAGC |
| | | ACCTACAGGGTGGTGAGCGTGCTGACCGTGCT |
| | | GCACCAGGACTGGCTGAACGGCAAGGAG |
| | | TACAAGTGCAAGGTGAGCAACAAGGCCCTGCC |
| | | CGCCCCCATCGAGAAGACCATCAGCAAG |
| | | GCCAAGGGCCAGCCCAGGGAGCCCCAGGTGT |
| | | ACACCCTGCCCCCCAGCAGGGAGGAGATG |
| | | ACCAAGAACCAGGTGAGCCTGACCTGCCTGGT |
| | | GAAGGGCTTCTACCCCAGCGACATCGCC |
| | | GTGGAGTGGGAGAGCAACGGCCAGCCCGAGA |
| | | ACAACTACAAGACCACCCCCCCCGTGCTG |
| | | GACAGCGACGGCAGCTTCTTCCTGTACAGCAA |
| | | GCTGACCGTGGACAAGAGCAGGTGGCAG |
| | | CAGGGCAACGTGTTCAGCTGCAGCGTGATGCA |
| | | CGAGGCCCTGCACAACCACTACACCCAG |
| | | AAGAGCCTGAGCCTGAGCCCCGGCAAG |
| SEQ ID NO: 11 (Kabat) | LCDR1 | SGSSSNIGNHYVN |
| SEQ ID NO: 12 (Kabat) | LCDR2 | RNNHRPS |
| SEQ ID NO: 13 (Kabat) | LCDR3 | QSWDYSGFSTV |
| SEQ ID NO: 14 (Chothia) | LCDR1 | SSSNIGNHY |
| SEQ ID NO: 15 (Chothia) | LCDR2 | RNN |
| SEQ ID NO: 16 (Chothia) | LCDR3 | WDYSGFST |
| SEQ ID NO: 17 | VL | DIVLTQPPSVSGAPGQRVTISCSGSSS |
| | | NIGNHYVNWYQQLPGTAPKLLIYRNNH |
| | | RPSGVPDRFSGSKSGTSASLAITGLQS |
| | | EDEADYYCQSWDYSGFSTVFGGGTKL |
| | | TVL |
| SEQ ID NO: 18 | DNA VL | GATATCGTCCTGACTCAGCCCCCTAG |
| | | CGTCAGCGGCGCTCCCGGTCAGAGA |
| | | GTGACTATTAGCTGTAGCGGCTCTAG |
| | | CTCTAATATCGGTAATCACTACGTGA |
| | | ACTGGTATCAGCAGCTGCCCGGCAC |
| | | CGCCCCTAAGCTGCTGATCTATAGAA |
| | | ACAATCACCGGCCTAGCGGCGTGCC |
| | | CGATAGGTTTAGCGGATCTAAGTCAG |
| | | GCACTAGCGCTAGTCTGGCTATCACC |
| | | GGACTGCAGTCAGAGGACGAGGCCG |
| | | ACTACTACTGTCAGTCCTGGGACTAT |
| | | AGCGGCTTTAGCACCGTGTTCGGCG |
| | | GAGGCACTAAGCTGACCGTGCTG |
| SEQ ID NO: 19 | Light Chain | DIVLTQPPSVSGAPGQRVTISCSGSSS |
| | | NIGNHYVNWYQQLPGTAPKLLIYRNNH |
| | | RPSGVPDRFSGSKSGTSASLAITGLQS |
| | | EDEADYYCQSWDYSGFSTVFGGGTKL |
| | | TVLGQPKAAPSVTLFPPSSEELQANKA |
| | | TLVCLISDFYPGAVTVAWKADSSPVKA |
| | | GVETTTPSKQSNNKYAASSYLSLTPEQ |
| | | WKSHRSYSCQVTHEGSTVEKTVAPTE |
| | | CS |
| SEQ ID NO: 20 | DNA Light Chain | GATATCGTCCTGACTCAGCCCCCTAG |
| | | CGTCAGCGGCGCTCCCGGTCAGAGA |
| | | GTGACTATTAGCTGTAGCGGCTCTAG |
| | | CTCTAATATCGGTAATCACTACGTGA |
| | | ACTGGTATCAGCAGCTGCCCGGCAC |
| | | CGCCCCTAAGCTGCTGATCTATAGAA |
| | | ACAATCACCGGCCTAGCGGCGTGCC |
| | | CGATAGGTTTAGCGGATCTAAGTCAG |
| | | GCACTAGCGCTAGTCTGGCTATCACC |
| | | GGACTGCAGTCAGAGGACGAGGCCG |
| | | ACTACTACTGTCAGTCCTGGGACTAT |
| | | AGCGGCTTTAGCACCGTGTTCGGCG |
| | | GAGGCACTAAGCTGACCGTGCTGGG |
| | | TCAGCCTAAGGCTGCCCCCAGCGTG |
| | | ACCCTGTTCCCCCCCAGCAGCGAGG |
| | | AGCTGCAGGCCAACAAGGCCACCCT |
| | | GGTGTGCCTGATCAGCGACTTCTACC |
| | | CAGGCGCCGTGACCGTGGCCTGGAA |
| | | GGCCGACAGCAGCCCCGTGAAGGCC |
| | | GGCGTGGAGACCACCACCCCCAGCA |
| | | AGCAGAGCAACAACAAGTACGCCGC |
| | | CAGCAGCTACCTGAGCCTGACCCCC |
| | | GAGCAGTGGAAGAGCCACAGGTCCT |
| | | ACAGCTGCCAGGTGACCCACGAGGG |
| | | CAGCACCGTGGAAAAGACCGTGGCC |
| | | CCAACCGAGTGCAGC |
| mAb2 | | |
| SEQ ID NO: 21 (Kabat) | HCDR1 | VYGMN |
| SEQ ID NO: 22 (Kabat) | HCDR2 | IIWYDGDNQYYADSVKG |
| SEQ ID NO: 23 (Kabat) | HCDR3 | DLRTGPFDY |
| SEQ ID NO: 24 (Chothia) | HCDR1 | GFTFSVY |
| SEQ ID NO: 25 (Chothia) | HCDR2 | WYDGDN |
| SEQ ID NO: 26 (Chothia) | HCDR3 | DLRTGPFDY |
| SEQ ID NO: 27 | VH | QVQLVESGGGVVQPGRSLRLSCAASG |
| | | FTFSVYGMNWVRQAPGKGLEWVAIIW |
| | | YDGDNQYYADSVKGRFTISRDNSKNTL |
| | | YLQMNGLRAEDTAVYYCARDLRTGPF |
| | | DYWGQGTLVTVSS |
| SEQ ID NO: 28 | DNA VH | CAGGTGCAGCTGGTGGAGAGCGGCG |
| | | GCGGCGTGGTGCAGCCCGGCAGGA |
| | | GCCTGAGGCTGAGCTGCGCCGCCAG |
| | | CGGCTTCACCTTCAGCGTGTACGGC |
| | | ATGAACTGGGTGAGGCAGGCCCCCG |
| | | GCAAGGGCCTGGAGTGGGTGGCCAT |
| | | CATCTGGTACGACGGCGACAACCAG |
| | | TACTACGCCGACAGCGTGAAGGGCA |
| | | GGTTCACCATCAGCAGGGACAACAG |
| | | CAAGAACACCCTGTACCTGCAGATGA |
| | | ACGGCCTGAGGGCCGAGGACACCGC |
| | | CGTGTACTACTGCGCCAGGGACCTG |
| | | AGGACCGGCCCCTTCGACTACTGGG |
| | | GCCAGGGCACCCTGGTGACCGTGAG |
| | | CAGC |
| SEQ ID NO: 29 | Heavy Chain | QVQLVESGGGVVQPGRSLRLSCAASG |
| | | FTFSVYGMNWVRQAPGKGLEWVAIIW |
| | | YDGDNQYYADSVKGRFTISRDNSKNTL |
| | | YLQMNGLRAEDTAVYYCARDLRTGPF |
| | | DYWGQGTLVTVSSASTKGPSVFPLAP |
| | | SSKSTSGGTAALGCLVKDYFPEPVTVS |
| | | WNSGALTSGVHTFPAVLQSSGLYSLSS |
| | | VVTVPSSSLGTQTYICNVNHKPSNTKV |
| | | DKRVEPKSCDKTHTCPPCPAPELLGG |
| | | PSVFLFPPKPKDTLMISRTPEVTCVVVD |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPSREEMTKNQVSLTCLVKGF |
| | | YPSDIAVEWESNGQPENNYKTTPPVLD |
| | | SDGSFFLYSKLTVDKSRWQQGNVFSC |
| | | SVMHEALHNHYTQKSLSLSPGK |
| SEQ ID NO: 30 | DNA Heavy Chain | CAGGTGCAGCTGGTGGAGAGCGGCGGCGGCG |
| | | TGGTGCAGCCCGGCAGGAGCCTGAGGCTG |
| | | AGCTGCGCCGCCAGCGGCTTCACCTTCAGCGT |
| | | GTACGGCATGAACTGGGTGAGGCAGGCC |
| | | CCCGGCAAGGGCCTGGAGTGGGTGGCCATCA |
| | | TCTGGTACGACGGCGACAACCAGTACTAC |
| | | GCCGACAGCGTGAAGGGCAGGTTCACCATCA |
| | | GCAGGGACAACAGCAAGAACACCCTGTAC |
| | | CTGCAGATGAACGGCCTGAGGGCCGAGGACA |
| | | CCGCCGTGTACTACTGCGCCAGGGACCTG |
| | | AGGACCGGCCCCTTCGACTACTGGGGCCAGG |
| | | GCACCCTGGTGACCGTGAGCAGCGCCAGC |
| | | ACCAAGGGCCCCAGCGTGTTCCCCCTGGCCCC |
| | | CAGCAGCAAGAGCACCAGCGGCGGCACC |
| | | GCCGCCCTGGGCTGCCTGGTGAAGGACTACTT |
| | | CCCCGAGCCCGTGACCGTGAGCTGGAAC |
| | | AGCGGCGCCCTGACCAGCGGCGTGCACACCTT |
| | | CCCCGCCGTGCTGCAGAGCAGCGGCCTG |
| | | TACAGCCTGAGCAGCGTGGTGACCGTGCCCAG |
| | | CAGCAGCCTGGGCACCCAGACCTACATC |
| | | TGCAACGTGAACCACAAGCCCAGCAACACCAA |
| | | GGTGGACAAGAGGGTGGAGCCCAAGAGC |
| | | TGCGACAAGACCCACACCTGCCCCCCCTGCCCC |
| | | GCCCCCGAGCTGCTGGGCGGCCCCAGC |
| | | GTGTTCCTGTTCCCCCCCAAGCCCAAGGACACC |
| | | CTGATGATCAGCAGGACCCCCGAGGTG |
| | | ACCTGCGTGGTGGTGGACGTGAGCCACGAGG |
| | | ACCCCGAGGTGAAGTTCAACTGGTACGTG |
| | | GACGGCGTGGAGGTGCACAACGCCAAGACCA |
| | | AGCCCAGGGAGGAGCAGTACAACAGCACC |
| | | TACAGGGTGGTGAGCGTGCTGACCGTGCTGCA |
| | | CCAGGACTGGCTGAACGGCAAGGAGTAC |
| | | AAGTGCAAGGTGAGCAACAAGGCCCTGCCCG |
| | | CCCCCATCGAGAAGACCATCAGCAAGGCC |
| | | AAGGGCCAGCCCAGGGAGCCCCAGGTGTACA |
| | | CCCTGCCCCCCAGCAGGGAGGAGATGACC |
| | | AAGAACCAGGTGAGCCTGACCTGCCTGGTGAA |
| | | GGGCTTCTACCCCAGCGACATCGCCGTG |
| | | GAGTGGGAGAGCAACGGCCAGCCCGAGAACA |
| | | ACTACAAGACCACCCCCCCCGTGCTGGAC |
| | | AGCGACGGCAGCTTCTTCCTGTACAGCAAGCT |
| | | GACCGTGGACAAGAGCAGGTGGCAGCAG |
| | | GGCAACGTGTTCAGCTGCAGCGTGATGCACGA |
| | | GGCCCTGCACAACCACTACACCCAGAAG |
| | | AGCCTGAGCCTGAGCCCCGGCAAG |
| SEQ ID NO: 31 (Kabat) | LCDR1 | RASQSIGSSLH |
| SEQ ID NO: 32 (Kabat) | LCDR2 | YASQSFS |
| SEQ ID NO: 33 (Kabat) | LCDR3 | HQSSSLPFT |
| SEQ ID NO: 34 (Chothia) | LCDR1 | SQSIGSS |
| SEQ ID NO: 35 (Chothia) | LCDR2 | YAS |
| SEQ ID NO: 36 (Chothia) | LCDR3 | SSSLPF |
| SEQ ID NO: 37 | VL | EIVLTQSPDFQSVTPKEKVTITCRASQS |
| | | IGSSLHWYQQKPDQSPKLLIKYASQSF |
| | | SGVPSRFSGSGSGTDFTLTINSLEAED |
| | | AAAYYCHQSSSLPFTFGPGTKVDIK |
| SEQ ID NO: 38 | DNA VL | GAGATCGTGCTGACCCAGTCACCCG |
| | | ACTTTCAGTCAGTGACCCCTAAAGAA |
| | | AAAGTGACTATCACCTGTAGGGCCTC |
| | | CCAGTCTATCGGCTCTAGCCTGCACT |
| | | GGTATCAGCAGAAGCCCGATCAGTC |
| | | ACCTAAGCTGCTGATTAAGTACGCCT |
| | | CTCAGTCCTTTAGCGGCGTGCCCTCT |
| | | AGGTTTAGCGGCTCAGGCTCAGGCA |
| | | CCGACTTCACCCTGACTATCAATAGC |
| | | CTGGAAGCCGAGGACGCCGCTGCCT |
| | | ACTACTGTCATCAGTCAAGTAGCCTG |
| | | CCCTTCACCTTCGGCCCTGGCACTAA |
| | | AGTGGATATTAAG |
| SEQ ID NO: 39 | Light Chain | EIVLTQSPDFQSVTPKEKVTITCRASQS |
| | | IGSSLHWYQQKPDQSPKLLIKYASQSF |
| | | SGVPSRFSGSGSGTDFTLTINSLEAED |
| | | AAAYYCHQSSSLPFTFGPGTKVDIKRT |
| | | VAAPSVFIFPPSDEQLKSGTASVVCLLN |
| | | NFYPREAKVQWKVDNALQSGNSQESV |
| | | TEQDSKDSTYSLSSTLTLSKADYEKHK |
| | | VYACEVTHQGLSSPVTKSFNRGEC |
| SEQ ID NO: 40 | DNA Light Chain | GAGATCGTGCTGACCCAGTCACCCG |
| | | ACTTTCAGTCAGTGACCCCTAAAGAA |
| | | AAAGTGACTATCACCTGTAGGGCCTC |
| | | CCAGTCTATCGGCTCTAGCCTGCACT |
| | | GGTATCAGCAGAAGCCCGATCAGTC |
| | | ACCTAAGCTGCTGATTAAGTACGCCT |
| | | CTCAGTCCTTTAGCGGCGTGCCCTCT |
| | | AGGTTTAGCGGCTCAGGCTCAGGCA |
| | | CCGACTTCACCCTGACTATCAATAGC |
| | | CTGGAAGCCGAGGACGCCGCTGCCT |
| | | ACTACTGTCATCAGTCAAGTAGCCTG |
| | | CCCTTCACCTTCGGCCCTGGCACTAA |
| | | AGTGGATATTAAGCGTACGGTGGCC |
| | | GCTCCCAGCGTGTTCATCTTCCCCCC |
| | | CAGCGACGAGCAGCTGAAGAGCGGC |
| | | ACCGCCAGCGTGGTGTGCCTGCTGA |
| | | ACAACTTCTACCCCCGGGAGGCCAA |
| | | GGTGCAGTGGAAGGTGGACAACGCC |
| | | CTGCAGAGCGGCAACAGCCAGGAGA |
| | | GCGTCACCGAGCAGGACAGCAAGGA |
| | | CTCCACCTACAGCCTGAGCAGCACC |
| | | CTGACCCTGAGCAAGGCCGACTACG |
| | | AGAAGCATAAGGTGTACGCCTGCGA |
| | | GGTGACCCACCAGGGCCTGTCCAGC |
| | | CCCGTGACCAAGAGCTTCAACAGGG |
| | | GCGAGTGC |
| Second part from mAb2 | | |
| SEQ ID NO: 41 | HCDR1 | GFTFSVYGMN |
| (Combined) | | |
| SEQ ID NO: 42 | HCDR2 | IIWYDGDNQYYADSVKG |
| (Combined) | | |
| SEQ ID NO: 43 | HCDR3 | DLRTGPFDY |
| (Combined) | | |
| SEQ ID NO: 44 (Kabat) | HCDR1 | VYGMN |
| SEQ ID NO: 45 (Kabat) | HCDR2 | IIWYDGDNQYYADSVKG |
| SEQ ID NO: 46 (Kabat) | HCDR3 | DLRTGPFDY |
| SEQ ID NO: 47 (Chothia) | HCDR1 | GFTFSVY |
| SEQ ID NO: 48 (Chothia) | HCDR2 | WYDGDN |
| SEQ ID NO: 49 (Chothia) | HCDR3 | DLRTGPFDY |
| SEQ ID NO: 50 (IMGT) | HCDR1 | GFTFSVYG |
| SEQ ID NO: 51 (IMGT) | HCDR2 | IWYDGDNQ |
| SEQ ID NO: 52 (IMGT) | HCDR3 | ARDLRTGPFDY |
| SEQ ID NO: 53 | VH | QVQLVESGGGVVQPGRSLRLSCAASG |
| | | FTFSVYGMNWVRQAPGKGLEWVAIIW |
| | | YDGDNQYYADSVKGRFTISRDNSKNTL |
| | | YLQMNGLRAEDTAVYYCARDLRTGPF |
| | | DYWGQGTLVTVSS |
| SEQ ID NO: 54 | DNA VH | CAGGTGCAGCTGGTGGAATCAGGCG |
| | | GCGGAGTGGTGCAGCCTGGTAGATC |
| | | ACTGAGACTGAGCTGCGCTGCTAGT |
| | | GGCTTCACCTTTAGCGTCTACGGAAT |
| | | GAACTGGGTCCGACAGGCCCCTGGG |
| | | AAAGGCCTGGAGTGGGTGGCAATTA |
| | | TCTGGTACGACGGCGATAATCAGTAC |
| | | TACGCCGATAGCGTGAAGGGACGGT |
| | | TCACTATCTCTAGGGATAACTCTAAG |
| | | AACACCCTGTACCTGCAGATGAACGG |
| | | CCTGAGAGCCGAGGACACCGCCGTC |
| | | TACTACTGCGCTAGGGACCTGAGAAC |
| | | CGGCCCCTTCGACTACTGGGGACAG |
| | | GGCACCCTGGTCACCGTGTCTAGC |
| SEQ ID NO: 55 | Heavy Chain | QVQLVESGGGVVQPGRSLRLSCAASG |
| | | FTFSVYGMNWVRQAPGKGLEWVAIIW |
| | | YDGDNQYYADSVKGRFTISRDNSKNTL |
| | | YLQMNGLRAEDTAVYYCARDLRTGPF |
| | | DYWGQGTLVTVSSASTKGPSVFPLAP |
| | | SSKSTSGGTAALGCLVKDYFPEPVTVS |
| | | WNSGALTSGVHTFPAVLQSSGLYSLSS |
| | | VVTVPSSSLGTQTYICNVNHKPSNTKV |
| | | DKRVEPKSCDKTHTCPPCPAPEAAGG |
| | | PSVFLFPPKPKDTLMISRTPEVTCVVVD |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVCTLPPSREEMTKNQVSLSCAVKG |
| | | FYPSDIAVEWESNGQPENNYKTTPPVL |
| | | DSDGSFFLVSKLTVDKSRWQQGNVFS |
| | | CSVMHEALHNHYTQKSLSLSPGK |
| SEQ ID NO: 56 | DNA Heavy Chain | CAGGTGCAGCTGGTGGAATCAGGCG |
| | | GCGGAGTGGTGCAGCCTGGTAGATC |
| | | ACTGAGACTGAGCTGCGCTGCTAGT |
| | | GGCTTCACCTTTAGCGTCTACGGAAT |
| | | GAACTGGGTCCGACAGGCCCCTGGG |
| | | AAAGGCCTGGAGTGGGTGGCAATTA |
| | | TCTGGTACGACGGCGATAATCAGTAC |
| | | TACGCCGATAGCGTGAAGGGACGGT |
| | | TCACTATCTCTAGGGATAACTCTAAG |
| | | AACACCCTGTACCTGCAGATGAACGG |
| | | CCTGAGAGCCGAGGACACCGCCGTC |
| | | TACTACTGCGCTAGGGACCTGAGAAC |
| | | CGGCCCCTTCGACTACTGGGGACAG |
| | | GGCACCCTGGTCACCGTGTCTAGCG |
| | | CCTCTACTAAGGGCCCAAGCGTGTTC |
| | | CCCCTGGCCCCTAGCTCTAAGTCTAC |
| | | TAGCGGAGGCACCGCCGCTCTGGGC |
| | | TGCCTGGTCAAGGACTACTTCCCCGA |
| | | GCCCGTGACCGTCAGCTGGAATAGC |
| | | GGCGCTCTGACTAGCGGAGTGCACA |
| | | CCTTCCCCGCCGTGCTGCAGTCTAG |
| | | CGGCCTGTATAGCCTGTCTAGCGTC |
| | | GTGACCGTGCCTAGCTCTAGCCTGG |
| | | GCACTCAGACCTATATCTGTAACGTG |
| | | AACCACAAGCCCTCTAACACTAAGGT |
| | | GGACAAGCGGGTGGAACCTAAGTCC |
| | | TGCGATAAGACTCACACCTGTCCTCC |
| | | CTGCCCTGCCCCTGAGGCTGCCGGA |
| | | GGACCTAGCGTGTTCCTGTTCCCACC |
| | | TAAGCCTAAAGACACCCTGATGATCT |
| | | CTAGGACCCCCGAAGTGACCTGCGT |
| | | GGTGGTGGACGTCTCACACGAGGAC |
| | | CCTGAAGTGAAGTTTAATTGGTACGT |
| | | GGACGGCGTGGAAGTGCACAACGCT |
| | | AAGACTAAGCCTAGAGAGGAACAGTA |
| | | TAACTCTACCTATAGGGTCGTCAGCG |
| | | TGCTGACAGTGCTGCACCAGGACTG |
| | | GCTGAACGGGAAAGAGTATAAGTGTA |
| | | AAGTGTCTAACAAGGCCCTGCCAGC |
| | | CCCTATCGAAAAGACTATCTCTAAGG |
| | | CTAAGGGGCAGCCTAGAGAACCCCA |
| | | AGTGTGCACTCTGCCCCCTAGTAGAG |
| | | AAGAGATGACTAAGAATCAGGTGTCA |
| | | CTGAGCTGTGCCGTGAAGGGCTTCT |
| | | ACCCTAGCGATATCGCCGTGGAGTG |
| | | GGAGAGCAACGGCCAGCCCGAGAAC |
| | | AACTACAAGACCACCCCCCCAGTGCT |
| | | GGACAGCGACGGCAGCTTCTTCCTG |
| | | GTGAGCAAGCTGACCGTGGACAAGT |
| | | CCAGGTGGCAGCAGGGCAACGTGTT |
| | | CAGCTGCAGCGTGATGCACGAGGCC |
| | | CTGCACAACCACTACACCCAGAAGTC |
| | | CCTGAGCCTGAGCCCCGGCAAG |
| SEQ ID NO: 57 | LCDR1 | RASQSIGSSLH |
| (Combined) | | |
| SEQ ID NO: 58 | LCDR2 | YASQSFS |
| (Combined) | | |
| SEQ ID NO: 59 | LCDR3 | HQSSSLPFT |
| (Combined) | | |
| SEQ ID NO: 60 (Kabat) | LCDR1 | RASQSIGSSLH |
| SEQ ID NO: 61 (Kabat) | LCDR2 | YASQSFS |
| SEQ ID NO: 62 (Kabat) | LCDR3 | HQSSSLPFT |
| SEQ ID NO: 63 (Chothia) | LCDR1 | SQSIGSS |
| SEQ ID NO: 64 (Chothia) | LCDR2 | YAS |
| SEQ ID NO: 65 (Chothia) | LCDR3 | SSSLPF |
| SEQ ID NO: 66 (IMGT) | LCDR1 | QSIGSS |
| SEQ ID NO: 67 (IMGT) | LCDR2 | YASQSFSGVP |
| SEQ ID NO: 68 (IMGT) | LCDR3 | HQSSSLPFT |
| SEQ ID NO: 69 | VL | EIVLTQSPDFQSVTPKEKVTITCRASQS |
| | | IGSSLHWYQQKPDQSPKLLIKYASQSF |
| | | SGVPSRFSGSGSGTDFTLTINSLEAED |
| | | AAAYYCHQSSSLPFTFGPGTKVDIK |
| SEQ ID NO: 70 | DNA VL | GAGATCGTGCTGACCCAGTCACCCG |
| | | ACTTTCAGTCAGTGACCCCTAAAGAA |
| | | AAAGTGACTATCACCTGTAGGGCCTC |
| | | CCAGTCTATCGGCTCTAGCCTGCACT |
| | | GGTATCAGCAGAAGCCCGATCAGTC |
| | | ACCTAAGCTGCTGATTAAGTACGCCT |
| | | CTCAGTCCTTTAGCGGCGTGCCCTCT |
| | | AGGTTTAGCGGCTCAGGCTCAGGCA |
| | | CCGACTTCACCCTGACTATCAATAGC |
| | | CTGGAAGCCGAGGACGCCGCTGCCT |
| | | ACTACTGTCATCAGTCAAGTAGCCTG |
| | | CCCTTCACCTTCGGCCCTGGCACTAA |
| | | AGTGGATATTAAG |
| SEQ ID NO: 71 | Light Chain | EIVLTQSPDFQSVTPKEKVTITCRASQS |
| | | IGSSLHWYQQKPDQSPKLLIKYASQSF |
| | | SGVPSRFSGSGSGTDFTLTINSLEAED |
| | | AAAYYCHQSSSLPFTFGPGTKVDIKRT |
| | | VAAPSVFIFPPSDEQLKSGTASVVCLLN |
| | | NFYPREAKVQWKVDNALQSGNSQESV |
| | | TEQDSKDSTYSLSSTLTLSKADYEKHK |
| | | VYACEVTHQGLSSPVTKSFNRGEC |
| SEQ ID NO: 72 | DNA Light Chain | GAGATCGTGCTGACCCAGTCACCCG |
| | | ACTTTCAGTCAGTGACCCCTAAAGAA |
| | | AAAGTGACTATCACCTGTAGGGCCTC |
| | | CCAGTCTATCGGCTCTAGCCTGCACT |
| | | GGTATCAGCAGAAGCCCGATCAGTC |
| | | ACCTAAGCTGCTGATTAAGTACGCCT |
| | | CTCAGTCCTTTAGCGGCGTGCCCTCT |
| | | AGGTTTAGCGGCTCAGGCTCAGGCA |
| | | CCGACTTCACCCTGACTATCAATAGC |
| | | CTGGAAGCCGAGGACGCCGCTGCCT |
| | | ACTACTGTCATCAGTCAAGTAGCCTG |
| | | CCCTTCACCTTCGGCCCTGGCACTAA |
| | | AGTGGATATTAAGCGTACGGTGGCC |
| | | GCTCCCAGCGTGTTCATCTTCCCCCC |
| | | CAGCGACGAGCAGCTGAAGAGCGGC |
| | | ACCGCCAGCGTGGTGTGCCTGCTGA |
| | | ACAACTTCTACCCCCGGGAGGCCAA |
| | | GGTGCAGTGGAAGGTGGACAACGCC |
| | | CTGCAGAGCGGCAACAGCCAGGAGA |
| | | GCGTCACCGAGCAGGACAGCAAGGA |
| | | CTCCACCTACAGCCTGAGCAGCACC |
| | | CTGACCCTGAGCAAGGCCGACTACG |
| | | AGAAGCATAAGGTGTACGCCTGCGA |
| | | GGTGACCCACCAGGGCCTGTCCAGC |
| | | CCCGTGACCAAGAGCTTCAACAGGG |
| | | GCGAGTGC |
| First part from mAb1 | | |
| SEQ ID NO: 73 | HCDR1 | GGTFKSYAIS |
| (Combined) | | |
| SEQ ID NO: 74 | HCDR2 | NIIPMTGQTYYAQKFQG |
| (Combined) | | |
| SEQ ID NO: 75 | HCDR3 | AAYHPLVFDN |
| (Combined) | | |
| SEQ ID NO: 76 (Kabat) | HCDR1 | SYAIS |
| SEQ ID NO: 77 (Kabat) | HCDR2 | NIIPMTGQTYYAQKFQG |
| SEQ ID NO: 78 (Kabat) | HCDR3 | AAYHPLVFDN |
| SEQ ID NO: 79 (Chothia) | HCDR1 | GGTFKSY |
| SEQ ID NO: 80 (Chothia) | HCDR2 | IPMTGQ |
| SEQ ID NO: 81 (Chothia) | HCDR3 | AAYHPLVFDN |
| SEQ ID NO: 82 (IMGT) | HCDR1 | GGTFKSYA |
| SEQ ID NO: 83 (IMGT) | HCDR2 | IIPMTGQT |
| SEQ ID NO: 84 (IMGT) | HCDR3 | ARAAYHPLVFDN |
| SEQ ID NO: 85 | VH | EVQLVQSGAEVKKPGSSVKVSCKASG |
| | | GTFKSYAISWVRQAPGQGLEWMGNIIP |
| | | MTGQTYYAQKFQGRVTITADESTSTAY |
| | | MELSSLRSEDTAVYYCARAAYHPLVFD |
| | | NWGQGTLVTVSS |
| SEQ ID NO: 86 | DNA VH | GAGGTGCAGCTGGTGCAGTCAGGCG |
| | | CCGAAGTGAAGAAACCCGGCTCTAG |
| | | CGTGAAAGTCAGCTGTAAAGCTAGTG |
| | | GCGGCACCTTCAAGTCCTACGCTATT |
| | | AGCTGGGTCAGACAGGCCCCAGGTC |
| | | AGGGCCTGGAGTGGATGGGCAATAT |
| | | TATCCCTATGACCGGTCAGACCTACT |
| | | ACGCTCAGAAATTTCAGGGTAGAGTG |
| | | ACTATCACCGCCGACGAGTCTACTAG |
| | | CACCGCCTATATGGAACTGTCTAGCC |
| | | TGAGATCAGAGGACACCGCCGTCTA |
| | | CTACTGCGCTAGAGCCGCCTATCACC |
| | | CCCTGGTGTTCGATAACTGGGGTCA |
| | | GGGCACCCTGGTCACCGTGTCTAGC |
| SEQ ID NO: 87 | Heavy Chain | EVQLVQSGAEVKKPGSSVKVSCKASG |
| | | GTFKSYAISWVRQAPGQGLEWMGNIIP |
| | | MTGQTYYAQKFQGRVTITADESTSTAY |
| | | MELSSLRSEDTAVYYCARAAYHPLVFD |
| | | NWGQGTLVTVSSASTKGPSVFPLAPS |
| | | SKSTSGGTAALGCLVKDYFPEPVTVS |
| | | WNSGALTSGVHTFPAVLQSSGLYSLSS |
| | | VVTVPSSSLGTQTYICNVNHKPSNTKV |
| | | DKRVEPKSCDKTHTCPPCPAPEAAGG |
| | | PSVFLFPPKPKDTLMISRTPEVTCVVVD |
| | | VSHEDPEVKFNWYVDGVEVHNAKTKP |
| | | REEQYNSTYRVVSVLTVLHQDWLNGK |
| | | EYKCKVSNKALPAPIEKTISKAKGQPRE |
| | | PQVYTLPPCREEMTKNQVSLWCLVKG |
| | | FYPSDIAVEWESNGQPENNYKTTPPVL |
| | | DSDGSFFLYSKLTVDKSRWQQGNVFS |
| | | CSVMHEALHNHYTQKSLSLSPGK |
| SEQ ID NO: 88 | DNA Heavy Chain | GAGGTGCAGCTGGTGCAGTCAGGCG |
| | | CCGAAGTGAAGAAACCCGGCTCTAG |
| | | CGTGAAAGTCAGCTGTAAAGCTAGTG |
| | | GCGGCACCTTCAAGTCCTACGCTATT |
| | | AGCTGGGTCAGACAGGCCCCAGGTC |
| | | AGGGCCTGGAGTGGATGGGCAATAT |
| | | TATCCCTATGACCGGTCAGACCTACT |
| | | ACGCTCAGAAATTTCAGGGTAGAGTG |
| | | ACTATCACCGCCGACGAGTCTACTAG |
| | | CACCGCCTATATGGAACTGTCTAGCC |
| | | TGAGATCAGAGGACACCGCCGTCTA |
| | | CTACTGCGCTAGAGCCGCCTATCACC |
| | | CCCTGGTGTTCGATAACTGGGGTCA |
| | | GGGCACCCTGGTCACCGTGTCTAGC |
| | | GCTAGCACTAAGGGCCCCTCAGTGTT |
| | | CCCCCTGGCCCCTAGCTCTAAGTCTA |
| | | CTAGCGGCGGCACCGCCGCTCTGGG |
| | | CTGCCTGGTGAAAGACTACTTCCCCG |
| | | AGCCCGTGACCGTGTCATGGAATAG |
| | | CGGCGCTCTGACTAGCGGAGTGCAC |
| | | ACCTTCCCCGCCGTGCTGCAGTCTA |
| | | GCGGCCTGTATAGCCTGTCTAGCGT |
| | | GGTGACCGTGCCTAGCTCTAGCCTG |
| | | GGCACTCAGACCTACATCTGTAACGT |
| | | GAACCACAAGCCCTCTAACACTAAGG |
| | | TGGACAAGCGGGTGGAACCTAAGTC |
| | | CTGCGATAAGACTCACACCTGTCCCC |
| | | CCTGCCCTGCCCCTGAGGCTGCCGG |
| | | AGGACCTAGCGTGTTCCTGTTCCCAC |
| | | CTAAGCCTAAGGACACCCTGATGATC |
| | | TCTAGGACCCCCGAAGTGACCTGCG |
| | | TGGTGGTGGATGTGTCTCACGAGGA |
| | | CCCTGAAGTGAAGTTCAATTGGTACG |
| | | TGGACGGCGTGGAAGTGCACAACGC |
| | | TAAGACTAAGCCTAGAGAGGAACAGT |
| | | ATAACTCCACCTATAGAGTGGTGTCA |
| | | GTGCTGACCGTGCTGCATCAGGACT |
| | | GGCTGAACGGCAAAGAGTATAAGTGT |
| | | AAAGTCTCTAACAAGGCCCTGCCAGC |
| | | CCCTATCGAAAAGACTATCTCTAAGG |
| | | CTAAGGGCCAGCCTAGAGAACCTCA |
| | | GGTGTACACCCTGCCCCCCTGTAGA |
| | | GAAGAGATGACTAAGAATCAGGTGTC |
| | | CCTGTGGTGTCTGGTGAAAGGCTTCT |
| | | ACCCTAGCGATATCGCCGTGGAATG |
| | | GGAGTCTAACGGCCAGCCCGAGAAC |
| | | AACTATAAGACTACCCCCCCTGTGCT |
| | | GGATAGCGACGGCTCATTCTTCCTGT |
| | | ACTCTAAGCTGACCGTGGACAAGTCT |
| | | AGGTGGCAGCAGGGCAATGTGTTTA |
| | | GCTGTAGCGTGATGCACGAGGCCCT |
| | | GCATAATCACTACACTCAGAAGTCAC |
| | | TGAGCCTGAGCCCCGGCAAG |
| SEQ ID NO: 89 | LCDR1 | SGSSSNIGNHYVN |
| (Combined) | | |
| SEQ ID NO: 90 | LCDR2 | RNNHRPS |
| (Combined) | | |
| SEQ ID NO: 91 | LCDR3 | QSWDYSGFSTV |
| (Combined) | | |
| SEQ ID NO: 92 (Kabat) | LCDR1 | SGSSSNIGNHYVN |
| SEQ ID NO: 93 (Kabat) | LCDR2 | RNNHRPS |
| SEQ ID NO: 94 (Kabat) | LCDR3 | QSWDYSGFSTV |
| SEQ ID NO: 95 (Chothia) | LCDR1 | SSSNIGNHY |
| SEQ ID NO: 96 (Chothia) | LCDR2 | RNN |
| SEQ ID NO: 97 (Chothia) | LCDR3 | WDYSGFST |
| SEQ ID NO: 98 (IMGT) | LCDR1 | SSNIGNHY |
| SEQ ID NO: 99 (IMGT) | LCDR2 | RNN |
| SEQ ID NO: 100 (IMGT) | LCDR3 | QSWDYSGFSTV |
| SEQ ID NO: 101 | VL | DIVLTQPPSVSGAPGQRVTISCSGSSS |
| | | NIGNHYVNWYQQLPGTAPKLLIYRNNH |
| | | RPSGVPDRFSGSKSGTSASLAITGLQS |
| | | EDEADYYCQSWDYSGFSTVFGGGTKL |
| | | TVL |
| SEQ ID NO: 102 | DNA VL | GATATCGTCCTGACTCAGCCCCCTAG |
| | | CGTCAGCGGCGCTCCCGGTCAGAGA |
| | | GTGACTATTAGCTGTAGCGGCTCTAG |
| | | CTCTAATATCGGTAATCACTACGTGA |
| | | ACTGGTATCAGCAGCTGCCCGGCAC |
| | | CGCCCCTAAGCTGCTGATCTATAGAA |
| | | ACAATCACCGGCCTAGCGGCGTGCC |
| | | CGATAGGTTTAGCGGATCTAAGTCAG |
| | | GCACTAGCGCTAGTCTGGCTATCACC |
| | | GGACTGCAGTCAGAGGACGAGGCCG |
| | | ACTACTACTGTCAGTCCTGGGACTAT |
| | | AGCGGCTTTAGCACCGTGTTCGGCG |
| | | GAGGCACTAAGCTGACCGTGCTG |
| SEQ ID NO: 103 | Light Chain | DIVLTQPPSVSGAPGQRVTISCSGSSS |
| | | NIGNHYVNWYQQLPGTAPKLLIYRNNH |
| | | RPSGVPDRFSGSKSGTSASLAITGLQS |
| | | EDEADYYCQSWDYSGFSTVFGGGTKL |
| | | TVLGQPKAAPSVTLFPPSSEELQANKA |
| | | TLVCLISDFYPGAVTVAWKADSSPVKA |
| | | GVETTTPSKQSNNKYAASSYLSLTPEQ |
| | | WKSHRSYSCQVTHEGSTVEKTVAPTE |
| | | CS |
| SEQ ID NO: 104 | DNA Light Chain | GATATCGTCCTGACTCAGCCCCCTAG |
| | | CGTCAGCGGCGCTCCCGGTCAGAGA |
| | | GTGACTATTAGCTGTAGCGGCTCTAG |
| | | CTCTAATATCGGTAATCACTACGTGA |
| | | ACTGGTATCAGCAGCTGCCCGGCAC |
| | | CGCCCCTAAGCTGCTGATCTATAGAA |
| | | ACAATCACCGGCCTAGCGGCGTGCC |
| | | CGATAGGTTTAGCGGATCTAAGTCAG |
| | | GCACTAGCGCTAGTCTGGCTATCACC |
| | | GGACTGCAGTCAGAGGACGAGGCCG |
| | | ACTACTACTGTCAGTCCTGGGACTAT |
| | | AGCGGCTTTAGCACCGTGTTCGGCG |
| | | GAGGCACTAAGCTGACCGTGCTGGG |
| | | TCAGCCTAAGGCTGCCCCCAGCGTG |
| | | ACCCTGTTCCCCCCCAGCAGCGAGG |
| | | AGCTGCAGGCCAACAAGGCCACCCT |
| | | GGTGTGCCTGATCAGCGACTTCTACC |
| | | CAGGCGCCGTGACCGTGGCCTGGAA |
| | | GGCCGACAGCAGCCCCGTGAAGGCC |
| | | GGCGTGGAGACCACCACCCCCAGCA |
| | | AGCAGAGCAACAACAAGTACGCCGC |
| | | CAGCAGCTACCTGAGCCTGACCCCC |
| | | GAGCAGTGGAAGAGCCACAGGTCCT |
| | | ACAGCTGCCAGGTGACCCACGAGGG |
| | | CAGCACCGTGGAAAAGACCGTGGCC |
| | | CCAACCGAGTGCAGC |

Throughout the text of this application, should there be a discrepancy between the text of the specification (e.g. Table 15) and the sequence listing, the text of the specification shall prevail.

All patents and publications referenced herein are hereby incorporated by reference in the entirety and for all purposes.

What is claimed is:

1. A method for the treatment or prevention of hidradenitis suppurativa (HS) in a subject in need thereof, comprising administering to said subject a therapeutically effective amount of a bispecific antibody antagonist that specifically binds and inhibits the activity of IL-18 and IL-1β.

2. The method of claim 1, wherein the antibody comprises
a. a first part which is an immunoglobulin with a first variable light chain of (V_{L1}) and a first variable heavy chain (V_{H1}), that binds specifically to a IL1β, and a first constant heavy chain (CH1) with a hetero-dimerization modification, and
b. a second part which is an immunoglobulin with a second variable light chain (V_{L2}) and a second variable heavy chain (V_{H2}), that binds specifically to IL-18 and a second constant heavy chain (CH2) with a hetero-dimerization modification which is complementary to the hetero-dimerization modification of the first constant heavy chain.

3. A method for slowing, arresting, or reducing the development of HS in a subject in need thereof, comprising administering to said subject a therapeutically effective amount of a bispecific antibody antagonist that specifically binds and inhibits the activity of IL-18 and IL-1β.

4. The method of claim 3, wherein the antibody comprises
a. a first part which is an immunoglobulin with a first variable light chain of (V_{L1}) and a first variable heavy chain (V_{H1}), that binds specifically to a IL1β, and a first constant heavy chain (CH1) with a hetero-dimerization modification, and
b. a second part which is an immunoglobulin with a second variable light chain (V_{L2}) and a second variable heavy chain (V_{H2}), that binds specifically to IL-18 and a second constant heavy chain (CH2) with a hetero-dimerization modification which is complementary to the hetero-dimerization modification of the first constant heavy chain.

5. The method according to claim 2 or 4, wherein the first and second constant heavy chain of the bispecific antibody are human IgA, IgD, IgE, IgG, or IgM, preferably IgD, IgE or IgG, such as human IgG1, IgG2, IgG3, or IgG4, preferably IgG1.

6. The method according to claim 2 or 4, wherein the first and second constant heavy chains of the bispecific antibody are IgG1, and wherein
a. the first constant heavy chain has point mutations generating a knob structure and the second constant heavy has point mutations generating a hole structure, or
b. the first constant heavy chain has point mutations generating a hole structure and the second constant heavy has point mutations generating a knob structure, and optionally
C. the first and second constant heavy chains have mutations resulting in a disulfide bridge.

7. The method according to claims 1-6, wherein:
a. the first immunoglobulin V_{H1} domain of the bispecific antibody comprises
i. hypervariable regions CDR1, CDR2 and CDR3, said CDR1 having the amino acid sequence SEQ ID NO:76, said CDR2 having the amino acid sequence SEQ ID NO:77, and said CDR3 having the amino acid sequence SEQ ID NO:78; or
ii. hypervariable regions CDR1, CDR2 and CDR3, said CDR1 having the amino acid sequence SEQ ID NO:79, said CDR2 having the amino acid sequence SEQ ID NO:80, and said CDR3 having the amino acid sequence SEQ ID NO:81; and
b. the first immunoglobulin V_{L1} domain of the bispecific antibody comprises
iii. hypervariable regions CDR1, CDR2 and CDR3, said CDR1 having the amino acid sequence SEQ ID NO:92, said CDR2 having the amino acid sequence SEQ ID NO:93, and said CDR3 having the amino acid sequence SEQ ID NO:94 or
iv. hypervariable regions CDR1, CDR2 and CDR3, said CDR1 having the amino acid sequence SEQ ID NO:95, said CDR2 having the amino acid sequence SEQ ID NO:96, and said CDR3 having the amino acid sequence SEQ ID NO:97; and
c. the second immunoglobulin V_{H2} domain of the bispecific antibody comprises
v. hypervariable regions CDR1, CDR2 and CDR3, said CDR1 having the amino acid sequence SEQ ID NO:44, said CDR2 having the amino acid sequence SEQ ID NO:45, and said CDR3 having the amino acid sequence SEQ ID NO:46; or
vi. hypervariable regions CDR1, CDR2 and CDR3, said CDR1 having the amino acid sequence SEQ ID NO:47, said CDR2 having the amino acid sequence SEQ ID NO:48, and said CDR3 having the amino acid sequence SEQ ID NO:49; and
d. the second immunoglobulin V_{L2} domain of the bispecific antibody comprises
vii. hypervariable regions CDR1, CDR2 and CDR3, said CDR1 having the amino acid sequence SEQ ID NO:60, said CDR2 having the amino acid sequence SEQ ID NO:61, and said CDR3 having the amino acid sequence SEQ ID NO:62 or
viii. hypervariable regions CDR1, CDR2 and CDR3, said CDR1 having the amino acid sequence SEQ ID NO:63, said CDR2 having the amino acid sequence SEQ ID NO:64, and said CDR3 having the amino acid sequence SEQ ID NO:65.

8. The method according to any of the preceding claims, wherein:
a. the first immunoglobulin VH1 domain of the bispecific antibody comprises the amino acid sequence SEQ ID NO: 85,
b. the first immunoglobulin VL1 domain of the bispecific antibody comprises the amino acid sequence SEQ ID NO: 101,
c. the second immunoglobulin VH2 domain of the bispecific antibody comprises the amino acid sequence SEQ ID NO: 53, and
d. the second immunoglobulin VL2 domain of the bispecific antibody comprises the amino acid sequence SEQ ID NO: 69.

9. The method according to any of the preceding claims, wherein:
a. the first immunoglobulin heavy chain of the bispecific antibody comprises the amino acid sequence SEQ ID NO: 87,
b. the first immunoglobulin light chain of the bispecific antibody comprises the amino acid sequence SEQ ID NO: 103,
c. the second immunoglobulin heavy chain of the bispecific antibody comprises the amino acid sequence SEQ ID NO: 55, and
d. the second immunoglobulin light chain of the bispecific antibody comprises the amino acid sequence SEQ ID NO: 71.

10. The method according to any one of the preceding claims, wherein the route of administration is subcutaneous or intravenous, or a combination of subcutaneous or intravenous.

11. The method according to any one of the preceding claims, wherein the dose is about 1.5 mg to about 15 mg active ingredient per kilogram of a human subject.

12. The method according to any one of the preceding claims, wherein the dose is about 5 mg or 10 mg active ingredient per kilogram of a human subject.

13. The method according to any one of the preceding claims, wherein the dose is about 150 mg to about 600 mg active ingredient, such as about 300 mg active ingredient.

14. The method according to any one of the preceding claims, wherein the antibody is administered through a loading dosing and a maintenance dosing.

15. The method according to any one of the preceding claims, wherein the loading dosing is administered via subcutaneous injections of a first dose and the maintenance dosing is administered via subcutaneous injections of a second dose.

16. The method according to claim 15, wherein the first dose is between about 150 mg and about 600 mg active ingredient, such as about 300 mg active ingredient and the second dose is between about 150 mg and about 600 mg active ingredient, such as about 300 mg active ingredient.

17. The method according to claim 15 or 16, wherein the first dose is 150 mg, 300 mg or 600 mg active ingredient and the second dose is 150 mg, 300 mg or 600 mg active ingredient.

18. The method according to any claim 17, wherein the loading dosing comprises at least three subcutaneous biweekly injections on day 1, 15, and 29, and the maintenance dosing consists of monthly (Q4W) subcutaneous injections, beginning on day 57.

19. The method according to any one of the preceding claims, wherein the hidradenitis suppurativa patient is selected according to one of the following criteria:
a. the patient has moderate to severe HS;
b. the patient is an adult;
c. the patient is an adolescent;
d. prior to treatment with the CD40 antagonist, the patient has an HS-PGA score of ≥3;
e. prior to treatment with the CD40 antagonist, the patient has at least 3 inflammatory lesions; or
f. prior to treatment with the CD40 antagonist, the patient does not have extensive scarring (<10 fistulas) as a result of HS.

20. The method according any of claims 1 to 19, wherein by week 16 of treatment the hidradenitis suppurativa patient achieves at least one of the following:
a. a simplified HiSCR;
b. a reduction in HS flares;
c. a NRS30;
d. a reduction of ≤6 as measured by the DLQI; and/or
e. an improvement in DLQI.

21. The method according any of claims 1 to 19, wherein by week 16 of treatment, at least 40% of said patients achieve a simplified HiSCR; or at least 25% of said patients achieve an NRS30 response; or less than 15% of said patients experience an HS flare.

22. An antibody for use according any of claims 1 to 19, wherein the patient has at least one of the following as early as one week after the first dose of the bispecific IL-18 and IL-1β antagonist:
a. a rapid reduction in pain, as measured by VAS or NRS, and
b. a rapid reduction in CRP, as measured using a standard CRP assay.

23. The method according any of claims 1 to 19, wherein the patient achieves a sustained response 3 months after the end of the treatment, as measured by inflammatory lesion count, Hidradenitis Suppurativa Clinical Response (HiSCR), Numerical Rating Scale (NRS), modified Sartorius HS score, Hidradenitis Suppurativa-Physician Global Assessment (HS-PGA), or Dermatology Life Quality Index (DLQI).

24. The method according any of claims 1 to 19, wherein the patient achieves a sustained response 3 months after the end of treatment, as measured by the simplified HiSCR (sHISCR).

25. A pharmaceutical composition comprising a therapeutically effective amount of a bispecific anti-IL-18 and anti-IL-1 β antibody (e.g., bbmAb1) and one or more pharmaceutically acceptable carriers.


SEQUENCE LISTING (104 sequences)

SEQ ID NO: 1 - protein, 5 aa, Artificial Sequence
  source: /note=“Description of Artificial Sequence Synthetic peptide”
SYAIS

SEQ ID NO: 2 - protein, 17 aa, Artificial Sequence
  source: /note=“Description of Artificial Sequence Synthetic peptide”
NIIPMTGQTYYAQKFQG

SEQ ID NO: 3 - protein, 10 aa, Artificial Sequence
  source: /note=“Description of Artificial Sequence Synthetic peptide”
AAYHPLVFDN

SEQ ID NO: 4 - protein, 7 aa, Artificial Sequence
  source: /note=“Description of Artificial Sequence Synthetic peptide”
GGTFKSY

SEQ ID NO: 5 - protein, 6 aa, Artificial Sequence
  source: /note=“Description of Artificial Sequence Synthetic peptide”
IPMTGQ

SEQ ID NO: 6 - protein, 10 aa, Artificial Sequence
  source: /note=“Description of Artificial Sequence Synthetic peptide”
AAYHPLVFDN

SEQ ID NO: 7 - protein, 119 aa, Artificial Sequence
  source: /note=“Description of Artificial Sequence Synthetic polypeptide”
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSS

SEQ ID NO: 8 - DNA, 356 bp, Artificial Sequence
  source: /note=“Description of Artificial Sequence Synthetic polynucleotide”
gaggtgcagctggtgcagagcggcgccgaggtgaagaagcccggcagcagcgtgaaggtgagctgcaaggccagcggcggcaccttcaagagctacgccatcagctgggtgaggcaggcccccggccagggcctggagtggatgggcaacatcatccccatgaccggccagacctactacgcccagaagttccagggcagggtgaccatcaccgccgacgagagcaccagcaccgcctacatggagctgagcagcctgaggagcgaggacaccgccgtgtactactgcgccagggccgcctaccaccccctggtgttcgacaactgggccagggcaccctggtgaccgtgagcagc

SEQ ID NO: 9 - protein, 449 aa, Artificial Sequence
  source: /note=“Description of Artificial Sequence Synthetic polypeptide”
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1347 bp, Artificial Sequence
  source: /note=“Description of Artificial Sequence Synthetic polynucleotide”
gaggtgcagctggtgcagagcggcgccgaggtgaagaagcccggcagcagcgtgaaggtgagctgcaaggccagcggcggcaccttcaagagctacgccatcagctgggtgaggcaggcccccggccagggcctggagtggatgggcaacatcatccccatgaccggccagacctactacgcccagaagttccagggcagggtgaccatcaccgccgacgagagcaccagcaccgcctacatggagctgagcagcctgaggagcgaggacaccgccgtgtactactgcgccagggccgcctaccaccccctggtgttcgacaactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagggtggagcccaagagctgcgacaagacccacacctgccccccctgccccgcccccgaggccgccggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 11 - protein, 13 aa, Artificial Sequence
  source: /note=“Description of Artificial Sequence Synthetic peptide”
SGSSSNIGNHYVN

SEQ ID NO: 12 - protein, 7 aa, Artificial Sequence
  source: /note=“Description of Artificial Sequence Synthetic peptide”
RNNHRPS

SEQ ID NO: 13 - protein, 11 aa, Artificial Sequence
  source: /note=“Description of Artificial Sequence Synthetic peptide”
QSWDYSGFSTV

SEQ ID NO: 14 - protein, 9 aa, Artificial Sequence
  source: /note=“Description of Artificial Sequence Synthetic peptide”
SSSNIGNHY

SEQ ID NO: 15 - protein, 3 aa, Artificial Sequence
  source: /note=“Description of Artificial Sequence Synthetic peptide”
RNN

SEQ ID NO: 16 - protein, 8 aa, Artificial Sequence
  source: /note=“Description of Artificial Sequence Synthetic peptide”
WDYSGFST

SEQ ID NO: 17 - protein, 110 aa, Artificial Sequence
  source: /note=“Description of Artificial Sequence Synthetic polypeptide”
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVL

SEQ ID NO: 18 - DNA, 330 bp, Artificial Sequence
  source: /note=“Description of Artificial Sequence Synthetic polynucleotide”
gatatcgtcctgactcagccccctagcgtcagcggcgctcccggtcagagagtgactattagctgtagcggctctagctctaatatcggtaatcactacgtgaactggtatcagcagctgcccggcaccgcccctaagctgctgatctatagaaacaatcaccggcctagcggcgtgcccgataggtttagcggatctaagtcaggcactagcgctagtctggctatcaccggactgcagtcagaggacgaggccgactactactgtcagtcctgggactatagcggctttagcaccgtgttcggcggaggcactaagctgaccgtgctg

SEQ ID NO: 19 - protein, 216 aa, Artificial Sequence
  source: /note=“Description of Artificial Sequence Synthetic polypeptide”
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - DNA, 648 bp, Artificial Sequence
  source: /note=“Description of Artificial Sequence Synthetic polynucleotide”
gatatcgtcctgactcagccccctagcgtcagcggcgctcccggtcagagagtgactattagctgtagcggctctagctctaatatcggtaatcactacgtgaactggtatcagcagctgcccggcaccgcccctaagctgctgatctatagaaacaatcaccggcctagcggcgtgcccgataggtttagcggatctaagtcaggcactagcgctagtctggctatcaccggactgcagtcagaggacgaggccgactactactgtcagtcctgggactatagcggctttagcaccgtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 21 - protein, 5 aa, Artificial Sequence
  source: /note=“Description of Artificial Sequence Synthetic peptide”
VYGMN

SEQ ID NO: 22 - protein, 17 aa, Artificial Sequence
  source: /note=“Description of Artificial Sequence Synthetic peptide”
IIWYDGDNQYYADSVKG

SEQ ID NO: 23 - protein, 9 aa, Artificial Sequence
  source: /note=“Description of Artificial Sequence Synthetic peptide”
DLRTGPFDY

SEQ ID NO: 24 - protein, 7 aa, Artificial Sequence
  source: /note=“Description of Artificial Sequence Synthetic peptide”
GFTFSVY

SEQ ID NO: 25 - protein, 6 aa, Artificial Sequence
  source: /note=“Description of Artificial Sequence Synthetic peptide”
WYDGDN

SEQ ID NO: 26 - protein, 9 aa, Artificial Sequence
  source: /note=“Description of Artificial Sequence Synthetic peptide”
DLRTGPFDY

SEQ ID NO: 27 - protein, 118 aa, Artificial Sequence [sequence omitted]

SEQ ID NO: 28 - DNA, 354 bp, Artificial Sequence
  source: /note=“Description of Artificial Sequence Synthetic polynucleotide”
caggtgcagctggtggagagcggcggcggcgtggtgcagcccggcaggagcctgaggctgagctgcgccgccagcggcttcaccttcagcgtgtacggcatgaactgggtgaggcaggcccccggcaagggcctggagtgggtggccatcatctggtacgacggcgacaaccagtactacgccgacagcgtgaagggcaggttcaccatcagcagggacaacagcaagaacaccctgtacctgcagatgaacggcctgagggccgaggacaccgccgtgtactactgcgccagggacctgaggaccggccccttcgactactggggccagggcaccctggtgaccgtgagcagc

SEQ ID NO: 29 - protein, 448 aa, Artificial Sequence
  source: /note=“Description of Artificial Sequence Synthetic polypeptide”
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1344 bp, Artificial Sequence
  source: /note=“Description of Artificial Sequence Synthetic polynucleotide”
caggtgcagctggtggagagcggcggcggcgtggtgcagcccggcaggagcctgaggctgagctgcgccgccagcggcttcaccttcagcgtgtacggcatgaactgggtgaggcaggcccccggcaagggcctggagtgggtggccatcatctggtacgacggcgacaaccagtactacgccgacagcgtgaagggcaggttcaccatcagcagggacaacagcaagaacaccctgtacctgcagatgaacggcctgagggccgaggacaccgccgtgtactactgcgccagggacctgaggaccggccccttcgactactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 31 - protein, 11 aa, Artificial Sequence
  source: /note=“Description of Artificial Sequence Synthetic peptide”
RASQSIGSSLH

SEQ ID NO: 32 - protein, 7 aa, Artificial Sequence
  source: /note=“Description of Artificial Sequence Synthetic peptide”
YASQSFS

SEQ ID NO: 33 - protein, 9 aa, Artificial Sequence
  source: /note=“Description of Artificial Sequence Synthetic peptide”
HQSSSLPFT

SEQ ID NO: 34 - protein, 7 aa, Artificial Sequence
  source: /note=“Description of Artificial Sequence Synthetic peptide”
SQSIGSS

SEQ ID NO: 35 - protein, 3 aa, Artificial Sequence
  source: /note=“Description of Artificial Sequence Synthetic peptide”
YAS

SEQ ID NO: 36 - protein, 6 aa, Artificial Sequence
  source: /note=“Description of Artificial Sequence Synthetic peptide”
SSSLPF

SEQ ID NO: 37 - protein, 107 aa, Artificial Sequence [sequence omitted]

SEQ ID NO: 38 - DNA, 321 bp, Artificial Sequence
  source: /note=“Description of Artificial Sequence Synthetic polynucleotide”
gagatcgtgctgacccagtcacccgactttcagtcagtgacccctaaagaaaaagtgactatcacctgtagggcctcccagtctatcggctctagcctgcactggtatcagcagaagcccgatcagtcacctaagctgctgattaagtacgcctctcagtcctttagcggcgtgccctctaggtttagcggctcaggctcaggcaccgacttcaccctgactatcaatagcctggaagccgaggacgccgctgcctactactgtcatcagtcaagtagcctgcccttcaccttcggccctggcactaaagtggatattaag

SEQ ID NO: 39 - protein, 214 aa, Artificial Sequence
  source: /note=“Description of Artificial Sequence Synthetic polypeptide”
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 642 bp, Artificial Sequence
  source: /note=“Description of Artificial Sequence Synthetic polynucleotide”
gagatcgtgctgacccagtcacccgactttcagtcagtgacccctaaagaaaaagtgactatcacctgtagggcctcccagtctatcggctctagcctgcactggtatcagcagaagcccgatcagtcacctaagctgctgattaagtacgcctctcagtcctttagcggcgtgccctctaggtttagcggctcaggctcaggcaccgacttcaccctgactatcaatagcctggaagccgaggacgccgctgcctactactgtcatcagtcaagtagcctgcccttcaccttcggccctggcactaaagtggatattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 41 - protein, 10 aa, Artificial Sequence
  source: /note=“Description of Artificial Sequence Synthetic peptide”
GFTFSVYGMN

SEQ ID NO: 42 - protein, 17 aa, Artificial Sequence
  source: /note=“Description of Artificial Sequence Synthetic peptide”
IIWYDGDNQYYADSVKG

SEQ ID NO: 43 - protein, 9 aa, Artificial Sequence
  source: /note=“Description of Artificial Sequence Synthetic peptide”
DLRTGPFDY

SEQ ID NO: 44 - protein, 5 aa, Artificial Sequence
  source: /note=“Description of Artificial Sequence Synthetic peptide”
VYGMN

SEQ ID NO: 45 - protein, 17 aa, Artificial Sequence
  source: /note=“Description of Artificial Sequence Synthetic peptide”
IIWYDGDNQYYADSVKG

SEQ ID NO: 46 - protein, 9 aa, Artificial Sequence
  source: /note=“Description of Artificial Sequence Synthetic peptide”
DLRTGPFDY

SEQ ID NO: 47 - protein, 7 aa, Artificial Sequence
  source: /note=“Description of Artificial Sequence Synthetic peptide”
GFTFSVY

SEQ ID NO: 48 - protein, 6 aa, Artificial Sequence
  source: /note=“Description of Artificial Sequence Synthetic peptide”
WYDGDN

SEQ ID NO: 49 - protein, 9 aa, Artificial Sequence
  source: /note=“Description of Artificial Sequence Synthetic peptide”
DLRTGPFDY

SEQ ID NO: 50 - protein, 8 aa, Artificial Sequence
  source: /note=“Description of Artificial Sequence Synthetic peptide”
GFTFSVYG

SEQ ID NO: 51 - protein, 8 aa, Artificial Sequence
  source: /note=“Description of Artificial Sequence Synthetic peptide”
IWYDGDNQ

SEQ ID NO: 52 - protein, 11 aa, Artificial Sequence
  source: /note=“Description of Artificial Sequence Synthetic peptide”
ARDLRTGPFDY

SEQ ID NO: 53 - protein, 118 aa, Artificial Sequence [sequence omitted]

SEQ ID NO: 54 - DNA, 354 bp, Artificial Sequence
  source: /note=“Description of Artificial Sequence Synthetic polynucleotide”
caggtgcagctggtggaatcaggcggcggagtggtgcagcctggtagatcactgagactgagctgcgctgctagtggcttcacctttagcgtctacggaatgaactgggtccgacaggcccctgggaaaggcctggagtgggtggcaattatctggtacgacggcgataatcagtactacgccgatagcgtgaagggacggttcactatctctagggataactctaagaacaccctgtacctgcagatgaacggcctgagagccgaggacaccgccgtctactactgcgctagggacctgagaaccggccccttcgactactggggacagggcaccctggtcaccgtgtctagc

SEQ ID NO: 55 - protein, 448 aa, Artificial Sequence
  source: /note=“Description of Artificial Sequence Synthetic polypeptide”
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - DNA, 1344 bp, Artificial Sequence
  source: /note=“Description of Artificial Sequence Synthetic polynucleotide”
caggtgcagctggtggaatcaggcggcggagtggtgcagcctggtagatcactgagactgagctgcgctgctagtggcttcacctttagcgtctacggaatgaactgggtccgacaggcccctgggaaaggcctggagtgggtggcaattatctggtacgacggcgataatcagtactacgccgatagcgtgaagggacggttcactatctctagggataactctaagaacaccctgtacctgcagatgaacggcctgagagccgaggacaccgccgtctactactgcgctagggacctgagaaccggccccttcgactactggggacagggcaccctggtcaccgtgtctagcgcctctactaagggcccaagcgtgttccccctggcccctagctctaagtctactagcggaggcaccgccgctctgggctgcctggtcaaggactacttccccgagcccgtgaccgtcagctggaatagcggcgctctgactagcggagtgcacaccttccccgccgtgctgcagtctagcggcctgtatagcctgtctagcgtcgtgaccgtgcctagctctagcctgggcactcagacctatatctgtaacgtgaaccacaagccctctaacactaaggtggacaagcgggtggaacctaagtcctgcgataagactcacacctgtcctccctgccctgcccctgaggctgccggaggacctagcgtgttcctgttcccacctaagcctaaagacaccctgatgatctctaggacccccgaagtgacctgcgtggtggtggacgtctcacacgaggaccctgaagtgaagtttaattggtacgtggacggcgtggaagtgcacaacgctaagactaagcctagagaggaacagtataactctacctatagggtcgtcagcgtgctgacagtgctgcaccaggactggctgaacgggaaagagtataagtgtaaagtgtctaacaaggccctgccagcccctatcgaaaagactatctctaaggctaaggggcagcctagagaaccccaagtgtgcactctgccccctagtagagaagagatgactaagaatcaggtgtcactgagctgtgccgtgaagggcttctaccctagcgatatcgccgtggagtgggagagcaacggccagcccgagaacaactacaagaccacccccccagtgctggacagcgacggcagcttcttcctggtgagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 57 - protein, 11 aa, Artificial Sequence
  source: /note=“Description of Artificial Sequence Synthetic peptide”
RASQSIGSSLH

SEQ ID NO: 58 - protein, 7 aa, Artificial Sequence
  source: /note=“Description of Artificial Sequence Synthetic peptide”
YASQSFS

SEQ ID NO: 59 - protein, 9 aa, Artificial Sequence
  source: /note=“Description of Artificial Sequence Synthetic peptide”
HQSSSLPFT

SEQ ID NO: 60 - protein, 11 aa, Artificial Sequence
  source: /note=“Description of Artificial Sequence Synthetic peptide”
RASQSIGSSLH

SEQ ID NO: 61 - protein, 7 aa, Artificial Sequence
  source: /note=“Description of Artificial Sequence Synthetic peptide”
YASQSFS

SEQ ID NO: 62 - protein, 9 aa, Artificial Sequence
  source: /note=“Description of Artificial Sequence Synthetic peptide”
HQSSSLPFT

SEQ ID NO: 63 - protein, 7 aa, Artificial Sequence
  source: /note=“Description of Artificial Sequence Synthetic peptide”
SQSIGSS

SEQ ID NO: 64 - protein, 3 aa, Artificial Sequence
  source: /note=“Description of Artificial Sequence Synthetic peptide”
YAS

SEQ ID NO: 65 - protein, 6 aa, Artificial Sequence
  source: /note=“Description of Artificial Sequence Synthetic peptide”
SSSLPF

SEQ ID NO: 66 - protein, 6 aa, Artificial Sequence
  source: /note=“Description of Artificial Sequence Synthetic peptide”
QSIGSS

SEQ ID NO: 67 - protein, 10 aa, Artificial Sequence
  source: /note=“Description of Artificial Sequence Synthetic peptide”
YASQSFSGVP

SEQ ID NO: 68 - protein, 9 aa, Artificial Sequence
  source: /note=“Description of Artificial Sequence Synthetic peptide”
HQSSSLPFT

SEQ ID NO: 69 - protein, 107 aa, Artificial Sequence [sequence omitted]

SEQ ID NO: 70 - DNA, 321 bp, Artificial Sequence; identical to SEQ ID NO: 38
  source: /note=“Description of Artificial Sequence Synthetic polynucleotide”

SEQ ID NO: 71 - protein, 214 aa, Artificial Sequence; identical to SEQ ID NO: 39
  source: /note=“Description of Artificial Sequence Synthetic polypeptide”

SEQ ID NO: 72 - DNA, 642 bp, Artificial Sequence; identical to SEQ ID NO: 40
  source: /note=“Description of Artificial Sequence Synthetic polynucleotide”

SEQ ID NO: 73 - protein, 10 aa, Artificial Sequence
  source: /note=“Description of Artificial Sequence Synthetic peptide”
GGTFKSYAIS

SEQ ID NO: 74 - protein, 17 aa, Artificial Sequence
  source: /note=“Description of Artificial Sequence Synthetic peptide”
NIIPMTGQTYYAQKFQG

SEQ ID NO: 75 - protein, 10 aa, Artificial Sequence
  source: /note=“Description of Artificial Sequence Synthetic peptide”
AAYHPLVFDN

SEQ ID NO: 76 - protein, 5 aa, Artificial Sequence
  source: /note=“Description of Artificial Sequence Synthetic peptide”
SYAIS

SEQ ID NO: 77 - protein, 17 aa, Artificial Sequence
  source: /note=“Description of Artificial Sequence Synthetic peptide”
NIIPMTGQTYYAQKFQG

SEQ ID NO: 78 - protein, 10 aa, Artificial Sequence
  source: /note=“Description of Artificial Sequence Synthetic peptide”
AAYHPLVFDN

SEQ ID NO: 79 - protein, 7 aa, Artificial Sequence
  source: /note=“Description of Artificial Sequence Synthetic peptide”
GGTFKSY

SEQ ID NO: 80 - protein, 6 aa, Artificial Sequence
  source: /note=“Description of Artificial Sequence Synthetic peptide”
IPMTGQ

SEQ ID NO: 81 - protein, 10 aa, Artificial Sequence
  source: /note=“Description of Artificial Sequence Synthetic peptide”
AAYHPLVFDN

SEQ ID NO: 82 - protein, 8 aa, Artificial Sequence
  source: /note=“Description of Artificial Sequence Synthetic peptide”
GGTFKSYA

SEQ ID NO: 83 - protein, 8 aa, Artificial Sequence
  source: /note=“Description of Artificial Sequence Synthetic peptide”
IIPMTGQT

SEQ ID NO: 84 - protein, 12 aa, Artificial Sequence
  source: /note=“Description of Artificial Sequence Synthetic peptide”
ARAAYHPLVFDN

SEQ ID NO: 85 - protein, 119 aa, Artificial Sequence; identical to SEQ ID NO: 7
  source: /note=“Description of Artificial Sequence Synthetic polypeptide”

SEQ ID NO: 86 - DNA, 357 bp, Artificial Sequence
  source: /note=“Description of Artificial Sequence Synthetic polynucleotide”
gaggtgcagctggtgcagtcaggcgccgaagtgaagaaacccggctctagcgtgaaagtcagctgtaaagctagtggcggcaccttcaagtcctacgctattagctgggtcagacaggccccaggtcagggcctggagtggatgggcaatattatccctatgaccggtcagacctactacgctcagaaatttcagggtagagtgactatcaccgccgacgagtctactagcaccgcctatatggaactgtctagcctgagatcagaggacaccgccgtctactactgcgctagagccgcctatcaccccctggtgttcgataactggggtcagggcaccctggtcaccgtgtctagc

SEQ ID NO: 87 - protein, 449 aa, Artificial Sequence
  source: /note=“Description of Artificial Sequence Synthetic polypeptide”
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 1347 bp, Artificial Sequence
  source: /note=“Description of Artificial Sequence Synthetic polynucleotide”
gaggtgcagctggtgcagtcaggcgccgaagtgaagaaacccggctctagcgtgaaagtcagctgtaaagctagtggcggcaccttcaagtcctacgctattagctgggtcagacaggccccaggtcagggcctggagtggatgggcaatattatccctatgaccggtcagacctactacgctcagaaatttcagggtagagtgactatcaccgccgacgagtctactagcaccgcctatatggaactgtctagcctgagatcagaggacaccgccgtctactactgcgctagagccgcctatcaccccctggtgttcgataactggggtcagggcaccctggtcaccgtgtctagcgctagcactaagggcccctcagtgttccccctggcccctagctctaagtctactagcggcggcaccgccgctctgggctgcctggtgaaagactacttccccgagcccgtgaccgtgtcatggaatagcggcgctctgactagcggagtgcacaccttccccgccgtgctgcagtctagcggcctgtatagcctgtctagcgtggtgaccgtgcctagctctagcctgggcactcagacctacatctgtaacgtgaaccacaagccctctaacactaaggtggacaagcgggtggaacctaagtcctgcgataagactcacacctgtcccccctgccctgcccctgaggctgccggaggacctagcgtgttcctgttcccacctaagcctaaggacaccctgatgatctctaggacccccgaagtgacctgcgtggtggtggatgtgtctcacgaggaccctgaagtgaagttcaattggtacgtggacggcgtggaagtgcacaacgctaagactaagcctagagaggaacagtataactccacctatagagtggtgtcagtgctgaccgtgctgcatcaggactggctgaacggcaaagagtataagtgtaaagtctctaacaaggccctgccagcccctatcgaaaagactatctctaaggctaagggccagcctagagaacctcaggtgtacaccctgcccccctgtagagaagagatgactaagaatcaggtgtccctgtggtgtctggtgaaaggcttctaccctagcgatatcgccgtggaatgggagtctaacggccagcccgagaacaactataagactaccccccctgtgctggatagcgacggctcattcttcctgtactctaagctgaccgtggacaagtctaggtggcagcagggcaatgtgtttagctgtagcgtgatgcacgaggccctgcataatcactacactcagaagtcactgagcctgagccccggcaag

SEQ ID NO: 89 - protein, 13 aa, Artificial Sequence
  source: /note=“Description of Artificial Sequence Synthetic peptide”
SGSSSNIGNHYVN

SEQ ID NO: 90 - protein, 7 aa, Artificial Sequence
  source: /note=“Description of Artificial Sequence Synthetic peptide”
RNNHRPS

SEQ ID NO: 91 - protein, 11 aa, Artificial Sequence
  source: /note=“Description of Artificial Sequence Synthetic peptide”
QSWDYSGFSTV

SEQ ID NO: 92 - protein, 13 aa, Artificial Sequence
  source: /note=“Description of Artificial Sequence Synthetic peptide”
SGSSSNIGNHYVN

SEQ ID NO: 93 - protein, 7 aa, Artificial Sequence
  source: /note=“Description of Artificial Sequence Synthetic peptide”
RNNHRPS

SEQ ID NO: 94 - protein, 11 aa, Artificial Sequence
  source: /note=“Description of Artificial Sequence Synthetic peptide”
QSWDYSGFSTV

SEQ ID NO: 95 - protein, 9 aa, Artificial Sequence
  source: /note=“Description of Artificial Sequence Synthetic peptide”
SSSNIGNHY

SEQ ID NO: 96 - protein, 3 aa, Artificial Sequence
  source: /note=“Description of Artificial Sequence Synthetic peptide”
RNN

SEQ ID NO: 97 - protein, 8 aa, Artificial Sequence
  source: /note=“Description of Artificial Sequence Synthetic peptide”
WDYSGFST

SEQ ID NO: 98 - protein, 8 aa, Artificial Sequence
  source: /note=“Description of Artificial Sequence Synthetic peptide”
SSNIGNHY

SEQ ID NO: 99 - protein, 3 aa, Artificial Sequence
  source: /note=“Description of Artificial Sequence Synthetic peptide”
RNN

SEQ ID NO: 100 - protein, 11 aa, Artificial Sequence
  source: /note=“Description of Artificial Sequence Synthetic peptide”
QSWDYSGFSTV

SEQ ID NO: 101 - protein, 110 aa, Artificial Sequence; identical to SEQ ID NO: 17
  source: /note=“Description of Artificial Sequence Synthetic polypeptide”

SEQ ID NO: 102 - DNA, 330 bp, Artificial Sequence; identical to SEQ ID NO: 18
  source: /note=“Description of Artificial Sequence Synthetic polynucleotide”

SEQ ID NO: 103 - protein, 216 aa, Artificial Sequence; identical to SEQ ID NO: 19
  source: /note=“Description of Artificial Sequence Synthetic polypeptide”

SEQ ID NO: 104 - DNA, 648 bp, Artificial Sequence; identical to SEQ ID NO: 20
  source: /note=“Description of Artificial Sequence Synthetic polynucleotide”